SEQUENCE LISTING (57 sequences)

SEQ ID NO: 1 - protein, 738 aa, Artificial Sequence
  Description of Artificial Sequence Synthetic polypeptide
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, Artificial Sequence
  Description of Artificial Sequence Synthetic polypeptide
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 3 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 4 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 5 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 6 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFKGEQMFDVKEAIDTKNHQLDSKLFEYFKEKAFPYLSTKHLGVFPDHVIDMFINGYRLSLTNHGPTPVKRGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 7 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 8 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIKRYLREHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELDEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 9 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFNGEQMFDVKEAIRTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELEEGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 10 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIERYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEKNIGAQVLGLFTLSTGQKSWNQTN

SEQ ID NO: 11 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFRGEQMFDVKEAIRTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKSELENGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 12 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFRGEQMFDVKEAIRTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKKELEEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 13 - protein, 310 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIERYLEEHPEKQKINFRGEQMFDVKEAIDTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTN

SEQ ID NO: 14 - protein, 306 aa, Artificial Sequence
  Description of Artificial Sequence Synthetic polypeptide
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSW

SEQ ID NO: 15 - protein, 290 aa, Artificial Sequence
  Description of Artificial Sequence Synthetic polypeptide
SVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 16 - protein, 290 aa, Artificial Sequence
  Description of Artificial Sequence Synthetic polypeptide
SVWTKGVTPPANFTQGEDVFHAPYVANQGWYDITKTFNGKDDLLCGAATAGNMLHWWFDQNKDQIKRYLEEHPEKQKINFKGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 17 - protein, 290 aa, Artificial Sequence
  Description of Artificial Sequence Synthetic polypeptide
SVWTKGVTPPANFTQGEDVFHAPYVANQGWYDITKTFNGKDDLLCGAATAGNMLHWWFDQNKDQIERYLEEHPEKQKINFKGEQMFDVKKAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTN

SEQ ID NO: 18 - protein, 319 aa, Artificial Sequence
  Description of Artificial Sequence Synthetic polypeptide
DDYQRNATEAYAKEVPHQITSVWTKGVTPPANFTQGEDVFHAPYVANQGWYDITKTFNGKDDLLCGAATAGNMLHWWFDQNKDQIERYLEEHPEKQKINFKGEQMFDVKKAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTNGGGHHHHHH

SEQ ID NO: 19 - protein, 586 aa, Streptococcus agalactiae
NQNNIQETNLVEKNSEDKFIQELNRYKTEIPNFKGFNVWILGDKGYYKNLINLEEIKNIQATLKKERNEEYVFVKLNGKIAHDTTVFLMNKKHKLLKNIEEFKTITQKRLTERGKFPYDTVHSTFEIKDENFIMERLKSSGLSMGKPVDYMGVNGIPIYTKTLSIDNKFAFENNSKDSSYSSNINISEDKIKENDQKILDLIVKSGANNQNLTDEEKVIAFTKYIGEITNYDNEAYRARNVDTEYYRASDLFSVTERKLAMCVGYSVTAARAFNIMGIPSYVVSGKSPQGISHAAVRAYYNRSWHIIDITASTYWKNGNYKTTYSDFIKEYCIDGYDVYDPAKTNNRFKVKYMESNEAFENWIHNNGSKSMLFINESAALKDKKPKDDFVPVTEKEKNELIDKYKKLLSQIPENTQNPGEKNIRDYLKNEYEEILKKDNLFEHEHAEFKESLNLNESFYLQLKKEEKKPSDNLKKEEKPRENSVKERETPAENNDFVSVTEKNNLIDKYKELLSKIPENTQNPGEKNIRNYLEKEYEELLQKDKLFKHEYTEFTKSLNLNETFYSQLKEGEMKLSENPEKGETNTN

SEQ ID NO: 20 - protein, 497 aa, Streptococcus pseudoporcinus
RENENVRQLQSENKQMKAVNLQEFSEKLKGEIAENQQFHIFKLGLNNYYIGGVRINELSDLAKNHDFIMIDNRATHNKYGVPHIIIMNKDDVIVHNQEDYNKEMAELTFAGDKPIQSDSYLPQKKRIHALFEIGLDSNRRQLLNAAGLKTPENSVIELDTFKIYSHGLAVDNKYYDEYSHFNNNTNVNITKQRFTENDNLIHNLITTSTAKDQPTDRDKVKTFVMYVANHTIYDWNAANNAVSNISDVNYYLGSDLFSITERKKAMCVGFSTTAARAFNMLGIPAYVVEGKNAQGVDHATARVYYNGKWHTIDGTGFINGNRTRSTLYTESHFRSVGEDSYQLVGLNEDIPFDRNYMKIDKVYEEWAPKQKTADLLLVNKDKSLVGLDRVAYVEPVYVDKNRQDALTQIYKKLKETMESSSKKNPSSGGFSSLLGSASSDIAKLEGSSQLTQEEYDKIHRSMTSILTFFAQLDKDAAEAFEKGNDYKNYLATTKHAQ

SEQ ID NO: 21 - protein, 349 aa, Streptococcus equi
MKTIAYPNKPHSLSAGLLTAIAIFSLASSNITYADDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 22 - protein, 315 aa, Streptococcus equi
DDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 23 - protein, 313 aa, Artificial Sequence
  Description of Artificial Sequence Synthetic polypeptide
DDYQRNATEAYAKEVPHQITSVWTKGVTPLTPEQFRYNNEDVFHAPYVANQGWYDITKAFDGKDNLLCGAATAGNMLHWWFDQNKDQIKRYLEEHPEKQKINFNGDNMFDVKKAIDTKNHQLDSKLFNYFKEKAFPGLSARRIGVFPDHVIDMFINGYRLSLTNHGPTPVKEGSKDPRGGIFDAVFTRGNQSKLLTSRHDFKNKNLNDISTIIKQELTKGKALGLSHTYANVSINHVINLWGADFNAEGNLEAIYVTDSDSNASIGMKKYFVGVNAHGHVAISAKKIEGENIGAQVLGLFTLSTGQDSWQKLS

SEQ ID NO: 24 - protein, 315 aa, Artificial Sequence
  Description of Artificial Sequence Synthetic polypeptide
DDYQRNATEAYAKEVPHQITSVWTKGVTPLTPEQFRYNNEDVIHAPYLANQGWYDITKAFDGKDNLLCGAATAGNMLHWWFDQNKTEIEAYLSKHPEKQKIIFRNQELFDLKEAIRTKDSQTNSQLFEYFRDKAFPYLSARQLGVMPDLVLDMFINGYYLNVFKTQSTDVKRPYQDKDKRGGIFDAVFTRGNQTTLLTARHDLKNKGLNDISTIIKEELTKGRALALSHTYANVSISHVINLWGADFNAEGNLEAIYVTDSDANASIGMKKYFVGINKHGHVAISAKKIEGENIGAQVLGLFTLSSGKDIWQKLN

SEQ ID NO: 25 - protein, 315 aa, Artificial Sequence
  Description of Artificial Sequence Synthetic polypeptide
DDYQRNATEAYAKEVPHQITSVWTKGVTPLTPEQFRYNNEDVIHAPYLAHQGWYDITKTFNGKDNLLCGAATAGNMLHWWFDQNKTEIEAYLSKHPEKQKIIFNNEELFDLKAAIDTKDSQTNSQLFNYFKEKAFPNLSTRQLGVMPDLVLDMFINGYYLNVFKTQSTDVNRPYQDKDKRGGIFDAVFTRGNQTTLLTARHDFKEKGLKDISTIIKQELTEGRALALSHTYANVSISHVINLWGADFDAEGNLKAIYVTDSDANASIGMKKYFVGINAHGKVAISAKKIEGENIGAQVLGLFTLSSGKDIWQQLS

SEQ ID NO: 26 - protein, 315 aa, Artificial Sequence
  Description of Artificial Sequence Synthetic polypeptide
DSFSANQEIRYSEVTPYHVTSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 27 - protein, 295 aa, Artificial Sequence
  Description of Artificial Sequence Synthetic polypeptide
SVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 28 - protein, 314 aa, Artificial Sequence
  Description of Artificial Sequence Synthetic polypeptide
DDYQRNATEAYAKEVPHQITSVWTKGVTPLTPEQFTQGEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 29 - protein, 312 aa, Artificial Sequence
  Description of Artificial Sequence Synthetic polypeptide
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 30 - protein, 313 aa, Artificial Sequence
  Description of Artificial Sequence Synthetic polypeptide
DDYQRNATEAYAKEVPHQITSVWTKGVTPPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 31 - protein, 312 aa, Artificial Sequence
  Description of Artificial Sequence Synthetic polypeptide
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIINNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 32 - protein, 312 aa, Artificial Sequence
  Description of Artificial Sequence Synthetic polypeptide
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 33 - protein, 312 aa, Artificial Sequence
  Description of Artificial Sequence Synthetic polypeptide
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIIRNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 34 - protein, 312 aa, Artificial Sequence
  Description of Artificial Sequence Synthetic polypeptide
DDYQRNATEAYAKEVPHQITSVWTKGVTPPEQFTQGEDVIHAPYLANQGWYDITKAFDGKDNLLCGAATAGNMLHWWFDQNKTEIEAYLSKHPEKQKIIFRNQELFDLKEAIRTKDSQTNSQLFEYFRDKAFPYLSARQLGVMPDLVLDMFINGYYLNVFKTQSTDVKRPYQDKDKRGGIFDAVFTRGNQTTLLTARHDLKNKGLNDISTIIKEELTKGRALALSHTYANVSISHVINLWGADFNAEGNLEAIYVTDSDANASIGMKKYFVGINKHGHVAISAKKIEGENIGAQVLGLFTLSSGKDIWQKLN

SEQ ID NO: 35 - protein, 292 aa, Artificial Sequence
  Description of Artificial Sequence Synthetic polypeptide
SVWTKGVTPPEQFTQGEDVIHAPYLAHQGWYDITKAFDGKDNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGNQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 36 - protein, 312 aa, Artificial Sequence
  Description of Artificial Sequence Synthetic polypeptide
DDYQRNATEAYAKEVPHQITSVWTKGVTPPEQFTQGEDVIHAPYLAHQGWYDITKAFDGADNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGNQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 37 - protein, 292 aa, Artificial Sequence
  Description of Artificial Sequence Synthetic polypeptide
SVWTKGVTPPEQFTQGEDVIHAPYLAHQGWYDITKAFDGADNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGNQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 38 - protein, 312 aa, Artificial Sequence
  Description of Artificial Sequence Synthetic polypeptide
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGNQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 39 - protein, 312 aa, Artificial Sequence
  Description of Artificial Sequence Synthetic polypeptide
DDYQRNATEAYAKEVPHQITSVWTKGVTPLTPEQFTQGEDVFHAPYVANQGWYDITKAFDGKDNLLCGAATAGNMLHWWFDQNKDQIKRYLEEHPEKQKINFNGENMFDVKKAIDTKNHQLDSKLFNYFKEKAFPYLSAKHLGVFPDHVIDMFINGYRLSLTNHGPTPVKEGSKDPRGGIFDAVFTRGNQSKLLTSRHDFKNKNLNDISTIIKQELTKGKALGLSHTYANVRINHVINLWGADFNAEGNLEAIYVTDSDSNASIGMKKYFVGVNAHGHVAISAKKIEGENIGAQVLGLFTLSTGQDSWQKLS

SEQ ID NO: 40 - protein, 312 aa, Artificial Sequence
  Description of Artificial Sequence Synthetic polypeptide
DDYQRNATEAYAKEVPHQITSVWTKGVTPLTPEQFTQGEDVFHAPYVANQGWYDITKAFDGKDNLLCGAATAGNMLHWWFDQNKDQIKRYLEEHPEKQKINFRGENMFDVKEAIRTKNHQLDSKLFEYFKEKAFPYLSAKHLGVFPDHVIDMFINGYRLSLTNHGPTPVKKGSKDPRGGIFDAVFTRGNQSKLLTSRHDFKNKNLNDISTIIKSELTNGKALGLSHTYANVRINHVINLWGADFNAEGNLEAIYVTDSDSNASIGMKKYFVGVNKHGHVAISAKKIEGENIGAQVLGLFTLSTGQDSWQKLN

SEQ ID NO: 41 - protein, 312 aa, Artificial Sequence
  Description of Artificial Sequence Synthetic polypeptide
DDYQRNATEAYAKEVPHQITSVWTKGVTPLTPEQFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKQELTEGKALGLSHTYANVRINHVINLWGADFDAEGNLKAIYVTDSDSNASIGMKKYFVGVNAAGKVAISAKKIEGENIGAQVLGLFTLSTGQDSWNQTS

SEQ ID NO: 42 - protein, 312 aa, Artificial Sequence
  Description of Artificial Sequence Synthetic polypeptide
DDYQRNATEAYAKEVPHQITSVWTKGVTPLTPEQFTQGEDVFHAPYVANQGWYDITKTFNGKDDLLCGAATAGNMLHWWFDQNKDQIKRYLEEHPEKQKINFRGEQMFDVKEAIRTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKEELTKGKALGLSHTYANVRINHVINLWGADFDAEGNLKAIYVTDSDSNASIGMKKYFVGVNKAGKVAISAKKIEGENIGAQVLGLFTLSTGQDSWNQTN

SEQ ID NO: 43 - protein, 310 aa, Artificial Sequence
  Description of Artificial Sequence Synthetic polypeptide
DDYQRNATEAYAKEVPHQITSVWTKGVTPPEQFTQGEDVIHAPYVANQGWYDITKAFDGKDNLLCGAATAGNMLHWWFDQNKDQIKRYLEEHPEKQKINFRGEQMFDVKKAIDTKNHQLDSKLFNYFKEKAFPGLSARRIGVFPDHVIDMFINGYRLSLTNHGPTPVKEGSKDPRGGIFDAVFTRGNQSKLLTSRHDFKNKNLNDISTIIKQELTKGKALGLSHTYANVSINHVINLWGADFNAEGNLEAIYVTDSDSNASIGMKKYFVGVNAHGHVAISAKKIEGENIGAQVLGLFTLSTGQDSWQKLS

SEQ ID NO: 44 - protein, 843 aa, Streptococcus pyogenes
MDKHLLVKRTLGCVCAATLMGAALATHHDSLNTVKAEEKTVQTGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKD

SEQ ID NO: 45 - protein, 959 aa, Streptococcus pyogenes
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 46 - protein, 995 aa, Streptococcus pyogenes
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 47 - protein, 995 aa, Streptococcus pyogenes; identical to SEQ ID NO: 46

SEQ ID NO: 48 - protein, 311 aa, Artificial Sequence
  Description of Artificial Sequence Synthetic polypeptide
MDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 49 - protein, 216 aa, Artificial Sequence
  Description of Artificial Sequence Synthetic polypeptide
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 50 - protein, 445 aa, Artificial Sequence
  Description of Artificial Sequence Synthetic polypeptide
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 14 aa, Artificial Sequence
  Description of Artificial Sequence Synthetic peptide
TGTGSDVGSYNLVS

SEQ ID NO: 52 - protein, 7 aa, Artificial Sequence
  Description of Artificial Sequence Synthetic peptide
GDSERPS

SEQ ID NO: 53 - protein, 10 aa, Artificial Sequence
  Description of Artificial Sequence Synthetic peptide
SSYAGSGIYV

SEQ ID NO: 54 - protein, 5 aa, Artificial Sequence
  Description of Artificial Sequence Synthetic peptide
TYAMG

SEQ ID NO: 55 - protein, 14 aa, Artificial Sequence
  Description of Artificial Sequence Synthetic peptide
SIGASGSQTRYADS

SEQ ID NO: 56 - protein, 7 aa, Artificial Sequence
  Description of Artificial Sequence Synthetic peptide
LAIGDSY

SEQ ID NO: 57 - protein, 7 aa, Artificial Sequence
  Description of Artificial Sequence Synthetic peptide
  MOD_RES (6)..(6): A hydrophobic amino acid
GHFGGXY

(19) United States
(12) Patent Application Publication
(10) Pub. No.: US 2023/0241247 A1
(43) Pub. Date: Aug. 3, 2023

(54) COMPOSITIONS AND METHODS FOR INCREASING OR ENHANCING TRANSDUCTION OF GENE THERAPY VECTORS AND FOR REMOVING OR REDUCING IMMUNOGLOBULINS

(71) Applicant: SPARK THERAPEUTICS, INC., Philadelphia, PA (US)

(72) Inventor: Sean ARMOUR, Holland, PA (US)

(73) Assignee: SPARK THERAPEUTICS, INC., Philadelphia, PA (US)

(21) Appl. No.: 17/759,183

(22) PCT Filed: Jan. 22, 2021
(86) PCT No.: PCT/US2021/014770

§ 371 Date: Jul. 20, 2022

Related U.S. Application Data
(60) Provisional application No. 62/964,565, filed on Jan. 22, 2020.

(51) Int. Cl. A61K 48/00; C07K 16/28; A61K 38/48; A61K 38/47; A61K 38/46; A61K 31/7088; C12N 15/86; C12N 15/11; C12N 9/22; G01N 33/569

(52) U.S. Cl. CPC A61K 48/0041; C07K 16/283; A61K 38/48; A61K 38/47; A61K 48/0083; A61K 38/465; A61K 31/7088; C12N 15/86; C12N 15/11; C12N 9/22; G01N 33/56983; C12N 2310/20; C12N 2800/80; C12N 2750/14143; C12N 2750/14145; C12N 2750/14171; G01N 2469/20

(57) ABSTRACT

Disclosed herein are methods for treating patients that may develop or already have pre-existing gene therapy neutralizing antibodies by administering an agent that blocks, inhibits or reduces the interaction between immunoglobulin G (IgG) and the neonatal Fc receptor (FcRn), such as an anti-FcRn antibody, to reduce IgG recycling and enhance IgG clearance in vivo. Also disclosed are methods for utilizing agents that reduce interaction of IgG with FcRn for gene therapy treatment of a disease in a patient in need thereof.

### RELATED APPLICATIONS

This patent application is the National Phase of International Application No. PCT/US2021/014770, filed Jan. 22, 2021, which designated the U.S. and that International Application was published under PCT Article 21(2) in English, which claims the benefit of priority to U.S. Provisional Patent Application No. 62/964,565, filed Jan. 22, 2020. The entire content of the foregoing application is incorporated herein by reference, including all text, tables, sequence listing and drawings.

### SEQUENCE LISTING

The instant application contains a Sequence Listing which has been submitted electronically in ASCII text format and is hereby incorporated by reference in its entirety. Said ASCII text copy, created on Dec. 29, 2022, is named “023637-0569872_SEQUENCE_LISTING_ST25.txt and is 174,361 bytes in size.

### INTRODUCTION

Adeno-associated virus (AAV) and other viral vectors as well as lipid-, polymer-, and protein-based nanoparticle gene therapy approaches can be targeted by the adaptive immune system, leading to blunted efficacy and the possibility of a patient becoming completely refractory to therapeutic intervention. The adaptive-immune system relies on development of antigen-specific immunoglobulin (e.g., IgG) antibodies which lead to the inhibition or clearance of the target molecule.

The neonatal Fc receptor (FcRn) plays a central role in the recycling of immunoglobulins of the G type (IgGs) away from the lysosomal pathway, back to the plasma membrane and outside the cell, thus extending IgG serum half-life (see, Sockolosky et al. 2015, Adv. Drug Deliv. Rev., 91:109-124). Agents that reduce the interaction of IgG with FcRn decrease IgG recycling, enhance IgG degradation/catabolism, and decrease IgG serum half-life. In a human clinical trial, multiple weekly doses of an anti-FcRn antibody resulted in an average serum IgG reduction of ˜85%, with sustained serum IgG reduction of ≥75% for up to 24 days (Ling et al., 2019, Clin. Pharmacol. Ther., 105:1031-1039).

IdeS is a naturally occurring cysteine protease, specifically an endopeptidase, expressed by the pathogenic bacteria Streptococcus pyogenes that exhibits specificity for its target sequence found in human IgG, in addition to several other species. IdeS is capable of cleaving IgG below the hinge region, leading to the generation of F(ab′)2 and Fc/2 fragments. IdeS is capable of cleaving IgG in human plasma, and can reduce total IgG levels in humans between 4 hours to 7 days post administration.

EndoS is a naturally occurring glycosidase, specifically an endoglycosidase, from S. pyogenes, that specifically hydrolyzes glycans from human IgG and alters antibody effector functions, including Fc receptor binding.

Neutralizing antibodies to the AAV capsid are a major hurdle to gene therapy vectors, leaving certain patients without access to potentially life-saving therapies. Described herein are, inter alia, methods for treating patients that may develop or already have pre-existing neutralizing antibodies to gene therapy vectors by administering an agent that reduces the interaction of IgG with FcRn, thereby reducing IgG recycling, enhancing IgG clearance, degradation and catabolism, and decreasing circulating IgG or IgG half-life in vivo. Also described herein are, inter alia, methods for treating patients that may develop or already have pre-existing antibodies that bind to a heterologous polynucleotide or a protein or peptide encoded by the heterologous polynucleotide encapsidated by a gene therapy vector by administering an agent that reduces the interaction of IgG with FcRn, thereby reducing IgG recycling, enhancing IgG clearance, degradation and catabolism, and decreasing circulating IgG or IgG half-life in vivo.

### SUMMARY

Disclosed herein are methods for utilizing an agent that reduces the interaction of immunoglobulin G (IgG) with the neonatal Fc receptor (FcRn) and FcRn-mediated IgG recycling to reduce circulating levels or titer of antibody (e.g., IgG in a subject (e.g., human patient), to improve or enhance gene therapy in the subject. Methods according to the invention may be used, inter alia, to treat patients with pre-existing neutralizing antibodies to gene therapy vectors and to re-dose patients previously treated with a gene therapy vector.

In certain embodiments, a method of enhancing the efficacy of gene therapy treatment in a subject, comprising (a) administering to a subject an agent that reduces the interaction of IgG with the FcRn, and (b) administering to the subject a recombinant viral vector comprising a therapeutic heterologous polynucleotide.

In certain embodiments, the subject is in need of treatment for a disease caused by a loss of function or activity of a protein, and the therapeutic heterologous polynucleotide encodes a polypeptide or peptide that provides or supplements a function or activity of the protein, or the subject is in need of treatment for a disease caused by a gain of function, activity or expression of a protein, and the heterologous polynucleotide is transcribed into a nucleic acid that inhibits, decreases or reduces expression of the gain of function, activity or expression of the protein.

In certain embodiments, FcRn-mediated IgG recycling is reduced in the subject.

In certain embodiments, IgG clearance is enhanced in the subject.

In certain embodiments, the agent that reduces the interaction of IgG with the FcRn is selected from the group consisting of an anti-FcRn antibody, an FcRn binding affibody, an antibody that enhances IgG degradation (ABDEG), an FcRn binding peptide (FcBP), and a small molecule FcRn antagonist.

In certain embodiments, in methods of treating a subject, step (a) is performed before step (b) is performed.

In certain embodiments, in methods of treating a subject, step (b) is performed before step (a) is performed.

In certain embodiments, in methods of treating a subject, step (a) and step (b) are performed at about the same time.

In certain embodiments, in methods of treating a subject, step (a) is performed two or more times before or after step (b) is performed.

In certain embodiments, in methods of treating a subject, step (b) is performed within about 90 days before or after step (a) is performed. In certain embodiments, step (b) is performed within about 60 days before or after step (a) is performed. In certain embodiments, step (b) is performed within about 45 days before or after step (a) is performed. In certain embodiments, step (b) is performed within about 30 days before or after step (a) is performed. In certain embodiments, step (b) is performed within about 21 days before or after step (a) is performed. In certain embodiments, step (b) is performed within about 14 days before or after step (a) is performed. In certain embodiments, step (b) is performed within about 7 days before or after step (a) is performed. In certain embodiments, step (b) is performed within about 72 hours before or after step (a) is performed. In certain embodiments, step (b) is performed within about 48 hours before or after step (a) is performed. In certain embodiments, step (b) is performed within about 24 hours before or after step (a) is performed. In certain embodiments, step (b) is performed within about 12 hours before or after step (a) is performed. In certain embodiments, step (b) is performed within about 6 hours before or after step (a) is performed.

In certain embodiments, a method further comprising administering to the subject an amount of a protease or glycosidase effective to degrade or digest and/or inhibit or reduce effector function of antibodies that bind to the recombinant viral vector and/or the polypeptide or peptide encoded by the heterologous polynucleotide and/or the heterologous polynucleotide.

In certain embodiments, the protease or glycosidase is administered before, after or at about the same time as step (a). In certain embodiments, the protease or glycosidase is administered before, after or at about the same time as step (b). In certain embodiments, the protease or glycosidase is administered two or more times before, after or at about the same time as step (a). In certain embodiments, the protease or glycosidase is administered two or more times before, after or at about the same time as step (b). In certain embodiments, the protease or glycosidase is administered within about 90 days before or after step (a) or step (b). In certain embodiments, the protease or glycosidase is administered within about 60 days before or after step (a) or step (b). In certain embodiments, the protease or glycosidase is administered within about 45 days before or after step (a) or step (b). In certain embodiments, the protease or glycosidase is administered within about 30 days before or after step (a) or step (b). In certain embodiments, the protease or glycosidase is administered within about 21 days before or after step (a) or step (b). In certain embodiments, the protease or glycosidase is administered within about 14 days before or after step (a) or step (b). In certain embodiments, the protease or glycosidase is administered within about 7 days before or after step (a) or step (b). In certain embodiments, the protease or glycosidase is administered within about 72 hours before or after step (a) or step (b). In certain embodiments, the protease or glycosidase is administered within about 48 hours before or after step (a) or step (b). In certain embodiments, the protease or glycosidase is administered within about 24 hours before or after step (a) or step (b). In certain embodiments, the protease or glycosidase is administered within about 12 hours before or after step (a) or step (b). In certain embodiments, the protease or glycosidase is administered within about 6 hours before or after step (a) or step (b).

In certain embodiments, the protease comprises a cysteine protease or a thiol protease.

In certain embodiments, the protease comprises a protease from Streptococcus pyogenes, Streptococcus equi or Mycoplasma canis.

In certain embodiments, the protease comprises IdeS or a modified variant thereof set forth in any of SEQ ID NOs:3-18, 23 or 48.

In certain embodiments, the glycosidase comprises an endoglycosidase.

In certain embodiments, the endoglycosidase comprises a sequence set forth in any of SEQ ID NOs: 44-47.

In certain embodiments, the protease or glycosidase degrades or digests and/or inhibits or reduces effector function of human antibodies.

In certain embodiments, the viral vector comprises a lentiviral vector, an adenoviral vector or an adeno-associated virus (AAV) vector.

In certain embodiments, the lentiviral vector comprises envelope proteins to which the antibodies or IgG bind.

In certain embodiments, the AAV vector comprises capsid proteins to which the antibodies or IgG bind.

In certain embodiments, the AAV vector comprises VP1, VP2 and/or VP3 capsid proteins to which the antibodies or IgG bind.

In certain embodiments, the AAV vector comprises VP1, VP2 and/or VP3 capsid protein having 60% or more sequence identity to VP1, VP2 and/or VP3 capsid protein selected from the group consisting of AAV1, AAV2, AAV3, AAV4, AAV5, AAV6, AAV7, AAV8, AAV9, AAV10, AAV3B, AAV-2i8, Rh10, Rh74, SEQ ID NO:1 and SEQ ID NO:2 VP1, VP2 and/or VP3 capsid proteins.

In certain embodiments, the AAV vector comprises VP1, VP2 and/or VP3 capsid protein having 100% sequence identity to VP1, VP2 and/or VP3 capsid protein selected from the group consisting of AAV1, AAV2, AAV3, AAV4, AAV5, AAV6, AAV7, AAV8, AAV9, AAV10, AAV3B, AAV-2i8, Rh10, Rh74, SEQ ID NO:1 and SEQ ID NO:2 VP1, VP2 and/or VP3 capsid proteins.

In certain embodiments, the subject has antibodies or IgG that bind to the viral vector.

In certain embodiments, the antibodies or IgG that bind to the viral vector are absent from the subject.

In certain embodiments, the subject has antibodies or IgG that bind to the polypeptide or peptide encoded by the heterologous polynucleotide.

In certain embodiments, the antibodies comprise IgG, IgM, IgA, IgD and/or IgE.

In certain embodiments, a method includes determining the presence of, quantifying the amount of or an effector function of viral vector binding antibodies or IgG present in the subject before performing step (a), after performing step (a) but before performing step (b) and/or after performing steps (a) and (b).

In certain embodiments, a method includes analyzing a biological sample from the subject for the presence, amount or an effector function of viral vector binding antibodies or IgG present in the sample before performing step (a), after performing step (a) but before performing step (b) and/or after performing steps (a) and (b).

In certain embodiments, the determining and/or analyzing step is carried out before and/or after administration of the agent that decreases interaction of IgG with FcRn or the protease or glycosidase.

In certain embodiments, the biological sample from the subject is a blood product.

In certain embodiments, the method leads to a reduction of 20-50%, 50-75%, 75-90%, 90-95% or 95% or more of the viral vector binding antibodies or IgG.

In certain embodiments, the viral vector binding antibodies or IgG present in the biological sample or blood product from the subject is less than about 1:100,000 where 1 part of the biological sample or blood product diluted in 100,000 parts of buffer results in 50% viral vector neutralization.

In certain embodiments, the viral vector binding antibodies or IgG present in the biological sample or blood product from the subject is less than about 1:50,000, where 1 part of the biological sample or blood product diluted in 50,000 parts of buffer results in 50% viral vector neutralization.

In certain embodiments, the viral vector binding antibodies or IgG present in the biological sample or blood product from the subject is less than about 1:10,000, where 1 part of the biological sample or blood product diluted in 10,000 parts of buffer results in 50% viral vector neutralization.

In certain embodiments, the viral vector binding antibodies or IgG present in the biological sample or blood product from the subject is less than about 1:1,000, where 1 part of the biological sample or blood product diluted in 1,000 parts of buffer results in 50% viral vector neutralization.

In certain embodiments, the viral vector binding antibodies or IgG present in the biological sample or blood product from the subject is less than about 1:100, where 1 part of the biological sample or blood product diluted in 100 parts of buffer results in 50% viral vector neutralization.

In certain embodiments, the viral vector binding antibodies or IgG present in the biological sample or blood product from the subject is less than about 1:10, where 1 part of the biological sample or blood product diluted in 10 parts of buffer results in 50% viral vector neutralization.

In certain embodiments, the viral vector binding antibodies or IgG present in the biological sample or blood product is less than about 1:5, where 1 part of the biological sample or blood product diluted in 5 parts of buffer results in 50% viral vector neutralization.

In certain embodiments, the ratio of viral vector binding antibodies or IgG present in the biological sample or blood product is less than about 1:4, where 1 part of the biological sample or blood product diluted in 4 parts of buffer results in 50% viral vector neutralization.

In certain embodiments, the ratio of viral vector binding antibodies or IgG present in the biological sample or blood product is less than about 1:3, where 1 part of the biological sample or blood product diluted in 3 parts of buffer results in 50% viral vector neutralization.

In certain embodiments, the ratio of viral vector binding antibodies or IgG present in the subject, biological sample or blood product is less than about 1:2, where 1 part of the biological sample or blood product diluted in 2 parts of buffer results in 50% viral vector neutralization.

In certain embodiments, the ratio of viral vector binding antibodies or IgG present in the subject, biological sample or blood product is less than about 1:1, where 1 part of the biological sample or blood product diluted in 1 part of buffer results in 50% viral vector neutralization.

In certain embodiments, the method further comprising determining the presence of or quantifying the amount of antibodies or IgG that bind to a polypeptide or peptide encoded by the heterologous polynucleotide, after performing step (a) but before performing step (b) and/or after performing steps (a) and (b).

In certain embodiments, the method further comprising determining the presence of or quantifying the amount of antibodies or IgG that bind to the heterologous polynucleotide or nucleic acid after performing step (a) but before performing step (b) and/or after performing steps (a) and (b).

Methods according to the invention may also include, the use of a protease or glycosidase effective to degrade or digest and/or inhibit or reduce effector function of antibodies in combination with an agent that reduces the interaction of IgG with FcRn.

In certain embodiments, a subject has a lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, a lysosomal storage disease (e.g., aspartylglucosaminuria, Batten disease, late infantile neuronal ceroid lipofuscinosis type 2 (CLN2), cystinosis, Fabry disease, Gaucher disease types I, II, and III, glycogen storage disease II (Pompe disease), GM2-gangliosidosis type I (Tay Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A, B, C, D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), a disease of solid organs (e.g., brain, liver, kidney, heart), or an infectious viral (e.g., hepatitis B and C, HIV, etc.), bacterial or fungal disease. In certain embodiments, a subject has a blood clotting disorder. In certain embodiments, a subject has hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

In certain embodiments, a subject has anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors), or a platelet disorder such as, Bernard Soulier syndrome, Glanzmann thrombasthenia, or storage pool deficiency.

In certain embodiments, a subject has a disease that affects or originates in the central nervous system (CNS). In certain embodiments, the disease is a neurodegenerative disease. In certain embodiments, the CNS or neurodegenerative disease is Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain embodiments, the CNS or neurodegenerative disease is a polyglutamine repeat disease. In certain embodiments, the polyglutamine repeat disease is a spinocerebellar ataxia (SCA1, SCA2, SCA3, SCA6, SCA7, or SCA17).

In certain embodiments, the heterologous polynucleotide encodes a protein selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In certain embodiments, the heterologous polynucleotide encodes a protein selected from the group consisting of thrombopoietin (TPO), interleukins (IL1 through IL-36), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In certain embodiments, the heterologous polynucleotide encodes CFTR (cystic fibrosis transmembrane regulator protein), a blood coagulation (clotting) factor (Factor XIII, Factor IX, Factor VIII, Factor X, Factor VII, Factor VIIa, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, a sulfatase, N-acetylglucosamine-1-phosphate transferase, cathepsin A, GM2-AP, NPC1, VPC2, a sphingolipid activator protein, one or more zinc finger nuclease for genome editing, and one or more donor sequence used as repair templates for genome editing.

In certain embodiments, the heterologous polynucleotide encodes an inhibitory nucleic acid. In certain embodiments, the inhibitory nucleic acid is selected from the group consisting of a siRNA, an antisense molecule, miRNA, RNAi, a ribozyme and a shRNA. In certain embodiments, the inhibitory nucleic acid binds to a gene, a transcript of a gene, or a transcript of a gene associated with a polynucleotide repeat disease selected from the group consisting of a huntingtin (HTT) gene, a gene associated with dentatorubropallidoluysian atrophy (atrophin 1, ATN1), androgen receptor on the X chromosome in spinobulbar muscular atrophy, human Ataxin-1, -2, -3, and -7, Caᵥ2.1 P/Q voltage-dependent calcium channel (CACNA1A), TATA-binding protein, Ataxin 8 opposite strand (ATXN8OS), Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DNA damage-inducible transcript 4 protein, in diabetic macular edema (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; and mutant rhodopsin gene (RHO) in autosomal dominantly inherited retinitis pigmentosa (adRP).

In certain embodiments, the protein encoded by the heterologous polynucleotide comprises a gene editing nuclease. In certain embodiments, the gene editing nuclease comprises a zinc finger nuclease (ZFN) or a transcription activator-like effector nuclease (TALEN). In certain embodiments, the gene editing nuclease comprises a functional Type II CRISPR-Cas9.

In certain embodiments, step (a) and/or step (b) of a method of the invention are performed two or more times.

In certain embodiments, the subject is a mammal. In certain embodiments, the subject is a human.

Also disclosed herein are compositions, for example and without limitation, packages and kits, having components that may be used to practice methods according to the invention.

In certain embodiments, a package or kit has disposed therein: (a) a recombinant viral vector comprising a heterologous polynucleotide that encodes a protein or peptide; (b) an agent that reduces interaction of IgG with FcRn; (c) optionally, a protease or glycosidase that degrades or digests antibodies; and (d) a label with instructions for performing a method as disclosed herein. In certain embodiments, (a) (b) and (c) are in separate or the same container.

In certain embodiments, a package or kit has disposed therein: (a) a recombinant viral vector comprising a heterologous polynucleotide that is transcribed into a nucleic acid that inhibits, decreases or reduces expression of a protein; (b) an agent that reduces interaction of IgG with FcRn; (c) optionally, a protease or glycosidase that degrades or digests antibodies; and (d) a label with instructions for performing a method as disclosed herein. In certain embodiments, (a) (b) and (c) are in separate or the same container.

### DESCRIPTION OF DRAWINGS

FIGS. 1A, 1B and 1C show SDS-PAGE analyses of cleavage of IgG by IdeS in samples of human patient sera (FIG. 1A), non-human primate (rhesus macaque) plasma (FIG. 1B) and hamster plasma (FIG. 1C), incubated with increasing amounts of IdeS. Samples were incubated without IdeS or with increasing concentrations of IdeS for 1 hr at 37° C. The reactions were stopped by addition of sample buffer. Samples were analyzed by non-reducing SDS-PAGE and Coomassie stain.

FIG. 2 is a graph showing GAA activity levels in murine plasma after infusion of AAV-Spk1-GAA vector in animals immunized with varying amounts of IVIg that was pre-treated with or without IdeZ. AAV-Spk1-GAA vector was infused one day after IVIg immunization. Transgene activity was assessed by GAA Activity Assay at 2 weeks post vector administration. GAA activity in nmol/hr/mL is plotted for each mouse in each group. Control mice were administered only vector in the absence of IVIg.

FIG. 3 shows anti-Spk1 neutralizing antibody (NAb) titer levels in murine plasma pre- and post-IdeS infusion. Relative NAb titer levels in this study are designated as low titer (<1:1, 1:1-1:2.5) (bold), mid-range (1:2.5-1:5) (bold italics), and high (>1:5-1:10) (italics).

FIG. 4 shows anti-Spk1 IgG NAb levels (ng/mL) in murine plasma pre- and post-IdeS infusion. Negative control animals were not treated with either IVIg or IdeS. IdeS Low refers to 0.4 mg/kg IdeS used in the study, and IdeS High refers to 4 mg/kg IdeS.

FIG. 5 shows GAA activity levels (nmol/hr/mL) in murine plasma after infusion of AAV-Spk1-GAA vector in animals immunized with IVIg then treated with IdeS. All animals received 2×10¹² vg/kg AAV-Spk1-GAA vector. Transgene activity was measured in plasma samples from mice immunized with IVIg, then treated with or without IdeS, and finally administered with vector. Transgene activity was assessed by GAA Activity Assay at 1 week post vector administration. GAA activity in nmol/hr/mL is plotted for each mouse in each group.

FIG. 6 shows GAA activity levels (nmol/hr/mL) two weeks after infusion of AAV-Spk1-GAA vector (2×10¹² vg/kg) in animals previously infused with IVIg (0, 300, 800, or 1600 mg/kg) and treated with IdeS (0, 0.4, 1.0 or 2.0 mg/kg). GAA activity is plotted for each mouse in each group. Mice in IVIg administered groups that did not develop a corresponding anti-Spk1 NAb titer (i.e., having NAb titer <1:1 or 1:1-1:2.5 pre-IdeS treatment) were excluded.

FIG. 7 shows anti-Spk1 NAb titer levels in the plasma of C57BL/6 mice, having an artificial titer of human anti-capsid neutralizing IgG, measured pre- and post-infusion with different preparations of IdeS (Lot #1 and Lot #2).

FIG. 8 is a graph showing levels of human Factor VIII in plasma from C57BL/6 mice, having an artificial titer of human anti-capsid neutralizing IgG, pre-dose and at 1 and 2 weeks after dosing with AAV-Spk1-hFVIII.

FIG. 9 is a graph showing anti-Spk1 capsid IgG levels (ng/mL) in mouse plasma pre- and post-IdeS infusion. C57BL/6 mice were given IVIg to induce an artificial titer of human anti-capsid neutralizing IgG. Negative control animals were not treated with either IVIg or IdeS. Low IVIg refers to 300 mg/kg IVIg used in the study, Mid IVIg refers to 800 mg/kg, and High IVIg refers to 1600 mg/kg. Within each IVIg group, animals were treated with increasing doses of IdeS (0, 0.4, 1.0, 2.0 mg/kg IdeS). Animals treated with IVIg but demonstrating no anti-capsid IgG response were excluded from the graph.

FIG. 10 is a graph of NAb titers assessed in mouse plasma on Day 0 (DO) and Day 2 (D2) for each of the five groups of Tg32 mice of the study described in Example 13. Upper and lower dotted line denote the upper limit of detection (ULOD) and lower limit of detection (LLOD), respectively. Statistical significance is defined as p<0.0234=*, p<0.0039=** by a Wilcoxin paired nonparametric t-test, and p<0.5 by a Mann-Whitney unpaired nonparametric t-test.

FIG. 11 is a graph of anti-Spk1 IgG concentration, as determined by ELISA, in plasma of mice at day 0 (D0) and day 2 (D2) for each of five groups of Tg32 mice of the study described in Example 13. Anti-Spk1 IgG ELISA. Statistical significance is defined as p<0.0313=** by a Wilcoxin paired nonparametric t-test, and p<0.0152=*, p<0.0022=** by a Mann-Whitney unpaired nonparametric t-test.

FIG. 12 presents graphical representations of the concept of combining administration of an anti-FcRn agent and IdeS to eliminate high titer neutralizing antibodies (NAbs) in a subject to enable AAV transduction.

FIG. 13 schematically shows a redosing study in New Zealand white rabbits.

FIG. 14 schematically shows an anti-FcRn study in cynomolgus monkeys.

### DETAILED DESCRIPTION

Provided herein are methods to improve the benefit or effectiveness of gene therapy comprising administration of an agent that that inhibits or reduces the interaction of IgG with the neonatal Fc receptor (FcRn). Also provided herein are methods to decrease the circulating antibodies that bind to a viral vector, such as a recombinant viral vector, or that bind to a nucleic acid or a polypeptide, protein or peptide encoded by a therapeutic heterologous polynucleotide encapsidated by a recombinant viral vector, or that bind to the therapeutic heterologous polynucleotide. Also provided herein are methods to administer a gene therapy vector to a subject having antibodies that bind and/or neutralize the gene therapy vector. Also provided herein are methods to re-dose or re-administer a gene therapy vector to a subject to whom a gene therapy vector was previously administered, and wherein the subject has developed antibodies that bind and/or neutralize the gene therapy vector.

In certain embodiments, a method comprises administering to a subject an amount of an agent effective to reduce the interaction of IgG with FcRn. The FcRn is responsible for extending the serum half-life of immunoglobulins of the IgG type through a process referred to as IgG recycling. FcRn is located in the endosomal compartment of many cell types. When endocytosed IgG moves through the endosomal pathway, the Fc portion of the IgG is bound by FcRn in the acidic, early endosome to form an FcRn-IgG complex. The FcRn-IgG complex is trafficked away from the lysosomal pathway (which would normally degrade or catabolize the IgG) and back to the plasma membrane and cell surface. The elevated extracellular pH results in dissociation of the complex and release of IgG out of the cell and back into the circulation, thus extending the serum half-life of IgG. Reducing or inhibiting the interaction of IgG with FcRn will decrease IgG recycling, increase or enhance IgG clearance, degradation and catabolism, and result in lower amounts (reduced titer) of IgG in the circulation (as measurable in blood, serum or plasma). Lower titers of circulating IgG means lower titers of circulating antibodies that bind and/or neutralize a gene therapy vector. Administration of an agent that can inhibit or reduce the interaction between IgG and FcRn will decrease IgG recycling, increase or enhance IgG clearance, degradation and catabolism, and will result in lower amounts (reduced titers) of IgG in the circulation, and improved or enhanced efficacy of gene therapy treatments.

As used herein, “reduce the interaction of IgG with FcRn” and “reduce interaction of IgG with FcRn” includes any decrease or inhibition in the interaction of IgG with FcRn or IgG-FcRn binding. Agents that reduce the interaction of IgG with FcRn and that may be used in the invention are reviewed in Low et al., 2009, AAPS J., 11:432-434, Sockolosky et al., 2015, Adv. Drug Deliv. Rev., 91:109-124, Zuercher et al., 2019, Autoimm. Rev., doi.org/10.1016/j.autrev.2019.102366, and Pyzik et al., 2019, Frontiers Immunol., doi: 103389/fimmu.2019.01540. Reduced, decreased or inhibited interaction of IgG with FcRn can be assessed by detecting or measuring one or more signaling activities or downstream readouts of FcRn activity, including serum or plasma levels of IgG. An agent that reduces interaction of IgG with FcRn is also referred to herein as an “anti-FcRn agent” or an “FcRn antagonist.”

Examples of agents that reduce the interaction of IgG and FcRn that may be used in the invention include, for example and without limitation, antibodies that bind FcRn, ABDEGs (antibodies that enhance IgG degradation), FcRn-binding peptides (including those having a GHFGGXY (SEQ ID NO: 57) consensus motif, where X is a hydrophobic amino acid and the motif is enclosed by a disulfide loop), FcRn-binding affibodies, and small molecule FcRn antagonists.

An example of an anti-FcRn antibody that may be used in the invention is M281 (nipocalimab), a fully human, anti-FcRn antibody that inhibits FcRn-mediated recycling and decreases pathogenic IgG, while preserving IgG production (see Ling et al. 2019). Other anti-FcRn antibodies that may be used in the invention include, for example and without limitation, antibodies described in International Patent Application publications WO2018023136, WO2019118791 and WO2020018910, each of which is incorporated herein in its entirety by reference, including all text, tables, sequence listings and drawings.

In certain embodiments, the anti-FcRn antibody comprises a light chain and a heavy chain, and the light chain comprises a sequence having at least 90%, at least 91%, at least 92%, at least 93%, at least 94%, at least 95%, at least 96%, at least 97%, at least 98%, at least 99%, or 100% identity to the sequence of SEQ ID NO:49; and the heavy chain comprises a sequence having at least 90%, at least 91%, at least 92%, at least 93%, at least 94%, at least 95%, at least 96%, at least 97%, at least 98%, at least 99%, or 100% identity to the sequence of SEQ ID NO:50

In certain embodiments, the anti-FcRn antibody comprises a light chain and/or a heavy chain, and the light chain comprises a sequence having at least 90%, at least 91%, at least 92%, at least 93%, at least 94%, at least 95%, at least 96%, at least 97%, at least 98%, at least 99%, or 100% identity to the sequence of SEQ ID NO:49; and the heavy chain comprises a sequence having at least 90%, at least 91%, at least 92%, at least 93%, at least 94%, at least 95%, at least 96%, at least 97%, at least 98%, at least 99%, or 100% identity to the sequence of SEQ ID NO:50.

In certain embodiments, the anti-FcRn antibody comprises a light chain and/or a heavy chain, and the light chain comprises at least one complemtarity determining region (CDR) sequence selected from the group consisting of SEQ ID NO:51, SEQ ID NO:52 and SEQ ID NO:53; and the heavy chain comprises at least one CDR sequence selected from the group consisting of SEQ ID NO:54, SEQ ID NO:55 and SEQ ID NO:56.

In certain embodiments, the anti-FcRn antibody comprises a light chain and/or a heavy chain, and the light chain comprises the complemtarity determining region (CDR) sequences of SEQ ID NO:51, SEQ ID NO:52 and SEQ ID NO:53; and the heavy chain comprises the CDR sequences of SEQ ID NO:54, SEQ ID NO:55 and SEQ ID NO:56.

In certain embodiments, the anti-FcRn antibody comprises a light chain comprising a sequence having at least 90%, at least 91%, at least 92%, at least 93%, at least 94%, at least 95%, at least 96%, at least 97%, at least 98%, at least 99%, or 100% identity to the sequence of SEQ ID NO:49.

In certain embodiments, the anti-FcRn antibody comprises a light chain comprising the sequence of SEQ ID NO:49.

In certain embodiments, the anti-FcRn antibody comprises a heavy chain comprising a sequence having at least 90%, at least 91%, at least 92%, at least 93%, at least 94%, at least 95%, at least 96%, at least 97%, at least 98%, at least 99%, or 100% identity to the sequence of SEQ ID NO:50.

In certain embodiments, the anti-FcRn antibody comprises a heavy chain comprising the sequence of SEQ ID NO:50.

An example of an FcRn-binding peptide that may be used in the invention is SYN1436, which binds FcRn and decreases overall serum IgG levels (see Mezo et al., 2008, Bioorg. Med. Chem., 16:6394-6405).

Affibody molecules are affinity protein domains, 58 amino acids long, having a folded anti-parallel three-helix bundle structure. Anti-FcRn affibodies that may be used in the invention include, for example and without limitation, ZFcRn and others described in Seijseng et al., 2018, Scientific Reports, 8:5141, doi:10.1038/s41598-018-23481-5.

Examples of small molecule FcRn antagonists that decrease interaction of IgG with FcRn and that may be used in the invention are described in Wang et al., 2013, Bioorg. Med. Chem. Let., 23:1253-1256.

ABDEGs are IgG molecules where the Fc-portion is engineered to bind with high affinity to FcRn at both physiological and endosomal pH.

Examples of FcRn antagonist compositions having variant Fc regions that bind to FcRn with increased affinity and that may be used in the invention are described in international patent application publication WO2015/100299.

Efgartigimod, is a modified (by ABDEG technology) human IgG1-derived Fc fragment that binds FcRn with high affinity, and prevents FcRn from interacting with and recycling circulating IgGs (Ulrichts et al., 2018, J. Clin. Invest., 128:4372-4386).

Rozanolixizumab is an IgG4P isotype anti-FcRn monoclonal antibody that reduces IgG levels in humans by about 45% when administered at a dose of 7 mg/kg (Kiessling et al., 2017, Sci. Transl. Med., 9:aan1208. doi: 10.1126/scitranslmed.aan1208).

SYNT001 is an FcRn-blocking monoclonal antibody that decreases all circulating IgG subtypes and IgG immune complexes in humans (Blumberg et al., 2019, Sci. Adv., 5:eaax9586).

In certain embodiments, the agent that reduces interaction of IgG with FcRn inhibits signaling mediated by interaction between FcRn and IgG.

In certain embodiments the agent that reduces interaction of IgG with FcRn is a peptide, protein, small molecule, nucleic acid, aptamer, oligonucleotide, affibody, antibody or a combination thereof.

In certain embodiments, the agent that reduces interaction of IgG with FcRn is selected from efgartigimod, M281, rozanolixizumab, SYNT001 and IMVT-1401.

In certain embodiments, the agent that reduces interaction of IgG with FcRn is an antibody selected from the group consisting of monoclonal antibody or a fragment thereof, a polyclonal antibody or a fragment thereof, chimeric antibody, humanized antibody and single chain antibody.

In certain embodiments, the agent that reduces interaction of IgG with FcRn is a bispecific agent comprising binding sites for IgG and FcRn.

In certain embodiments, the agent that reduces interaction of IgG with FcRn is a recombinant Fc portion of IgG or a biologically active portion thereof or a proteo-mimetic thereof

In certain embodiments, circulating IgG is reduced by at least 10%, at least 20%, at least 30%, at least 40%, at least 50%, at least 60%, at least 70%, at least 80%, at least 85%, at least 90%, at least 95%, at least 99%, or 100%.

Further provided herein is a composition provided, for example, as a package or kit having (a) a recombinant viral vector comprising a heterologous polynucleotide that encodes a protein or peptide or a nucleic acid; (b) an agent that reduces interaction of IgG with FcRn, (c) optionally, a protease or glycosidase that degrades or digests antibodies; and (d) a label with instructions for performing the method described herein, wherein (a), (b) and (c) are provided in separate or the same container(s).

In certain embodiments, a method additionally comprises administering to a subject an amount of a glycosidase effective to inhibit or reduce effector function of antibodies that bind to a recombinant viral vector, and/or a nucleic acid, and/or a protein or peptide encoded by a heterologous polynucleotide. In certain embodiments, a method additionally comprises administering to a subject an amount of an endopeptidase effective to degrade or digest antibodies or an endoglycosidase effective to inhibit or reduce effector function of antibodies that bind to a recombinant viral vector, and/or a nucleic acid, and/or a protein or peptide encoded by a heterologous polynucleotide.

In certain embodiments, a method additionally comprises administering to a subject an amount of a glycosidase effective to reduce Fc receptor binding of antibodies that bind to a recombinant viral vector, and/or a nucleic acid, and/or a protein or peptide encoded by a heterologous polynucleotide. In certain embodiments, a method additionally comprises administering to a subject an amount of an endoglycosidase effective to reduce Fc receptor binding of antibodies that bind to a recombinant viral vector, and/or a nucleic acid, and/or a protein or peptide encoded by a heterologous polynucleotide.

The methods of the invention are widely applicable to the enhancement of gene therapy treatments. For example, overcoming NAbs to the AAV capsid or other mode of gene therapy delivery by administration of an agent that reduces interaction of IgG with FcRn has the potential to enable treatment of patients with pre-existing AAV neutralizing antibody titers, as well as enable repeat dosing of patients previously administered with an AAV gene therapy product where effective levels have either not been achieved or have been lost due to time or other confounding issue. Administration of an agent that reduces interaction of IgG with FcRn in combination with administration of IdeS, for example, may further enhance the efficacy of gene therapy treatment of patients with pre-existing NAbs. Additionally, the methods herein enable hepatic gene transfer to the pediatric population, which has been seen as intractable to gene therapy due to hepatocyte expansion and potential loss of transgene expression during development. In a further example, in methods of the invention, administration of an agent that reduces interaction of IgG with FcRn, and, optionally in combination with administration of a protease (e.g., IdeS) or a glycosidase (e.g., EndoS) will reduce or clear neutralizing antibodies against the AAV capsid and enable treatment of patients previously viewed as not eligible for gene therapy or that develop AAV antibodies after AAV gene therapy.

In certain embodiments, viral vectors that may be used in the invention include, for example and without limitation, AAV particles. In certain embodiments, viral vectors that may be used in the invention include, for example and without limitation, retroviral, adenoviral, helper-dependent adenoviral, hybrid adenoviral, herpes simplex virus, lentiviral, poxvirus, Epstein-Barr virus, vaccinia virus, and human cytomegalovirus vectors, including recombinant versions thereof.

The term “recombinant,” as a modifier of a viral vector, such as a recombinant AAV (rAAV) vector, as well as a modifier of sequences such as recombinant polynucleotides and polypeptides, means that compositions have been manipulated (i.e., engineered) in a fashion that generally does not occur in nature. A particular example of a recombinant AAV vector would be where a nucleic acid that is not normally present in a wild-type AAV genome (heterologous polynucleotide) is inserted within a viral genome. An example of which would be where a nucleic acid (e.g., gene) encoding a therapeutic protein or polynucleotide sequence is cloned into a vector, with or without 5′, 3′ and/or intron regions that the gene is normally associated within the AAV genome. Although the term “recombinant” is not always used herein in reference to an AAV vector, as well as sequences such as polynucleotides, recombinant forms including AAV vectors, polynucleotides, etc., are expressly included in spite of any such omission.

A “rAAV vector,” for example, is derived from a wild-type genome of AAV by using molecular methods to remove all or a part of a wild-type AAV genome, and replacing with a non-native (heterologous) nucleic acid, such as a nucleic acid encoding a therapeutic protein or polynucleotide sequence. Typically, for a rAAV vector one or both inverted terminal repeat (ITR) sequences of AAV genome are retained. A rAAV is distinguished from an AAV genome since all or a part of an AAV genome has been replaced with a non-native sequence with respect to the AAV genomic nucleic acid, such as with a heterologous nucleic acid encoding a therapeutic protein or polynucleotide sequence. Incorporation of a non-native (heterologous) sequence therefore defines an AAV as a “recombinant” AAV vector, which can be referred to as a “rAAV vector.”

A recombinant AAV vector sequence can be packaged-referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV,” “rAAV particle” and/or “rAAV virion.” Such rAAV, rAAV particles and rAAV virions include proteins that encapsidate or package a vector genome. Particular examples include in the case of AAV, capsid proteins.

A “vector genome,” which may be abbreviated as “vg,” refers to the portion of the recombinant plasmid sequence that is ultimately packaged or encapsidated to form a rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant AAV vector production, but is not itself packaged or encapsidated into rAAV particles. Thus, a “vector genome” refers to the nucleic acid that is packaged or encapsidated by rAAV.

As used herein, the term “serotype” in reference to an AAV vector means a capsid that is serologically distinct from other AAV serotypes. Serologic distinctiveness is determined on the basis of lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes). An antibody to one AAV may cross-react with one or more other AAV serotypes due to homology of capsid protein sequence.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors include any viral strain or serotype. For example and without limitation, a rAAV vector genome or particle (capsid, such as VP1, VP2 and/or VP3) can be based upon any AAV serotype, such as AAV-1, -2, -3, -4, -5, -6, -7, -8, -9, -10, -11, -12, -rh74, -rh10, AAV3B or AAV-2i8, for example. Such vectors can be based on the same strain or serotype (or subgroup or variant), or be different from each other. For example and without limitation, a rAAV plasmid or vector genome or particle (capsid) based upon one serotype genome can be identical to one or more of the capsid proteins that package the vector. In addition, a rAAV plasmid or vector genome can be based upon an AAV serotype genome distinct from one or more of the capsid proteins that package the vector genome, in which case at least one of the three capsid proteins could be a different AAV serotype, e.g., AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2), or variant thereof, for example. More specifically, a rAAV2 vector genome can comprise AAV2 ITRs but capsids from a different serotype, such as AAV1, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2), or variant thereof, for example. Accordingly, rAAV vectors include gene/protein sequences identical to gene/protein sequences characteristic for a particular serotype, as well as “mixed” serotypes, which also can be referred to as “pseudotypes.”

In certain embodiments, the rAAV plasmid or vector genome or particle is based upon reptile or invertebrate AAV variants, such as snake and lizard parvovirus (Pénzes et al., 2015, J. Gen. Virol., 96:2769-2779) or insect and shrimp parvovirus (Roekring et al., 2002, Virus Res., 87:79-87).

In certain embodiments, the recombinant plasmid or vector genome or particle is based upon a bocavirus variant. Human bocavirus variants are described, for example, in Guido et al., 2016, World J. Gastroenterol., 22:8684-8697.

In certain embodiments, a rAAV vector includes or consists of a capsid sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2) capsid proteins (VP1, VP2, and/or VP3 sequences). In certain embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rh10 or AAV3B, ITR(s).

In certain embodiments, rAAV vectors include AAV1, AAV2, AAV3, AAV4, AAV5, AAV6, AAV7, AAV8, AAV9, AAV10, AAV11, AAV12, AAV3B, Rh10, Rh74 and AAV-2i8 variants (e.g., ITR and capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773).

rAAV, such as AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2) and variants, hybrids and chimeric sequences, can be constructed using recombinant techniques that are known to a skilled artisan, to include one or more heterologous polynucleotide sequences (transgenes) flanked with one or more functional AAV ITR sequences. Such AAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

In certain embodiments, a lentivirus used in the invention may be a human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), or caprine arthritis encephalitis virus (CAEV). Lentiviral vectors are capable of providing efficient delivery, integration and long-term expression of heterologous polynucleotide sequences into non-dividing cells both in vitro and in vivo. A variety of lentiviral vectors are known in the art, see Naldini et al. (Proc. Natl. Acad. Sci. USA, 93:11382-11388 (1996); Science, 272: 263-267 (1996)), Zufferey et al., (Nat. Biotechnol., 15:871-875, 1997), Dull et al., (J Virol. 1998 November; 72(11):8463-71, 1998), U.S. Pat. Nos. 6,013,516 and 5,994,136, any of which may be a suitable viral vector for use in the invention.

An immune response, such as humoral immunity, can develop against a wild-type virus in a subject exposed to the wild-type virus. Such exposure can lead to pre-existing antibodies in the subject that bind to a viral vector based upon the wild-type virus even prior to treatment with a gene therapy method employing the viral vector.

An immune response, such as humoral immunity, can also develop against a recombinant viral vector, and/or a heterologous polynucleotide or a protein or peptide encoded by a heterologous polynucleotide encapsidated by the viral vector, resulting in inhibition or reduction in viral vector cell transduction, heterologous polynucleotide expression or function, or function or activity of the protein or peptide encoded by a heterologous polynucleotide in a subject to which the viral vector is administered.

Antibodies that bind to a viral vector used in the invention, such as a recombinant viral vector, which can be referred to as “neutralizing” antibodies, can reduce or inhibit cell transduction of viral vectors useful for gene therapy. As a result, while not being bound by theory, cell transduction is reduced or inhibited thereby reducing introduction of the viral packaged heterologous polynucleotide into cells and subsequent expression and, as appropriate, subsequent translation into a protein or peptide. Additionally, antibodies that bind to a heterologous polynucleotide or a protein or peptide encoded by a heterologous polynucleotide encapsidated by the viral vector can inhibit expression of a heterologous polynucleotide, function or activity of a heterologous polynucleotide or function or activity of a protein or peptide encoded by a heterologous polynucleotide.

Accordingly, antibodies can be present that bind to a recombinant viral vector (e.g., AAV) and/or antibodies can be present that bind to a protein or peptide encoded by a heterologous polynucleotide in a subject. In addition, antibodies can be present that bind to a heterologous polynucleotide encapsidated by the recombinant viral vector.

IgG antibodies that bind to a recombinant viral vector (e.g., AAV) or that bind to a protein or peptide encoded by a heterologous polynucleotide, should they be produced, can be reduced in a subject by use of an agent that reduces the interaction of IgG with FcRn as set forth herein. Reduction of the interaction of IgG with FcRn results in reduced IgG recycling (also referred to as enhanced clearance of IgG), and thus reduced titer of circulating IgG (reduced levels of IgG in blood, plasma or serum), which can be measured by standard assays known in the art.

Antibodies that bind to a recombinant viral vector (e.g., AAV) or that bind to a protein or peptide encoded by a heterologous polynucleotide, should they be produced, can additionally be degraded or digested by a protease as set forth herein. Antibodies that bind to a recombinant viral vector (e.g., AAV) or that bind to a protein or peptide encoded by a heterologous polynucleotide, should they be produced, can also have their effector function reduced or inhibited as set forth herein.

As used herein, “effector function” in reference to an antibody means normal functional attributes of an antibody. Nonlimiting examples of antibody functional attributes include, for example, binding to an antigen; activation of the complement cascade (referred to as complement dependent cytotoxicity); binding to Fc receptor on effector cells, such as macrophages, monocytes, natural killer cells and eosinophils, to engage antibody—dependent cellular cytotoxicity (ADCC); and as a signal for ingestion of bound antigen/pathogen by immune cells such as phagocytes and dendritic cells. A reduction or inhibition of antibody effector function can therefore refer to any one or more of the foregoing nonlimiting functional attributes. Effector function assays are known in the art as well as described in WO2016012285, for example.

An “Fc receptor” refers to any Fc receptor. In addition to the neonatal Fc receptor (FcRn), nonlimiting examples of Fc receptors include Fe gamma immunoglobulin receptors (FcγRs) which are present on cells. In humans, FcγR refers to one, some, or all of the family of Fc receptors comprising FcγRI (CD64), FcγRIIA (CD32A), FcγRIIB (CD32B), FcγRIIIA (CD16a) and FcγRIIIB (CD16b). FcγR includes naturally occurring polymorphisms of FcγRI (CD64), FcγRIIA (CD32A), FcγRIIB (CD32B), FcγRIIIA (CD16a) and FcγRIIIB (CD16b).

In certain embodiments, antibody binding to a viral vector is reduced or inhibited by way of an agent that reduces interaction of IgG with FcRn, a protease or a glycosidase.

In certain embodiments, antibody binding to Fc receptor on effector cells, such as macrophages, monocytes, natural killer cells or eosinophils, is reduced or inhibited by way of a glycosidase. In certain embodiments, an endoglycosidase hydrolyzes a glycan structure on the Fe interacting domain of an antibody. In certain embodiments, an endoglycosidase hydrolyzes a glycan structure on the Fe interacting domain of an IgG, such as the N-linked bi-antennary glycan at position Asn-297 (Kabat numbering).

In certain embodiments, antibody activation of complement cascade is reduced or inhibited by way of an agent that reduces interaction of IgG with FcRn, a protease or glycosidase.

In certain embodiments, antibody stimulation or reduction of ingestion by immune cells such as phagocytes or dendritic cells, is reduced or inhibited by way of an agent that reduces interaction of IgG with FcRn, a protease or glycosidase.

In certain embodiments, an agent that reduces interaction of IgG with FcRn is administered to a subject before administration of a recombinant viral (e.g., AAV) vector. In certain embodiments, an agent that reduces interaction of IgG with FcRn is administered to a subject after administration of a recombinant viral (e.g., AAV) vector. In certain embodiments, a recombinant viral (e.g., AAV) vector and an agent that reduces interaction of IgG with FcRn are administered substantially contemporaneously, or at about the same time.

In certain embodiments, in addition to an agent that reduces interaction of IgG with FcRn, a protease and/or glycosidase is administered to a subject before administration of a recombinant viral (e.g., AAV) vector. In certain embodiments, an agent that reduces interaction of IgG with FcRn is administered to a subject after administration of a protease and/or glycosidase, and/or after administration of a recombinant viral (e.g., AAV) vector. In certain embodiments, a recombinant viral (e.g., AAV) vector and an agent that reduces interaction of IgG with FcRn are administered substantially contemporaneously, or at about the same time as administration of an agent that reduces interaction of IgG with FcRn.

In certain embodiments, an agent that reduces interaction of IgG with FcRn, such as for example and without limitation, an anti-FcRn antibody, is administered to a subject prior to administration to the subject of an endopeptidase, such as and without limitation, an IdeS, followed by administration to the subject of a recombinant viral (e.g., AAV) vector. In certain embodiments, the agent that reduces interaction of IgG with FcRn, such as for example and without limitation, an anti-FcRn antibody, is administered to a subject more than one time prior to administration of the endopeptidase, such as and without limitation, an IdeS. In certain embodiments the agent that reduces interaction of IgG with FcRn, such as for example and without limitation, an anti-FcRn antibody, is administered to a subject at least two times, at least three times, at least 4 times or at least 5 times prior to administration of the endopeptidase, such as and without limitation, an IdeS.

In certain embodiments, an agent that reduces interaction of IgG with FcRn is administered to a subject over a period of 1, 2, 3, 4, 5, 6 or more weeks prior to administration of a protease and/or glycosidase, and after administration of the protease and/or glycosidase, a recombinant viral (e.g., AAV) vector is administered to the subject.

In certain embodiments, an agent that reduces interaction of IgG with FcRn is administered to a subject about 1 hour, about 2 hours, about 3 hours, about 4 hours, about 5 hours, about 6 hours, about 7 hours, about 8 hours, about 9 hours, about 10 hours, about 15 hours, about 20 hours, about one day, about 2 days, about 3 days, about 4 days, about 5 days, about 6 days or about one week prior to administration to the subject of a protease and/or glycosidase, and after the administration of the protease and/or glycosidase, a recombinant viral (e.g., AAV) vector is administered to the subject.

In certain embodiments, an agent that reduces interaction of IgG with FcRn, such as for example and without limitation, an anti-FcRn antibody, is administered to a subject over a period of 1, 2, 3, 4, 5, 6 or more weeks prior to administration to the subject of an endopeptidase, such as and without limitation, an IdeS, and after administration of the endopeptidase, a recombinant viral (e.g., AAV) vector is administered to the subject.

In certain embodiments, an agent that reduces interaction of IgG with FcRn, such as for example and without limitation, an anti-FcRn antibody, is administered to a subject about 1 hour, about 2 hours, about 3 hours, about 4 hours, about 5 hours, about 6 hours, about 7 hours, about 8 hours, about 9 hours, about 10 hours, about 15 hours, about 20 hours, about one day, about 2 days, about 3 days, about 4 days, about 5 days, about 6 days or about one week prior to administration to the subject of an endopeptidase, such as and without limitation, an IdeS, and after the administration of the endopeptidase, a recombinant viral (e.g., AAV) vector is administered to the subject.

Antibodies comprise any of IgG, IgM, IgA, IgD and/or IgE. Accordingly, in certain embodiments, the invention is directed to inter alia digesting, degrading or reducing or inhibiting effector function of any of one of these five classes of antibodies, any two of these five classes of antibodies, any three of these five classes of antibodies, any four of these five classes of antibodies or all five of these five classes of antibodies.

Levels of antibodies in a subject can be analyzed, measured or determined before and/or after administration of a recombinant viral vector. Levels of antibodies in a subject can also be analyzed, measured or determined before and/or after the administration of an agent that reduces interaction of IgG with FcRn or a protease or glycosidase. Levels of antibodies in a subject may also be analyzed or measured multiple times, before and/or after administration of a recombinant viral vector as well as before and/or after administration of an agent that reduces interaction of IgG with FcRn or a protease or glycosidase.

Effector function of antibodies in a subject can be analyzed, measured or determined before and/or after administration of a recombinant viral vector. Effector function of antibodies in a subject can also be analyzed, measured or determined before and/or after the administration of an agent that reduces interaction of IgG with FcRn or a protease or glycosidase. Effector function of antibodies in a subject may also be analyzed or measured multiple times, before and/or after administration of a recombinant viral vector as well as before and/or after administration of an agent that reduces interaction of IgG with FcRn or a protease or glycosidase.

An increase in the equilibrium binding constant corresponds to a decrease in the binding between IgG and an Fc receptor. Accordingly, a reduction in Fc receptor binding of an antibody as a consequence of activity of an agent that reduces interaction of IgG with FcRn or protease or glycosidase activity may result in an increase in the equilibrium binding constant for the IgG:FcR interaction. A reduction in Fc receptor binding of an antibody may result in an increase in the equilibrium binding constant for the IgG:FcR interaction by a factor of at least 1, at least 2, at least 3, or at least 4, or at least 5, or at least 6, or at least 7 or at least 8.

In certain embodiments, an immune response (e.g., a humoral immune response) in a subject caused by exposure to wild-type virus is treated by administering an agent that reduces interaction of IgG with FcRn and an amount of a protease and/or glycosidase effective to degrade or digest antibodies that bind to a recombinant viral vector based upon the wild-type virus prior to administration of the recombinant viral vector to the subject.

In certain embodiments, an immune response (e.g., a humoral immune response) caused by administration of a recombinant viral vector such as AAV is treated by administering an agent that reduces interaction of IgG with FcRn and an amount of a protease and/or glycosidase effective to degrade or digest antibodies that bind to the recombinant viral vector, or antibodies that bind to the heterologous polynucleotide or a protein or peptide encoded by the heterologous polynucleotide encapsidated by the viral vector.

In certain embodiments, administration of a recombinant viral vector to a subject is preceded by administration of an agent that reduces interaction of IgG with FcRn and a protease and/or glycosidase to inhibit or prevent an immune response (e.g., a humoral immune response) against the recombinant viral vector or antibodies that bind to the heterologous polynucleotide or a protein or peptide encoded by the heterologous polynucleotide encapsidated by the viral vector

In certain embodiments, an agent that reduces interaction of IgG with FcRn and a protease and/or glycosidase is administered to a subject before an immune response (e.g., a humoral immune response), such as before development of neutralizing antibodies or development of antibodies that bind to the heterologous polynucleotide or a protein or peptide encoded by the heterologous polynucleotide encapsidated by the viral vector.

The use of proteases and/or glycosidases in methods to increase or enhance the efficacy of gene therapy vectors and to increase or enhance gene therapy treatment in a subject is known. See for example WO2020016318, WO2020102740 and Leborgne et al., 2020, Nat. Med., 26:1096-1101 (2020), each of which is incorporated herein in its entirety by reference, including all text, tables, sequence listings and drawings.

Proteases are enzymes that degrade or digest proteins. As used herein, proteases are also designated peptidases, proteinases, peptide hydrolases, or proteolytic enzymes.

Proteases that may be used in the invention can be subdivided into two broad groups based on their substrate-specificity. Proteases may be of the exo-type that hydrolyzes peptide bonds located towards the N-terminal end or the C-terminal end (exoprotease or exopeptidase). Examples of exoproteases or exopeptidases, include, for example and without limitation, Flavozyme (Novozymes), ProteaAX (Amano), and Pancreatin from porcine pancreas.

Proteases that may be used in the invention may be of the endo-type that hydrolyzes peptide bonds internally in polypeptide chains (endoprotease or endopeptidase). Examples of endoproteases include, for example and without limitation, IdeS, IdeZ, IgdE, IdeMC, trypsin, chymotrypsin, papain and pepsin.

Examples of proteases that may be used in the invention include, for example and without limitation, cysteine proteases from Streptococcus pyogenes, Streptococcus equi, Mycoplasma canis, S. agalactiae or S. pseudoporcinus. In certain embodiments, a protease includes endopeptidase IdeS from Streptococcus pyogenes or a modified variant thereof set forth in any of SEQ ID NO:3-18. In certain embodiments, a protease includes a protease set forth in SEQ ID NO:19 or SEQ ID NO:20, or a modified variant thereof. In certain embodiments, a protease includes endopeptidase IdeZ from Streptococcus equi, or a modified variant thereof set forth in any of SEQ ID NOs:21-43.

Other proteases that may be used in the invention include, for example and without limitation, IgdE enzymes from S. suis, S. porcinus, S. equi, described in international patent application publication WO 2017/134274. Other proteases that may be used in the invention include, for example and without limitation, IdeMC and homologs described in international patent application publication WO 2018/093868. Other endopeptidases that may be used in the invention include, for example and without limitation, IdeZ with and without the N-terminal methionine and signal peptide and IdeS/IdeZ hybrid proteins described in international patent application publication WO 2016/128559. Other proteases that may be used in the invention include, for example and without limitation, proteases described in Jordan et al. (2017, N. Engl. J. Med., 377: 442-453), Lannergard and Guss (2006, FEMS Microbiol. Lett., 262:230-235) and Hulting et al., (2009, FEMS Microbiol. Lett., 298:44-50), for example.

Glycosidases are enzymes that hydrolyzes glycosidic bonds in complex sugars. There are generally two broad groups, namely exoglycosidases and endoglycosidases. Glycosidases cleave and thereby releases glycans/oligosaccharides from glycoproteins such as antibodies.

Exoglycosidases that may be used in the invention include, for example and without limitation, N-acetylglucosaminidase, fucosidase, galactosidase, glucosidase, mannosidase, neuraminidase, and xylosidase. Endoglycosidases that may be used in the invention include, for example and without limitation, EndoS, Endo D, endoglycosidase-H, Endo F1, Endo F2 and Endo F3.

Accordingly, in certain embodiments, a glycosidase comprises an endoglycosidase. In certain embodiments, a glycosidase comprises an exoglycosidase.

An example of an endoglycosidase includes, for example and without limitation, EndoS. In certain embodiments, an endoglycosidase comprises a sequence set forth in any of SEQ ID NOs:44-47, or a modified variant thereof.

In certain embodiments, an EndoS polypeptide includes an EndoS polypeptide, a fragment of an EndoS polypeptide, a variant of an EndoS polypeptide, or a variant of a fragment of an EndoS polypeptide, provided that said polypeptide, fragment, variant or variant of fragment has immunoglobulin (Ig) endoglycosidase activity.

In certain embodiments, an EndoS polypeptide is S. pyogenes EndoS. A variant of an EndoS polypeptide may be an EndoS polypeptide from another organism, such as another bacterium. In certain embodiments, a bacterium is a Streptococcus, such as Streptococcus equi, Streptococcus zooepidemicus or Streptococcus pyogenes. Alternatively, the variant may be from Corynebacterium pseudotuberculosis, for example the CP40 protein; Enterococcus faecalis, for example the EndoE protein; or Elizabethkingia meningoseptica (formerly Flavobacterium meningosepticum), for example the EndoF2 protein.

An EndoS polypeptide may comprise or consist of (a) the amino acid sequence of any of SEQ ID NOs:44-47; or (b) a fragment of (a) having Ig endoglycosidase activity; or (c) a variant of (a) having at least 50% identity to the amino acid sequence of any of SEQ ID NOs:44-47 and having Ig endoglycosidase activity; or (d) a variant of (b) having at least 50% identity to the corresponding portion of the amino acid sequence of any of SEQ ID NOs:44-47 and having Ig endoglycosidase activity.

In certain embodiments, the variant polypeptide has at least about 60% or more identity (e.g., 60-70%, 70-80% or 80-90% identity) to the amino acid sequence of any of SEQ ID NOs:44-47, or a fragment thereof having Ig endoglycosidase activity. In certain embodiments, the variant polypeptide has 90-100% identity to the amino acid sequence of any of SEQ ID NOs:44-47, or a fragment thereof having Ig endoglycosidase activity.

A protease may comprise or consist of (a) the amino acid sequence of any of SEQ ID NOs:3-43 or 48; or (b) a fragment of (a) having protease activity; or (c) a variant of (a) having at least 50% identity to the amino acid sequence of any of SEQ ID NOs:3-43 or 48 and having protease activity; or (d) a variant of (b) having at least 50% identity to the corresponding portion of the amino acid sequence of any of SEQ ID NOs:3-43 or 48 and having protease activity. In certain embodiments, the variant polypeptide has at least about 60% or more identity (e.g., 60-70%, 70-80% or 80-90% identity) to the amino acid sequence of any of SEQ ID NOs:3-43 or 48, or a fragment thereof having protease activity. In certain embodiments, the variant polypeptide has 90-100% identity to the amino acid sequence of any of SEQ ID NOs:3-43 or 48, or a fragment thereof having protease activity.

In certain embodiments, the protease or glycosidase is devoid of its native signal sequence and has an additional N-terminal methionine, such as SEQ ID NO:48 which is the mature form of IdeS of S. pyogenes (without the signal sequence, but having an added N-terminal methionine. Any protease or glycosidase used according to methods of the invention can include an added N-terminal methionine in place of the native signal sequence.

A protease or glycosidase can be administered to a subject at any suitable dose. For example, a suitable dosage may be from about 0.05 mg/kg to about 5 mg/kg body weight of a subject, or from about 0.1 mg/kg to about 4 mg/kg body weight of a subject.

In certain embodiments, IdeZ is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

In certain embodiments, IdeS is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

In certain embodiments, EndoS is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

Methods according to the invention can be performed in any suitable order unless otherwise indicated herein. In certain embodiments, a method may comprise first (a) administering to a subject an amount of an agent that reduces interaction of IgG with FcRn effective to reduce neutralizing antibody titers; and then (b) administering to the subject a recombinant viral vector. In certain embodiments, (b) administering to a subject a recombinant viral vector, is performed between about 1 minute to about 90 days after (a) administering to a subject an agent that reduces interaction of IgG with FcRn. In certain embodiments, (a) administering to a subject an agent that reduces interaction of IgG with FcRn, and (b) administering to the subject a recombinant viral vector, are performed at about the same time.

In certain embodiments, a method may comprise first (a) administering to a subject a recombinant viral vector bearing a heterologous polynucleotide and then (b) administering to the subject an amount of an agent that reduces interaction of IgG with FcRn effective to reduce circulating levels of antibodies that bind to a recombinant viral vector and/or heterologous polynucleotide or protein or peptide encoded by the heterologous polynucleotide. In certain embodiments, (b) administering to the subject an amount of an agent that reduces interaction of IgG with FcRn effective to reduce circulating levels of antibodies that bind to a recombinant viral vector and/or heterologous polynucleotide or protein or peptide encoded by the heterologous polynucleotide, is performed between about 1 minute to about 90 days after (a) administering to a subject a recombinant viral vector bearing the heterologous polynucleotide. In certain embodiments, (a) administering to a subject a recombinant viral vector bearing a heterologous polynucleotide, and (b) administering to the subject an amount of an agent that reduces interaction of IgG with FcRn effective to reduce circulating levels of antibodies that bind to a recombinant viral vector and/or heterologous polynucleotide or protein or peptide encoded by the heterologous polynucleotide, are performed at about the same time.

Methods according to the invention may optionally include administering to a subject a protease and/or glycosidase before, after or at about the same time as administration of an agent that reduces interaction of IgG with FcRn or administration of a recombinant viral vector.

In certain embodiments, a method may comprise first (a) administering to a subject an agent that reduces interaction of IgG with FcRn; and then (b) administering to the subject a protease and/or glycosidase effective to degrade or digest neutralizing antibodies; and then (c) administering to the subject a recombinant viral vector.

In certain embodiments, a method may comprise first (a) administering to a subject an agent that reduces interaction of IgG with FcRn; and then (b) administering to the subject an endopeptidase effective to degrade or digest neutralizing antibodies; and then (c) administering to the subject a recombinant viral vector.

In certain embodiments, a method may comprise first (a) administering to a subject an anti-FcRn antibody; and then (b) administering to the subject an IdeS effective to degrade or digest neutralizing antibodies; and then (c) administering to the subject a recombinant viral vector.

Antibodies, such as neutralizing antibodies, may be preexisting and may be present in a subject, even before administration of a viral vector, at levels that inhibit or reduce recombinant viral vector cell transduction. Alternatively, antibodies may develop in a subject after exposure to a virus upon which the recombinant viral vector is based. Still further, antibodies, such as neutralizing antibodies, or antibodies that bind to the heterologous polynucleotide or a protein or peptide encoded by the heterologous polynucleotide encapsidated by the viral vector may develop in a subject after administration of a recombinant viral vector.

Accordingly, the methods herein are applicable to subjects with pre-existing antibodies and subjects without pre-existing antibodies. As set forth herein, such subjects include subjects that have been exposed to wild-type virus and develop pre-existing antibodies against the viral vector based upon the wild-type virus, as well as subjects that have received a viral vector gene therapy treatment and have developed antibodies and may be subsequently treated with one or more additional doses of the same viral vector gene therapy (referred to as redosing) or be treated with a different gene therapy treatment (e.g., a different heterologous polynucleotide) using the same viral vector to deliver the gene therapy treatment.

A subject may be tested for antibodies prior to viral vector administration and/or prior to administration of an agent that reduces interaction of IgG with FcRn, a protease or a glycosidase. Nonlimiting examples of antibodies to test for include neutralizing antibodies, antibodies that bind to an agent that reduces interaction of IgG with FcRn or a protease or glycosidase as set forth herein, antibodies that bind to the heterologous polynucleotide and antibodies that bind to the protein or peptide encoded by the heterologous polynucleotide. Subjects can therefore be screened for neutralizing antibodies, antibodies that bind to an agent that reduces interaction of IgG with FcRn or a protease or glycosidase as set forth herein, antibodies that bind to a heterologous polynucleotide or antibodies that bind to a protein or peptide encoded by the heterologous polynucleotide, prior to administration of a recombinant viral vector and/or prior to administration of an agent that reduces interaction of IgG with FcRn or a protease or glycosidase.

Subjects that have pre-existing antibodies (e.g., IgG) that bind to an agent that reduces interaction of IgG with FcRn or a protease or glycosidase as set forth herein, can optionally be excluded from initial treatment by a method according to the invention method. However, not all subjects that have or develop antibodies that bind to an agent that reduces interaction of IgG with FcRn or a protease or glycosidase need be excluded from treatment methods according to the invention. For example, a subject with detectable anti-IdeS IgG having a titer of less than 15 mg/per liter can still be treated using methods according to the invention. It is expected that reduction of circulating IgG by administration of an agent that reduces interaction of IgG with FcRn will also result in reduction of antibodies that may bind the agent that reduces interaction of IgG with FcRn or IdeS.

Subjects also can be screened for neutralizing antibodies, antibodies that bind to a heterologous polynucleotide or antibodies that bind to a protein or peptide encoded by the heterologous polynucleotide after administration of a recombinant viral vector. Such subjects can optionally be monitored for a period of time after administration of the recombinant viral vector in order to determine if such antibodies develop or are prevented from developing in a subject in which pre-existing antibodies have not been detected, or in the case of a subject with pre-existing antibodies whether an agent that reduces interaction of IgG with FcRn and a protease and/or glycosidase decreases or eliminates such pre-existing antibodies.

In certain embodiments, an agent that reduces interaction of IgG with FcRn is administered to a subject after testing positive for the presence of neutralizing antibodies, antibodies that bind to an agent that reduces interaction of IgG with FcRn as set forth herein, antibodies that bind to a heterologous polynucleotide or antibodies that bind to a protein or peptide encoded by the heterologous polynucleotide. In certain embodiments, an agent that reduces interaction of IgG with FcRn is administered to a subject before testing positive for the presence of neutralizing antibodies, antibodies that bind to an agent that reduces interaction of IgG with FcRn as set forth herein, antibodies that bind to a heterologous polynucleotide or antibodies that bind to a protein or peptide encoded by the heterologous polynucleotide.

In certain embodiments, an agent that reduces interaction of IgG with FcRn and a protease and/or glycosidase is administered to a subject after testing positive for the presence of neutralizing antibodies, antibodies that bind to an agent that reduces interaction of IgG with FcRn or a protease or glycosidase as set forth herein, antibodies that bind to a heterologous polynucleotide or antibodies that bind to a protein or peptide encoded by the heterologous polynucleotide. In certain embodiments, an agent that reduces interaction of IgG with FcRn and a protease and/or glycosidase is administered to a subject before testing positive for the presence of neutralizing antibodies, antibodies that bind to an agent that reduces interaction of IgG with FcRn, a protease or glycosidase as set forth herein, antibodies that bind to a heterologous polynucleotide or antibodies that bind to a protein or peptide encoded by the heterologous polynucleotide.

In certain embodiments, subjects are not tested for antibodies prior to or after administration of an agent that reduces interaction of IgG with FcRn, or prior to or after administration of an agent that reduces interaction of IgG with FcRn and a protease and/or glycosidase. Accordingly, testing for neutralizing antibodies, antibodies that bind to an agent that reduces interaction of IgG with FcRn or a protease or glycosidase as set forth herein, antibodies that bind to a heterologous polynucleotide or antibodies that bind to a protein or peptide encoded by the heterologous polynucleotide after administration of a protease and/or glycosidase or administration of a recombinant viral vector is optional in treatment methods according to the invention.

An agent that reduces interaction of IgG with FcRn can be administered to a subject any number of times. For example, an agent that reduces interaction of IgG with FcRn can be administered 2 to 5 times, 2 to 10 times, 2 to 15 times to a subject.

An agent that reduces interaction of IgG with FcRn can be administered to a subject for any duration of time on a regular basis, such as consecutive days, or alternating days, or an irregular basis. In certain embodiments, an agent that reduces interaction of IgG with FcRn is administered from about 1 to 12 weeks, or from about 1 to 10 weeks, or from about 1 to 8 weeks, or from about 1 to 6 weeks, or from about 1 to 4 weeks, or from about 1 to 3 weeks, or from about 1 to 2 weeks, or about 2 weeks before or after administration of a recombinant viral vector.

A protease or glycosidase can be administered to a subject any number of times. For example, a protease and/or glycosidase can be administered 2 to 5 times, 2 to 10 times, 2 to 15 times to a subject.

A protease or glycosidase can be administered to a subject for any duration of time on a regular basis, such as consecutive days, or alternating days, or an irregular basis. In certain embodiments, a protease and/or glycosidase is administered from about 1 to 12 weeks, or from about 1 to 10 weeks, or from about 1 to 8 weeks, or from about 1 to 6 weeks, or from about 1 to 4 weeks, or from about 1 to 3 weeks, or from about 1 to 2 weeks, or about 2 weeks before or after administration of an agent that reduces interaction of IgG with FcRn or administration of a recombinant viral vector.

In certain embodiments, a recombinant viral vector is administered before or after an agent that reduces interaction of IgG with FcRn, or before or after a protease or glycosidase is administered to a subject. In certain embodiments, a recombinant viral vector is administered to a subject e.g., 1-12, 12-24 or 24-48 hours, or 2-4, 4-6, 6-8, 8-10, 10-14, 14-20, 20-25, 25-30, 30-50, or more than 50 days following administering an agent that reduces interaction of IgG with FcRn or a protease or glycosidase to the subject. In certain embodiments, an agent that reduces interaction of IgG with FcRn or a protease or glycosidase is administered to a subject e.g., 1-12, 12-24 or 24-48 hours, or 2-4, 4-6, 6-8, 8-10, 10-14, 14-20, 20-25, 25-30, 30-50, or more than 50 days following administering a recombinant viral vector to the subject.

The recombinant viral vector, the agent that reduces interaction of IgG with FcRn, protease and/or glycosidase may be administered alone or in a combination. In certain embodiments, a recombinant viral vector is administered to a subject separately from an agent that reduces interaction of IgG with FcRn and/or a protease and/or glycosidase. In certain embodiments, a recombinant viral vector is administered to a subject in combination with an agent that reduces interaction of IgG with FcRn and/or a protease and/or glycosidase.

In certain embodiments, a mixture of an agent that reduces interaction of IgG with FcRn and a protease and/or glycosidase is administered to a subject, one or more times. In certain embodiments, two or more agents that reduce interaction of IgG with FcRn or two or more proteases and/or glycosidases are administered to a subject, one or more times.

In certain embodiments, at least one immunosuppressive agent is administered to a subject prior to, substantially contemporaneously with or after administration of a recombinant viral vector, agent that reduces interaction of IgG with FcRn, protease or glycosidase to the subject. In certain embodiments, an immunosuppressive agent is an anti-inflammatory agent such as a steroid. In certain embodiments, an immunosuppressive agent is prednisone, cyclosporine (e.g., cyclosporine A), mycophenolate, rituximab, rapamycin or a derivative thereof.

Additional strategies to reduce humoral immunity include methods to remove, deplete, capture, and/or inactivate antibodies, commonly referred to as apheresis and more particularly, plasmapheresis where blood products are involved. Apheresis or plasmapheresis, is a process in which a human subject's plasma is circulated ex vivo (extracorporal) through a device that modifies the plasma through addition, removal and/or replacement of components before its return to the patient. Plasmapheresis can be used to remove human immunoglobulins (e.g., IgG, IgE, IgA, IgD) from a blood product (e.g., plasma). This procedure depletes, captures, inactivates, reduces or removes immunoglobulins (antibodies) that bind a recombinant viral vector, bind to a heterologous polynucleotide, bind to a protein or peptide encoded by the heterologous polynucleotide, bind to an agent that reduces interaction of IgG with FcRn, bind to a protease and/or bind to a glycosidase thereby reducing the titer of antibodies in the treated subject that may contribute, for example, to viral vector neutralization. An example is a device composed of an AAV capsid affinity matrix column. Passing blood product (e.g., plasma) through such an AAV capsid affinity matrix would result in binding only of AAV antibodies, and of all isotypes (including IgG, IgM, etc.). Immunoadsorption (US Patent Application publication US 2018/0169273 A1) can also be used to deplete immunoglobulins, and more particularly anti-AAV antibodies. Affinity ligands (international patent application publication WO/2018/158397) can also be used to deplete immunoglobulins, and more particularly anti-AAV antibodies. Any of the aforementioned strategies can be used prior to, substantially contemporaneously with or after administration of a recombinant viral vector, agent that reduces interaction of IgG with FcRn, protease or glycosidase to the subject.

In certain embodiments, plasmapheresis is performed on a blood product (e.g., plasma) of a subject as an additional one or more step in a method of the invention, before or after administration to the subject of an agent that reduces interaction of IgG with FcRn and/or, before or after administration of a recombinant viral vector to the subject.

In certain embodiments, plasmapheresis is performed on a blood product (e.g., plasma) of a subject before or after administration of an agent that reduces interaction of IgG with FcRn, and then a recombinant viral vector is administered to the subject.

In certain embodiments, plasmapheresis is performed on a blood product (e.g., plasma) of a subject before or after administration of an agent that reduces interaction of IgG with FcRn, then a protease or glycosidase is administered to the subject, and then a recombinant viral vector is administered to the subject.

In certain embodiments, plasmapheresis is performed on a blood product (e.g., plasma) of a subject after administration of an agent that reduces interaction of IgG with FcRn, then IdeS or EndoS is administered to the subject, and then a recombinant viral vector is administered to the subject.

In certain embodiments, plasmapheresis is performed on a blood product (e.g., plasma) of a subject after administration of an agent that reduces interaction of IgG with FcRn, then IdeS is administered to the subject, and then a recombinant viral vector is administered to the subject.

Use of plasmapheresis in addition to administration of an agent that reduces interaction of IgG with FcRn and administration of a protease (e.g., IdeS) or glycosidase (e.g., EndoS) in methods of the invention may be particularly beneficial to gene therapy redosing treatment where may be a high titer of circulating neutralizing antibodies in the subject.

Additional strategies to reduce (overcome) or avoid humoral immunity to AAV in systemic gene transfer include use of AAV empty capsid particles and/or capsid proteins as decoys to adsorb anti-AAV antibodies, administration of immunosuppressive drugs to decrease, reduce, inhibit, prevent or eradicate the humoral immune response to AAV, changing the AAV capsid serotype or engineering the AAV capsid to be less susceptible to neutralizing antibodies (NAb), use of plasma exchange cycles to adsorb anti-AAV immunoglobulins, thereby reducing anti-AAV antibody titer, and use of delivery techniques such as balloon catheters followed by saline flushing. Still further strategies are described in Mingozzi et al., 2013, Blood, 122:23-36. Additional strategies include use of AAV-specific plasmapheresis columns to selectively deplete anti-AAV antibodies without depleting the total immunoglobulin pool from plasma, as described in Bertin et al., 2020, Sci. Rep. 10:864. Apheresis strategies to remove, deplete, capture, and/or inactivate AAV antibodies in subjects are described in WO2019018439.

In accordance with the invention, an agent that reduces interaction of IgG with FcRn may be encapsulated or complexed with liposomes, nanoparticles, lipid nanoparticles, polymers, microparticles, microcapsules, micelles, or extracellular vesicles.

Also in accordance with the invention, viral particles may be encapsulated or complexed with liposomes, nanoparticles, lipid nanoparticles, polymers, microparticles, microcapsules, micelles, or extracellular vesicles.

Also in accordance with the invention, a protease and/or glycosidase may be encapsulated or complexed with liposomes, nanoparticles, lipid nanoparticles, polymers, microparticles, microcapsules, micelles, or extracellular vesicles.

A “lipid nanoparticle” or “LNP” refers to a lipid-based vesicle useful for delivery of recombinant viral vector and or protease and/or glycosidase and having dimensions on the nanoscale, i.e., from about 10 nm to about 1000 nm, or from about 50 to about 500 nm, or from about 75 to about 127 nm. Without being bound by theory, LNP is believed to provide the protease, glycosidase, or recombinant viral vector with partial or complete shielding from the immune system. Shielding allows delivery of the protease, glycosidase, or recombinant viral vector to a tissue or cell while avoiding inducing a substantial immune response against the protease, glycosidase, or recombinant viral vector in vivo. Shielding may also allow repeated administration without inducing a substantial immune response against the protease, glycosidase, or recombinant viral vector in vivo (e.g., in a subject such as a human). Shielding may also improve or increase delivery efficiency, duration of therapeutic effect and/or therapeutic efficacy in vivo.

The pI (isoelectric point) of AAV is in a range from about 6 to about 6.5. Thus, the AAV surface carries a slight negative charge. As such it may be beneficial for the LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids may also be titratable cationic lipids. In certain embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

Cationic lipids may include, without limitation, 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include, but are not limited to, 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, γ-DLen-C2K-DMA, and (DLin-MP-DMA) (also known as 1-B11).

Still further cationic lipids may include, without limitation, 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

In certain embodiments, cationic lipid may be present in an amount from about 10% by weight of the LNP to about 85% by weight of the lipid nanoparticle, or from about 50% by weight of the LNP to about 75% by weight of the LNP.

Sterols may confer fluidity to the LNP. As used herein, “sterol” refers to any naturally occurring sterol of plant (phytosterols) or animal (zoosterols) origin as well as non-naturally occurring synthetic sterols, all of which are characterized by the presence of a hydroxyl group at the 3-position of the steroid A-ring. The sterol can be any sterol conventionally used in the field of liposome, lipid vesicle or lipid particle preparation, most commonly cholesterol. Phytosterols may include campesterol, sitosterol, and stigmasterol. Sterols also includes sterol-modified lipids, such as those described in U.S. Patent Application Publication 2011/0177156. In certain embodiments, a sterol may be present in an amount from about 5% by weight of the LNP to about 50% by weight of the lipid nanoparticle or from about 10% by weight of the LNP to about 25% by weight of the LNP.

LNP can comprise a neutral lipid. Neutral lipids may comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In certain embodiments, the neutral lipid component may be a lipid having two acyl groups (e.g., diacylphosphatidylcholine and diacylphosphatidylethanolamine).

Lipids having a variety of acyl chain groups of varying chain length and degree of saturation are available or may be isolated or synthesized by well-known techniques. In certain embodiments, lipids containing saturated fatty acids with carbon chain lengths in the range of C14 to C22 may be used. In certain embodiments, lipids with mono or diunsaturated fatty acids with carbon chain lengths in the range of C14 to C22 are used. Additionally, lipids having mixtures of saturated and unsaturated fatty acid chains can be used. Exemplary neutral lipids include, without limitation, 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or any related phosphatidylcholine. The neutral lipids may also be composed of sphingomyelin, dihydrosphingomyelin, or phospholipids with other head groups, such as serine and inositol.

In certain embodiments, the neutral lipid may be present in an amount from about 0.1% by weight of the lipid nanoparticle to about 75% by weight of the LNP, or from about 5% by weight of the LNP to about 15% by weight of the LNP.

LNP encapsulated protease, glycosidase, or recombinant viral vector can be incorporated into pharmaceutical compositions, e.g., a pharmaceutically acceptable carrier or excipient. Such pharmaceutical compositions are useful for, among other things, administration and delivery of LNP encapsulated protease, glycosidase, or recombinant viral vector to a subject in vivo or ex vivo.

Preparations of LNP can be combined with additional components, which may include, for example and without limitation, polyethylene glycol (PEG) and sterols.

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments, PEG may be a polyethylene glycol with an average molecular weight of about 550 to about 10,000 daltons and is optionally substituted by alkyl, alkoxy, acyl or aryl. In certain embodiments, the PEG may be substituted with methyl at the terminal hydroxyl position. In certain embodiments, the PEG may have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In certain embodiments, the terminal hydroxyl group may be substituted with a methoxy or methyl group.

PEG-modified lipids include the PEG-dialkyloxypropyl conjugates (PEG-DAA) described in U.S. Pat. Nos. 8,936,942 and 7,803,397. PEG-modified lipids (or lipid-polyoxyethylene conjugates) that are useful may have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, PEG-modified dialkylamines and PEG-modified 1,2-diacyloxypropan-3-amines. In certain embodiments, the PEG-modified lipid may be PEG-modified diacylglycerols and dialkylglycerols. In certain embodiments, the PEG may be in an amount from about 0.5% by weight of the LNP to about 20% by weight of the LNP, or from about 5% by weight of the LNP to about 15% by weight of the LNP.

Furthermore, LNP can be a PEG-modified and a sterol-modified LNP. The LNPs, combined with additional components, can be the same or separate LNPs. In other words, the same LNP can be PEG modified and sterol modified or, alternatively, a first LNP can be PEG modified and a second LNP can be sterol modified. Optionally, the first and second modified LNPs can be combined.

In certain embodiments, prior to encapsulating LNPs may have a size in a range from about 10 nm to 500 nm, or from about 50 nm to about 200 nm, or from 75 nm to about 125 nm. In certain embodiments, LNP encapsulated protease, glycosidase, or recombinant viral vector may have a size in a range from about 10 nm to 500 nm.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA).

Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” polynucleotide or nucleic acid sequence refers to a polynucleotide inserted into a plasmid or vector for purposes of vector mediated transfer/delivery of the polynucleotide into a cell. Heterologous nucleic acid sequences are distinct from viral nucleic acid, i.e., are non-native with respect to viral nucleic acid. Once transferred/delivered into the cell, a heterologous nucleic acid sequence, contained within the vector, can be expressed (e.g., transcribed, and translated if appropriate). Alternatively, a transferred/delivered heterologous polynucleotide in a cell, contained within the vector, need not be expressed. Although the term “heterologous” is not always used herein in reference to nucleic acid sequences and polynucleotides, reference to a nucleic acid sequence or polynucleotide even in the absence of the modifier “heterologous” is intended to include heterologous nucleic acid sequences and polynucleotides in spite of the omission.

A “transgene” is used herein to conveniently refer to a nucleic acid that is intended or has been introduced into a cell or organism. Transgenes include any nucleic acid, such as a heterologous polynucleotide sequence or a heterologous nucleic acid encoding a protein or peptide. The term transgene and heterologous nucleic acid/polynucleotide sequences are used interchangeably herein.

In certain embodiments, a heterologous polynucleotide encodes a protein selected from the group consisting of GAA (acid alpha-glucosidase) for treatment of Pompe disease; ATP7B (copper transporting ATPase2) for treatment of Wilson's disease; alpha galactosidase for treatment of Fabry's disease; ASS1 (arginosuccinate synthase) for treatment of Citrullinemia Type 1; beta-glucocerebrosidase for treatment of Gaucher disease Type 1; beta-hexosaminidase A for treatment of Tay Sachs disease; SERPING1 (C1 protease inhibitor or C1 esterase inhibitor) for treatment of hereditary angioedema (HAE), also known as C1 inhibitor deficiency type I and type II; and glucose-6-phosphatase for treatment of glycogen storage disease type I (GSDI).

In certain embodiments, a heterologous polynucleotide encodes a protein selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In certain embodiments, a heterologous polynucleotide encodes acid α-glucosidase (GAA). Administration of a recombinant viral vector comprising a heterologous polynucleotide encoding GAA to a subject with Pompe or another glycogen storage disease can lead to the expression of the GAA protein. Expression of GAA protein in the patient may serve to suppress, inhibit or reduce the accumulation of glycogen, prevent the accumulation of glycogen or degrade glycogen, which in turn can reduce or decrease one or more adverse effects of Pompe disease, or another glycogen storage disease.

In certain embodiments, a heterologous polynucleotide encodes a protein selected from the group consisting of thrombopoietin (TPO), an interleukin (IL-1 through IL-36, etc.), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In certain embodiments, a heterologous polynucleotide encodes CFTR (cystic fibrosis transmembrane regulator protein), a blood coagulation (clotting) factor (Factor XIII, Factor IX, Factor VIII, Factor X, Factor VII, Factor VIIa, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, a sulfatase, N-acetylglucosamine-1-phosphate transferase, cathepsin A, GM2-AP, NPC1, VPC2, a sphingolipid activator protein, one or more zinc finger nucleases for genome editing, or one or more donor sequences used as repair templates for genome editing.

In certain embodiments, a heterologous polynucleotide encodes erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C-X-C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases; keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alpha1-antitrypsin for treatment of emphysema or chronic obstructive pulmonary disease (COPD); alpha-L-iduronidase for treatment of mucopolysaccharidosis I (MPS I); ornithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); ATP-binding cassette, sub-family A (ABC1), member 4 (ABCA4) for the treatment of Stargardt disease; or dystrophin.

The terms “polypeptides,” “proteins” and “peptides” are used interchangeably herein. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of functionality of the native full-length protein. In the invention, such polypeptides, proteins and peptides encoded by the polynucleotide sequences can be but are not required to be identical to the endogenous protein that is defective, or whose expression is insufficient, or deficient in the treated mammal.

In certain embodiments, the heterologous polynucleotide encodes an inhibitory nucleic acid selected from the group consisting of a siRNA, an antisense molecule, miRNA, RNAi, a ribozyme and a shRNA.

In certain embodiments, an inhibitory nucleic acid binds to a gene, a transcript of a gene, or a transcript of a gene associated with a polynucleotide repeat disease selected from the group consisting of a huntingtin (HTT) gene, a gene associated with dentatorubropallidoluysian atrophy (atrophin 1, ATN1), androgen receptor on the X chromosome in spinobulbar muscular atrophy, human Ataxin-1, -2, -3, and -7, Cav2.1 P/Q voltage-dependent calcium channel (CACNA1A), TATA-binding protein, Ataxin 8 opposite strand (ATXN8OS), Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DNA damage-inducible transcript 4 protein, in diabetic macular edema (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; and mutant rhodopsin gene (RHO) in autosomal dominantly inherited retinitis pigmentosa (adRP).

Recombinant viral vector doses can be administered at any appropriate dose. Generally, doses will range from at least 1×10⁸, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. AAV dose in the range of 1×10¹⁰-1×10¹¹ vg/kg in mice, and 1×10¹²-1×10¹³ vg/kg in dogs have been effective. More particularly, a dose from about 1×10¹¹ vg/kg to about 5×10¹⁴ vg/kg inclusive, or from about 5×10¹¹ vg/kg to about 1×10¹⁴ vg/kg inclusive, or from about 5×10¹¹ vg/kg to about 5×10¹³ vg/kg inclusive, or from about 5×10¹¹ vg/kg to about 1×10¹³ vg/kg inclusive, or from about 5×10¹¹ vg/kg or about 5×10¹² vg/kg inclusive, or from about 5×10¹¹ vg/kg to about 1×10¹² vg/kg inclusive. Doses can be, for example, about 5×10¹⁴ vg/kg, or less than about 5×10¹⁴ vg/kg, such as a dose from about 2×10¹¹ to about 2×10¹⁴ vg/kg inclusive, in particular, for example, about 2×10¹² vg/kg, about 6×10¹² vg/kg, or about 2×10¹³ vg/kg.

In certain embodiments, administration to a subject of an agent that reduces the interaction of IgG with FcRn reduces the dose of a recombinant viral vector comprising a therapeutic heterologous polynucleotide required to be effective for gene therapy treatment of a subject. In certain embodiments, administration to a subject of an agent that reduces the interaction of IgG with FcRn allows for administration of an increased dose of a recombinant viral vector comprising a therapeutic heterologous polynucleotide.

In certain embodiments, administration of a protease and/or glycosidase to a subject, in addition to administration to the subject of an agent that reduces the interaction of IgG with FcRn, reduces the dose of a recombinant viral vector comprising a therapeutic heterologous polynucleotide required to be effective for treatment of a subject. In certain embodiments, administration of a protease and/or glycosidase to a subject, in addition to administration to the subject of an agent that reduces the interaction of IgG with FcRn, allows for administration of an increased dose of a recombinant viral vector comprising a therapeutic heterologous polynucleotide.

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the recombinant viral vector, a host immune response to the heterologous polynucleotide or expression product (protein or peptide or transcribed nucleic acid), and the stability of the protein or peptide expressed or nucleic acid transcribed. One skilled in the art can determine a recombinant viral vector genome dose range to treat a patient having a particular disease or disorder based on the aforementioned factors, as well as other factors.

An “effective amount” or “sufficient amount” refers to an amount that provides, in single or multiple doses, alone or in combination, with one or more other compositions, treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured). The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of recombinant GAA for treatment of a lysosomal storage disease (e.g., Pompe disease), or administration of a recombinant clotting factor protein (e.g., FVIII or FIX) for treatment of a clotting disorder (e.g., hemophilia A (HemA) or hemophilia B (HemB)).

For Pompe disease, an effective amount would be an amount of GAA that inhibits or reduces glycogen production or accumulation, enhances or increases glycogen degradation or removal, reduces lysosomal alterations in tissues of the body of a subject, or improves muscle tone and/or muscle strength and/or respiratory function in a subject, for example. Effective amounts can be determined, for example, by ascertaining the kinetics of GAA uptake by myoblasts from plasma. Myoblasts GAA uptake rates (K uptake) of about 141-147 nM may appear to be effective (see, e.g., Maga et al., J. Biol. Chem. 2012) In animal models, GAA activity levels in plasma of greater than about 1,000 nmol/hr/mL, for example, about 1,000 to about 2,000 nmol/hr/mL have been observed to be therapeutically effective.

For HemA and HemB, generally speaking, it is believed that, in order to achieve a therapeutic effect, a blood coagulation factor concentration that is greater than 1% of factor concentration found in a normal individual is needed to change a severe disease phenotype to a moderate one. A severe phenotype is characterized by joint damage and life-threatening bleeds. To convert a moderate disease phenotype into a mild one, it is believed that a blood coagulation factor concentration greater than 5% of normal is needed.

FVIII and FIX levels in normal humans are about 150-200 ng/mL plasma, but may be less (e.g., range of about 100-150 ng/mL) or greater (e.g., range of about 200-300 ng/mL) and still considered normal, due to functional clotting as determined, for example, by an activated partial thromboplastin time (aPTT) one-stage clotting assay. Thus, a therapeutic effect can be achieved such that the total amount of FVIII or FIX in the subject/human is greater than 1% of the FVIII or FIX present in normal subjects/humans, e.g., 1% of 100-300 ng/mL.

The composition can be administered to a subject as a combination composition, or administered separately, such as concurrently or in series or sequentially (prior to or following) delivery or administration of a recombinant viral vector comprising a heterologous polynucleotide. The invention provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a recombinant viral vector comprising a heterologous polynucleotide, to a subject.

Accordingly, the invention includes, inter alia, methods and uses that result in a reduced need or use of another compound, agent, drug, therapeutic regimen, treatment protocol, process, or remedy. For example, for a blood clotting disease, a method of treatment according to the invention has a therapeutic benefit if in a given subject a less frequent or reduced dose or elimination of administration of a recombinant clotting factor protein to supplement for the deficient or defective (abnormal or mutant) endogenous clotting factor in the subject. In another example, for a lysosomal storage disease, such as Pompe disease, a method of treatment according to the invention has a therapeutic benefit even if a less frequent or reduced dose of a recombinant viral vector comprising GAA has been previously administered, or continues to be administered to a subject. Thus, reducing the need for, or the use of, another treatment or therapy is included in the invention.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease. For Pompe, an effective amount would be an amount that inhibits or reduces glycogen production or accumulation, enhances or increases glycogen degradation or removal, improves muscle tone and/or muscle strength and/or respiratory function, for example. For HemA or HemB, an effective amount would be an amount that reduces frequency or severity of acute bleeding episodes in a subject, for example, or an amount that reduces clotting time as measured by a clotting assay, for example.

Accordingly, pharmaceutical compositions of the invention include compositions wherein the active ingredients are contained in an effective amount to achieve the intended therapeutic purpose. Determining a therapeutically effective dose is well within the capability of a skilled medical practitioner using techniques and guidance known in the art and using the teachings provided herein.

Therapeutic doses will depend on, among other factors, the age and general condition of the subject, the severity of the aberrant phenotype, and the strength of the control sequences regulating expression levels. Thus, a therapeutically effective amount in humans will fall in a relatively broad range that may be determined by a medical practitioner based on the response of an individual patient to a vector-based treatment. Such doses may be alone or in combination with an immunosuppressive agent or drug.

Compositions such as pharmaceutical compositions may be delivered to a subject, so as to allow transgene expression and optionally production of encoded protein. In certain embodiments, pharmaceutical compositions comprising sufficient genetic material to enable a subject to produce a therapeutically effective amount of a blood-clotting factor to improve hemostasis in the subject. In certain embodiments, pharmaceutical compositions comprising sufficient heterologous polynucleotide to enable a subject to produce a therapeutically effective amount of GAA.

In certain embodiments, a therapeutic effect in a subject is sustained for a period of time, e.g., 2-4, 4-6, 6-8, 8-10, 10-14, 14-20, 20-25, 25-30, or 30-50 days or more, for example, 50-75, 75-100, 100-150, 150-200 days or more. Accordingly, in certain embodiments, a recombinant viral vector provides a therapeutic effect.

In certain embodiments, a recombinant viral vector provides a therapeutic effect without an immunosuppressive agent. In certain embodiments, at least one immunosuppressive agent is administered to a subject prior to, substantially contemporaneously with or after administration of a recombinant viral vector to the subject.

In certain embodiments, an immunosuppressive agent is an anti-inflammatory agent. In certain embodiments, an immunosuppressive agent is a steroid. In certain embodiments, an immunosuppressive agent is prednisone, prednisolone, calcineurin inhibitor, cyclosporine (e.g., cyclosporine A), tacrolimus, mycophenolate, CD52 inhibitor (e.g., alemtuzumab), CTLA4-Ig (e.g., abatacept, belatacept), anti-CD3 mAb, anti-LFA-1 mAb (e.g., efalizumab), anti-CD40 mAb (e.g., ASKP1240), anti-CD22 mAb (e.g., epratuzumab), anti-CD20 mAb (e.g., rituximab, orelizumab, ofatumumab, veltuzumab), rapamycin or a derivative thereof. Additional particular agents include a stabilizing compound. Other immunosuppressive agents that can be used in methods according to the invention include, for example and without limitation, TACI-Ig (e.g., atacicept), anti-C5 mAb (e.g., eculizumab), mycophenolate, azathioprine, sirolimus, everolimus, TNFR-Ig (e.g., etanercept (Enbrel®), anti-TNF mAb (e.g., adalimumab (Humira @), infliximab (Remicade®; Avsola®)), tofacitinib, anti-IL-2R (e.g., basiliximab), anti-IL-17 mAb (e.g., secukinumab), anti-IL-6 mAb (e.g., anti-IL-6 antibody sirukumab, anti-IL-6 receptor antibody tocilizumab (Actemra®), IL-10 inhibitor, TGF-beta inhibitor, a B cell targeting antibody (e.g., rituximab), a proteasome inhibitor (e.g., bortezomib), a mammalian target of rapamycin (mTOR) inhibitor (e.g., rapamycin), synthetic vaccine particle (SVP™)-rapamycin (rapamycin encapsulated in a biodegradable nanoparticle), intravenous gamma globulin (IVIG), omalizumab, methotrexate, a tyrosine kinase inhibitor (e.g., ibrutinib), an inhibitor of B-cell activating factor (BAFF) (e.g., anti-BAFF mAb, e.g., belimumab), an inhibitor of a proliferation-inducing ligand (APRIL), anti-IL-1b mAb (e.g., canakinumab (Haris®)), a C3a inhibitor, a Tregitope (see, e.g., U.S. Pat. No. 10,213,496), or a combination and/or derivative thereof.

Compositions may be administered in any sterile, biocompatible pharmaceutical carrier, including, but not limited to, saline, buffered saline, dextrose, and water. The compositions may be administered to a patient alone, or in combination with other agents, which influence dosage amount, administration frequency and/or therapeutic efficacy.

Methods and uses of the invention include delivery and administration systemically, regionally or locally, or by any route, for example, by injection or infusion. Delivery of the pharmaceutical compositions in vivo may generally be accomplished via injection using a conventional syringe, although other delivery methods such as convection-enhanced delivery are envisioned (See e.g., U.S. Pat. No. 5,720,720). For example, compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in the treatment of patients with blood coagulation disorders may determine the optimal route for administration of the adenoviral-associated vectors based on a number of criteria, including, but not limited to, the condition of the patient and the purpose of the treatment (e.g., increased GAA, enhanced blood coagulation, etc.).

Methods of treatment according to the invention include combination therapies that include the additional use of any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs. Such biologics (proteins), agents, drugs, treatments and therapies can be administered or performed prior to, substantially contemporaneously with or following any other method of treatment according to the invention, for example, a therapeutic method of treating a subject for a lysosomal storage disease such as Pompe, or a therapeutic method of treating a subject for a blood clotting disease such as HemA or HemB.

The compound, agent, drug, treatment or other therapeutic regimen or protocol can be administered as a combination composition, or administered separately, such as concurrently or in series or sequentially (prior to or following) delivery or administration of a nucleic acid, vector, recombinant vector (e.g., recombinant viral vector), or recombinant virus particle. The invention therefore provides combinations in which a method of treatment according to the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, vector, recombinant vector (e.g., recombinant viral vector), or recombinant virus particle administered to a patient or subject according to the invention.

In certain embodiments, administration of an agent that reduces interaction of IgG with FcRn to a subject may lead to prevention of development of neutralizing antibodies, antibodies that bind to the heterologous polynucleotide and/or antibodies that bind to a protein or peptide encoded by the heterologous polynucleotide. As set forth herein, administration of the agent that reduces interaction of IgG with FcRn to such a subject can be prior to administration of a viral vector, substantially contemporaneously at the time of administration of a viral vector, or after administration of a viral vector to the subject.

In certain embodiments, administration of agent that reduces interaction of IgG with FcRn to a subject with pre-existing antibodies leads to reduction of neutralizing antibodies, antibodies that bind to the heterologous polynucleotide and/or antibodies that bind to a protein or peptide encoded by the heterologous polynucleotide. After administration of the agent that reduces interaction of IgG with FcRn, such subjects can then be administered a recombinant viral vector in accordance with the methods herein. Such subjects may optionally be evaluated for presence of remaining pre-existing antibodies after administration of a recombinant viral vector. Alternatively, such subjects can be administered the recombinant viral vector after a predetermined amount of time has passed during which the agent that reduces interaction of IgG with FcRn reduces or eliminates any such pre-existing antibodies in the subject.

In certain embodiments, administration of agent that reduces interaction of IgG with FcRn to a subject may lead to reduction, degradation or digestion of at least 20% to 50%, at least 55%, at least 60%, at least 65%, at least 70%, at least 75%, at least 80%, at least 85%, at least 90%, at least 95%, at least 98%, at least 99%, or 100% of neutralizing antibodies, antibodies that bind to the heterologous polynucleotide and/or antibodies that bind to a protein or peptide encoded by the heterologous polynucleotide, as reflected by measurement of such antibodies in a biological sample obtained from a subject administered a recombinant viral vector. In certain embodiments, a method according to the invention reduces, degrades or digests at least 80%, at least 85%, at least 90%, at least 95%, at least 98%, at least 99%, or 100% of the neutralizing antibodies, and/or antibodies that bind to the heterologous polynucleotide and/or antibodies that bind to a protein or peptide encoded by the heterologous polynucleotide.

Non-limiting examples of a biological sample from a subject that may be analyzed include whole blood, serum, plasma, the like, and a combination thereof. A biological sample may be devoid of cells, or may include cells (e.g., red blood cells, platelets and/or lymphocytes).

In certain embodiments, neutralizing antibodies present in a biological sample of a subject may be reduced, degraded or digested to less than about 1:25, where 1 part of the biological sample diluted in 25 of buffer results in 50% recombinant viral vector neutralization. In certain embodiments, neutralizing antibodies present in a biological sample of the subject may be reduced, degraded or digested to less than about 1:20, less than about 1:15, less than about 1:10, less than about 1:5, less than about 1:4, less than about 1:3, less than about 1:2, or less than about 1:1, where 1 part of the biological sample diluted in 20, 15, 10, 5, 4, 3, 2, or 1 part, respectively, of buffer results in 50% recombinant viral vector neutralization.

Exemplary analysis and measurement of AAV neutralizing antibodies in a biological sample is disclosed herein and also described in U.S. Patent Application Publication 2016/0123990. Antibody binding to Fc receptor can be measured by determining the equilibrium binding constant. Reduction in Fc receptor binding of an antibody is determined by an increase in the equilibrium binding constant for the IgG:FcR interaction.

Methods according to the invention are applicable to both loss of function and gain and function genetic defects. The term “loss-of-function” in reference to a genetic defect as used herein, refers to any mutation in a gene in which the protein encoded by said gene (i.e., the mutant protein) exhibits either a partial or a full loss of function that is normally associated with the wild-type protein. The term “gain-of-function” in reference to a genetic defect as used herein, refers to any mutation in a gene in which the protein encoded by said gene (i.e., the mutant protein) acquires a function not normally associated with the protein (i.e., the wild-type protein) causes or contributes to a disease or disorder. The gain-of-function mutation can be a deletion, addition, or substitution of a nucleotide or nucleotides in the gene, which gives rise to the change in the function of the encoded protein. In certain embodiments, the gain-of-function mutation changes the function of the mutant protein or causes interactions with other proteins. In certain embodiments, the gain-of-function mutation causes a decrease in or removal of normal wild-type protein, for example, by interaction of the altered, mutant protein with said normal, wild-type protein.

Diseases and disorders that may be treated by methods according to the invention include, for example and without limitation, lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, a lysosomal storage disease (e.g., aspartylglucosaminuria, Batten disease, late infantile neuronal ceroid lipofuscinosis type 2 (CLN2), cystinosis, Fabry disease, Gaucher disease types I, II, and III, glycogen storage disease II (Pompe disease), GM2-gangliosidosis type I (Tay Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A, B, C, D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), a disease of solid organs (e.g., brain, liver, kidney, heart), or an infectious viral (e.g., hepatitis B and C, HIV, etc.), bacterial or fungal disease.

Glycogen storage disease type II, also called Pompe disease, may be treated by methods according to the invention. Pompe disease is an autosomal recessive disorder caused by mutations in the gene encoding the lysosomal enzyme acid α-glucosidase (GAA), which catalyzes the degradation of glycogen. The resulting enzyme deficiency leads to pathological accumulation of glycogen and lysosomal alterations in all tissues of the body, resulting in cardiac, respiratory, and skeletal muscle dysfunction (van der Ploeg et al., 2008, Lancet, 372:1342-1353).

Blood clotting disorders which may be treated by methods according to the invention, include, for example and without limitation, hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

Other diseases and disorders that may be treated by methods according to the invention include, for example and without limitation, anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors), or a platelet disorder such as, Bernard Soulier syndrome, Glanzmann thrombasthenia, or storage pool deficiency.

In certain embodiments, the subject has a disease that affects or originates in the central nervous system (CNS) or a neurodegenerative disease, such as, for example and without limitation, Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain embodiments, the CNS or neurodegenerative disease is a polyglutamine repeat disease such as, for example and without limitation, spinocerebellar ataxia (SCA1, SCA2, SCA3, SCA6, SCA7, or SCA17).

The invention may be used in human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects also include animal disease models, for example, mouse and other animal models of protein/enzyme deficiencies such as Pompe disease (loss of GAA), and glycogen storage diseases (GSDs) and others known to those of skill in the art.

The invention provides compositions, such as kits, that include packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, virus (e.g., AAV, lentivirus) vector, or virus particle, an agent that reduces the interaction of IgG with FcRn (e.g., an anti-FcRn antibody, an FcRn binding peptide, an FcRn binding affibody, a small molecule FcRn antagonist), and, optionally, a protease and/or glycosidase that degrades or digests antibodies.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component.

Unless otherwise defined, all technical and scientific terms used herein have the same meaning as commonly understood by one of ordinary skill in the art to which the invention belongs. Although methods and materials similar or equivalent to those described herein can be used in the practice or testing of the invention, suitable methods and materials are described herein.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

The term “about” as used herein refers to a value within 10% of the underlying parameter (i.e., plus or minus 10%). For example, “about 1:10” means 1.1:10.1 or 0.9:9.9, and about 5 hours means 4.5 hours or 5.5 hours, etc. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

Reference to an integer with more (greater) or less than includes any number greater or less than the reference number, respectively. Thus, for example, reference to more than 2 includes 3, 4, 5, 6, 7, 8, 9, 10, 11, 12, 13, 14, 15, etc., and so forth. For example, administration of a recombinant viral vector, protease and/or glycosidase “two or more” times includes 3, 4, 5, 6, 7, 8, 9, 10, 11, 12, 13, 14, 15, or more times.

Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.1 minutes, 1.2 minutes, 1.3 minutes, 1.4 minutes, 1.5 minutes, etc., as well as one day, 2 days, 3 days, 4 days, 5 days . . . 81 days, 82 days, 83 days, 84 days, 85 days, etc., and so forth.

The invention is generally disclosed herein using affirmative language to describe the numerous embodiments of the invention. The invention also specifically includes embodiments in which particular subject matter is excluded, in full or in part, such as substances or materials, method steps and conditions, protocols, or procedures. For example, in certain embodiments of the invention, materials and/or method steps are excluded. Thus, even though the invention is generally not expressed herein in terms of what the invention does not include, aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

### Example 1

Combination Treatment with Anti-FcRn Antibodies and Endopeptidase

The presence and development of IgG specific for AAV capsid protein (anti-capsid IgG) represents a significant challenge to AAV gene therapy, as anti-capsid IgG can inhibit or neutralize AAV transduction of cells and tissues. In an effort to improve gene therapy, it is desirable to reduce and/or remove circulating IgG to enable rAAV dosing of patients that have pre-existing AAV neutralizing antibodies (NAbs), due to natural AAV exposure or due to previous dosing with rAAV.

In a dual approach to overcoming NAbs, a subject is administered an initial treatment regimen of antibodies that bind FcRn and inhibit the interaction of IgG with FcRn (anti-FcRn antibodies), followed by subsequent administration of a regimen of IgG-specific endopeptidase, such as IdeS). It has previously been shown that 3 weekly doses in humans of an anti-FcRn antibody that inhibits FcRn-mediated IgG recycling (M281) can result in up to 80% reduction of IgG in plasma (Ling et al., 2019). The combination of pre-depletion of IgG by anti-FcRn antibodies with IgG cleavage by IdeS administration could potentially overcome significantly higher starting NAb titers than either treatment regimen alone.

Subjects having NAb titers of 1:160 are put in four test groups: 1) untreated; 2) anti-FcRn antibody treatment only; 3) IdeS treatment only; and 4) anti-FcRn antibody and IdeS combined treatment. Anti-FcRn antibody is infused by intravenous, subcutaneous, intraperitoneal or other route of administration with single or multiple ascending doses of 0, 0.3, 3, 10, 30 and 60 mg/kg (and higher doses). Doses of anti-FcRn antibody are administered once weekly (for example) for 1, 2, 3, or 4 weeks (or more), followed by Ides infusion by intravenous, subcutaneous, intraperitoneal or other route of administration with single or multiple ascending doses of 0, 0.5, 1 and 2 mg/kg (and higher doses). NAb titers are assessed before and after each treatment.

### Example 2

### Rabbit Model of AAV Redosing

Rabbits are infused with rAAV vector particles carrying a transgene of interest to induce anti-AAV NAb. After the rabbits develop an anti-AAV NAb titer, they are administered an anti-FcRn antibody that inhibits interaction of IgG with FcRn. Dosing with the anti-FcRn antibody is carried out for 2, 3, 4 or more weeks, at single or multiple ascending doses of 0, 0.3, 3, 10, 30 and 60 mg/kg (and higher doses), for example. After the course of anti-FcRn antibody dosing, IdeS is administered at single or multiple ascending doses of 0, 0.5, 1 and 2 mg/kg (and higher doses). 24 to 48 hours after IdeS administration, rabbits are infused with additional rAAV particles carrying a different transgene. This model allows for analysis of transduction efficacy at varying stages, including before and after anti-FcRn and IdeS treatments and before and after redosing with rAAV.

### Example 3

### Methods

Cleavage of immunoglobulin G (IgG) by endopeptidase in vitro: Human serum or non-human primate (NHP) plasma samples with or without neutralizing antibodies (NAb) to Spk2 capsid were incubated with increasing doses of immunoglobulin-degrading enzyme from Streptococcus pyogenes (IdeS; Promega) for 1 hr at 37° C. As per Promega's indications, one unit is defined as cleaving ≥95% of 1 μg of recombinant monoclonal IgG in 30 min at 37° C. The reaction volume was adjusted with PBS. Cleavage of total IgG was assessed by SDS-PAGE and Coomassie stain.

SDS-PAGE analysis of cleaved IgG: Cleaved samples were prepared for non-reducing SDS-PAGE with NuPAGE® LDS Sample Buffer (4×) (ThermoFisher Scientific) and heated to 70° C. for 10 min. Samples were then analyzed by NuPAGE® Novex 4-12% Bis-Tris gel using MOPS SDS Running Buffer. Gels were stained with Coomassie Blue.

Anti-AAV Capsid Neutralizing Antibody (NAb) Titer: Neutralizing antibodies to AAV-Spk1 or AAV-Spk2 capsid were quantified using a cell-based, in vitro assay and either an AAV-Spk1 or AAV-Spk2, respectively, reporter-vector encapsidating a Renilla luciferase transgene. In brief, early passage (passage less than #26) 293E4 cells were thawed and plated in a flat-bottom white 96-well plate at 2×10⁴ cells/200 μL/well. Ponasterone A (Invitrogen; cat. #H101-01) was added to a final concentration of 1 μg/mL to each well in order to induce expression of the helper virus protein, human adenovirus E4. Cells were then cultured overnight in a 37° C./5% CO₂ incubator. The following day, samples were heat-inactivated at 56° C. for 30 min, then a 4-point dilution (from 1:1 to 1:5) was prepared using fetal bovine serum (FBS) as the diluent. Factor Assay Control Plasma (FACT; King George Bio-Medical, Inc.) was prepared in a 3.16-fold (half-log) serial dilution to assess assay performance. AAV-luciferase vector was diluted to 7.5×10⁷ vg/mL in DMEM and then added to FACT controls and samples. Vector and controls/samples were incubated at 37° C. for 60 min. Volumes 7.5 μL per well of the “neutralized” controls/samples were transferred to each well of the plate seeded with cells, and the cells were returned to the incubator for overnight incubation. The next day cells were washed once in PBS, lysed in Renilla Assay Lysis Buffer, and luciferase activity was measured with the Renilla Luciferase Assay System (Promega) and read on a SpectraMax® L microplate reader.

Artificial Immunization Mouse Model Using IVIg: To create an artificial NAb titer in male C57BL/6 mice, intravenous immune globulin (IVIg; Gamunex), containing 10% immunoglobulin G (IgG) purified from human blood, was injected intraperitoneally (IP) one day prior to intravenous (IV) vector administration. To determine whether in vitro cleavage of IgG by IdeS, or IdeZ—a similar endopeptidase from Streptococcus equi, which has improved activity against mouse IgG2a and IgG3 compared to IdeS—can rescue transduction efficiency in vivo, IVIg was treated with 125 units of IdeZ (Promega) overnight at 37° C. prior to IP dosing. Cleavage of total IgG was assessed by SDS-PAGE and Coomassie stain. One day post IVIg dose, vector (AAV-Spk1-GAA) was administered at 2×10¹² vg/kg. Mouse plasma samples were collected weekly, and samples were analyzed for activity of the transgene (GAA enzyme). To determine whether in vivo cleavage of IgG by IdeS can rescue transduction efficiency in vivo, 300 mg/kg IVIg was infused by IP dosing. After 24 hours, either 0.4 or 4 mg/kg IdeS was infused by IV dosing. Then 24 hours after IdeS infusion, vector (AAV-Spk1-GAA) was administered at 2×10¹² vg/kg.

GAA Activity Assay: GAA activity was assessed by measurement of cleavage of the substrate 4-methyl-umbelliferyl-α-D-glucoside at pH 4 (Galjaard et al., Clin Chim Acta 1973; 49(3):361-75). Briefly, the reaction was initiated by addition of 20 μL of substrate to 10 μL plasma sample diluted 1:250 in MilliQ water. The reaction mixture was incubated at 37° C. for 1 hr and then subsequently stopped by carbonate buffer at pH 10.5. The standard curve was plated thereafter with 4-methylumbelliferone, the blue fluorescent dye liberated from 4-methyl-umbelliferyl-α-D-glucoside, which produces a fluorescent emission at 440 nm when excited at 370 nm.

Anti-AAV Capsid IgG antibodies: Anti-AAV capsid total IgG formation was measured with a capture assay. ELISA plate wells were coated with 50 μL of a solution containing 1 μg/mL of AAV-Spk1 capsid particles. Total human IgG (Southern Biotech, 0150-01) was diluted to generate a 10-point standard curve ranging from 10,000 ng/mL to 0.5 ng/mL and added to the plate. The limit of quantitation of the assay was 460 ng/mL after back-calculation. Three levels of quality control samples were prepared and included on each plate to assess assay performance. Capsid particles, standards, and quality controls (QCs) were incubated overnight at 4° C. After washing, wells were blocked with 2% BSA, 0.05% Tween-20 in PBS for 2 hrs at room temperature. Then, serial dilutions of samples in blocking buffer were loaded on the plate and incubated at room temperature for 2 hours. A horseradish peroxidase (HRP)-conjugated sheep anti-human IgG antibody (GE Healthcare, cat. #NA933V) diluted 1:5000 in blocking buffer was used as detecting antibody and incubated on the plate for 1 hr at room temperature. Following washing, the peroxidase activity was revealed following a 10-minute incubation at room temperature with 3,3′,5,5′-tetramethylbenzidine substrate (TMB). The reaction was stopped with 1M sulfuric acid, and then the plate was read by an absorbance plate reader for optical density (OD) at 450 nm. IgG concentration was determined against a standard curve made with serial dilution of purified human total IgG.

### Example 4

IdeS Cleaves IgG from Human, NHP and Hamster Samples In Vitro

The immunoglobulin G (IgG)-degrading enzyme from Streptococcus pyogenes (IdeS) is a cysteine protease that cleaves all four human subclasses of IgG with high specificity. IdeS hydrolyzes human IgG at Gly236 in the lower hinge region of the IgG heavy chains.

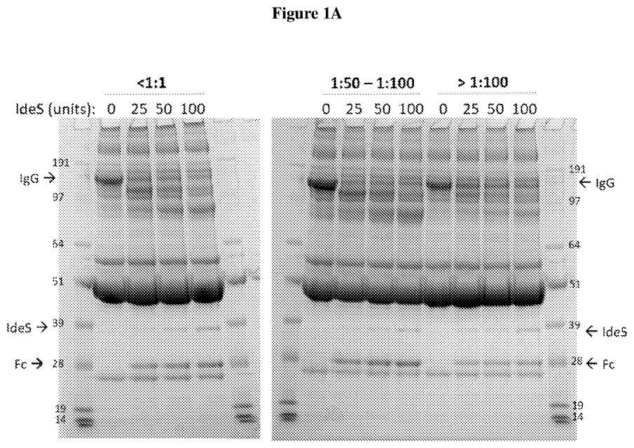

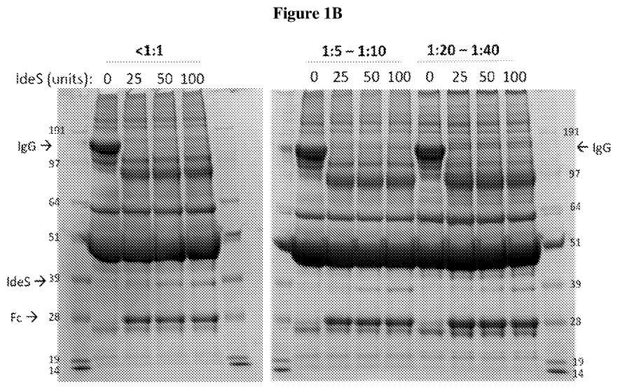

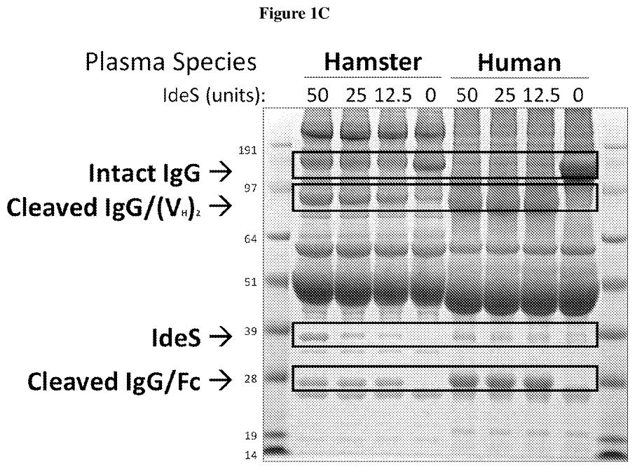

To analyze the ability of IdeS to cleave IgG in serum, increasing doses of IdeS (0-100 units; Promega) were added to human serum and NHP (rhesus macaque) plasma samples with or without an anti-Spk2 NAb titer. In human samples that were naïve (<1:1) or had relative mid-range (1:5-1:10) or relative high (1:20-1:40) NAb titers, the lowest dose of IdeS cleaved all total IgG (˜150 kD) to liberate the Fc fragment (˜25 kD) (FIG. 1A). In the NHP samples, IgG was similarly cleaved in all NAb titer groups (<1:1, 1:50-1:100, >1:100, FIG. 1B).

Hamsters are expected to provide a better model than mice for examining IdeS treatment for AAV redosing. IdeS was tested for the ability to cleave hamster IgG, by incubating increasing amounts of IdeS (0 to 50 units; Promega), with pooled hamster plasma (and pooled human plasma as a positive control). Samples were analyzed by non-reducing SDS-PAGE and Coomassie Blue staining. IdeS was effective to cleave the IgG in the pooled hamster plasma and pooled human plasma in vitro (FIG. 1C).

These results demonstrate that IdeS is a highly efficient and specific protease of human IgG, rhesus IgG and hamster IgG.

### Example 5

### Cleavage of IgG by IdeS Results in a Reduced NAb Titer In Vitro

To analyze whether cleavage of IgG by IdeS is sufficient to reduce neutralization, AAV vector transduction efficiency was assayed in vitro. In this assay, serum or plasma samples from various species can be assessed for the presence of neutralizing antibodies to the AAV capsid by pre-incubating AAV vectors encoding Renilla luciferase with plasma or sera, transducing human cells in culture with these mixtures, and subsequently assessing levels of luciferase activity.

Human patient serum samples that were naïve (<1:1) or had a high anti-Spk2 NAb titer (1:10-1:20) were pretreated with or without excess IdeS (50 units), and then NAb titers were assessed. Interestingly, the patient sample with a previously-reported NAb titer of 1:10-1:20 showed at least a two-fold decrease with IdeS pretreatment to a 1:5-1:10 NAb titer in one study

(Table 1). These results demonstrate that AAV vector transduction is increased when IgG antibodies are cleaved by IdeS.

TABLE 1
Anti-Spk2 NAb titer analysis of human
sera incubated with excess IdeS.

| | | | | | |
|---|---|---|---|---|---|
| Sample | | | | Previous | Titer |
| # | Spark ID | IdeS | Vector | Titer | Range |

Study 1

| | | | | | |
|---|---|---|---|---|---|
| FACT | NA | NA | NA | NA | 1:100-1:316 |
| Plate 1 | | | | | |
| S1 | 397 | No | Spk2 | <1:1 | <1:1 |
| S2 | 427 | No | Spk2 | 1:10-1:20 | 1:10-1:20 |
| S3 | 397 | Yes | Spk2 | <1:1 | <1:1 |
| S4 | 427 | Yes | Spk2 | 1:10-1:20 | 1:5-1:10 |

Study 2

| | | | | | |
|---|---|---|---|---|---|
| S1 | FBS | No | Spk2 | NA | <1:1 |
| S2 | FBS | Yes | Spk2 | NA | <1:1 |
| S3 | BRH1450399 | No | Spk2 | 1:2.5 | 1:2.5 |
| S4 | BRH1450399 | Yes | Spk2 | 1:2.5 | 1:1 |
| S5 | BRH1450436 | No | Spk2 | ~1:5 | 1:5 |
| S6 | BRH1450436 | Yes | Spk2 | ~1:5 | 1:1 |
| S7 | BRH1450427 | No | Spk2 | 1:10 | 1:10 |
| S8 | BRH1450427 | Yes | Spk2 | 1:10 | 1:10 |

Anti-Spk2 NAb titer analysis following treatment with IdeS endopeptidase. Human patient samples (designated by Spark ID) were pretreated with and without IdeS. NAb titers were later assessed by in vitro vector transduction assay.

### Example 6

### Degradation of IVIg by IdeZ In Vitro Increases the Transduction Efficiency of Vector In Vivo

The effector functions of IgG antibodies, such as cytotoxicity and complement fixation, are mediated by the Fc portion. Neutralization relies on the variable regions of the heavy and light chains for specificity to antigen. While the F(ab′)₂ fragment still contains intact antigen-binding regions, data suggest that liberation of the F(ab′)₂ fragment by IdeS or IdeZ, a similar endopeptidase in Streptococcus equi, which has improved activity against mouse IgG2a and IgG3, causes reduced stability without the Fc portion and therefore quicker clearance of the F(ab′)₂ fragment from circulation than intact IgG. This assay tested whether administration of neutralizing antibodies that were pre-cleaved by IdeS or IdeZ should result in a reduction of neutralizing activity to AAV in the in vivo setting.

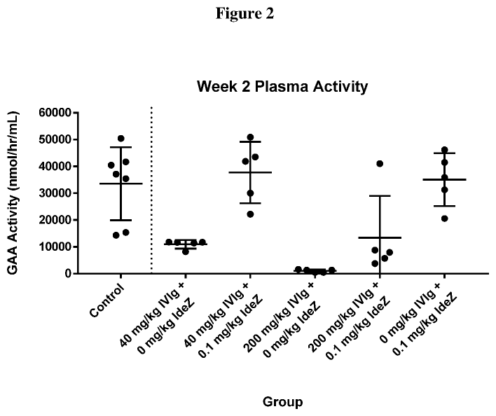

Mice were immunized with IVIg, a pool of human IgGs that includes anti-AAV capsid neutralizing antibodies that were pretreated with or without 0.1 mg/kg IdeZ. Mice were then administered 2×10¹² vg/kg AAV-Spk1-GAA. Mice treated with 1.0 mg or 5.0 mg IVIg resulted in reduced GAA activity levels in plasma (10,951±1,554 nmol/hr/mL and 1,041±553 nmol/hr/mL respectively) compared with control mice (33,551±13,635 nmol/hr/mL) showing that vector neutralization by IVIg is dose dependent (FIG. 2).

Pretreatment of 40 mg/kg IVIg with IdeZ rescued transduction efficiency, resulting in GAA activity levels (37,707±11,449 nmol/hr/mL) that were comparable to control. IdeZ pretreatment of 200 mg/kg IVIg partially alleviated vector neutralization (13,440 nmol/hr/mL±15,543) with one animal completely recovering activity (41,025 nmol/hr/mL). Of note, IdeZ itself did not interfere with AAV vector transduction efficiency. IVIg dose retains were analyzed by SDS-PAGE with Coomassie stain to confirm cleavage of IgG. These results indicate that in vitro cleavage of neutralizing antibodies to the AAV capsid by IdeS/IdeZ can rescue AAV vector transduction and transgene expression in vivo.

### Example 7

### Degradation of IVIg by IdeS In Vivo Increases the Transduction Efficiency of Vector In Vivo

To analyze whether cleavage of IgG in vivo can affect vector transduction and transgene expression/activity in plasma, mice were first infused with intact IVIg to create an artificial titer of human anti-capsid neutralizing IgGs. After 24 hours, mice were infused with IdeS at two concentrations (0.4 mg/kg or 4 mg/kg), and then 24 hours after IdeS infusion, all mice were administered 2×10¹² vg/kg AAV-Spk1-GAA. Both anti-Spk1 NAb titers and IgG levels were analyzed pre-IdeS infusion and post-IdeS infusion (immediately prior to vector administration).

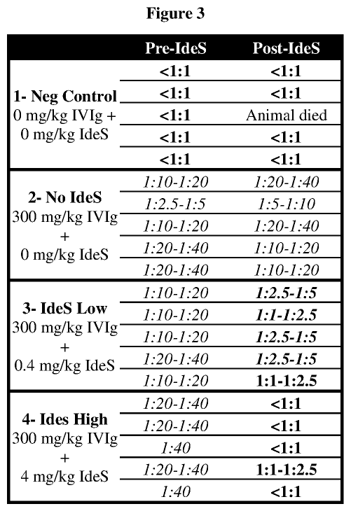

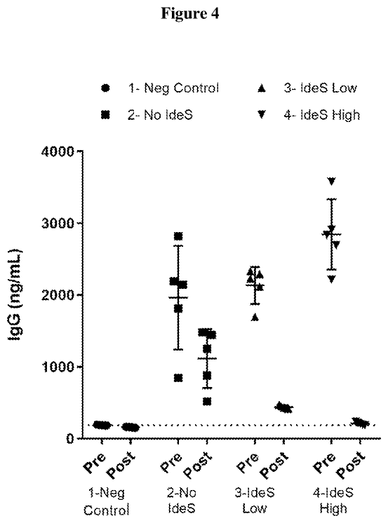

IdeS infusion induced a dose-dependent decrease in both NAb (FIG. 3) and IgG levels (FIG. 4). The highest dose of IdeS (4 mg/kg) was capable of reducing NAb titers of at least 1:40 down to <1:1.

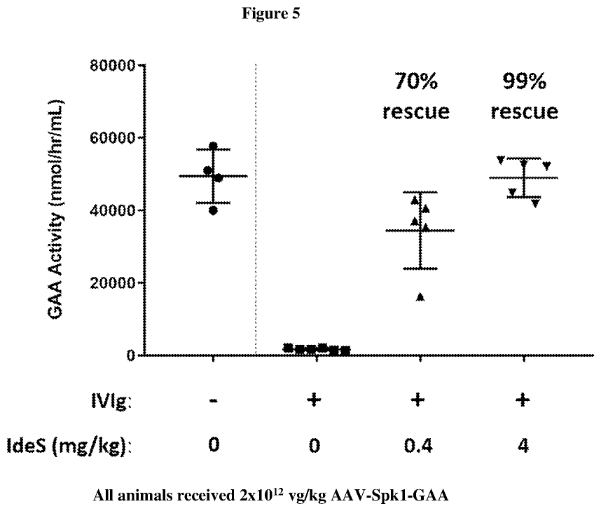

When GAA activity was measured one week post vector infusion, control mice administered only vector demonstrated GAA activity levels of 49,387±7,345 nmol/hr/mL (FIG. 5). Mice that were injected with 300 mg/kg IVIg exhibited GAA transgene activity levels in plasma (1,702±336 nmol/hr/mL) consistent with almost complete inhibition of transduction. IdeS displayed a dose-dependent rescue of transgene activity levels; 0.4 mg/kg IdeS resulted in a 70% rescue of GAA activity (34,408±10,562 nmol/hr/mL), while 4 mg/kg IdeS rescued 99% GAA activity (48,948±5,322 nmol/hr/mL). These results demonstrate that IdeS treatment in vivo reduces neutralizing antibody titers and allows for dosing and transduction of AAV vectors in animals that are refractory to treatment.

### Example 8

### Degradation of IVIg by IdeS In Vivo Increases the Transduction Efficiency of Vector In Vivo

IdeS was evaluated for ability to cleave higher titers of anti-capsid IgG in vivo and to rescue AAV transduction in the context of a higher degree of AAV vector neutralization. Mice (male C57BL/6) were infused with varying doses of intact human IVIg (300 mg/kg (low), 800 mg/kg (mid), or 1600 mg/kg (high)) to create an artificial titer of human anti-capsid neutralizing IgGs. After 24 hours, mice were infused with IdeS at three concentrations (0.4 mg/kg (low), 1 mg/kg (mid), or 2 mg/kg (high)). 24 hours after IdeS infusion, mice were administered AAV-Spk1-GAA at 2×10¹² vg/kg. Anti-Spk1 NAb titers were determined at both pre-IdeS infusion and post-IdeS infusion (immediately prior to vector administration), using the Anti-AAV Capsid NAb Titer assay described in Example 1. AAV transduction was assessed by measurement of transgene product (GAA) activity in plasma using the GAA Activity Assay, as described in Example 1, two weeks post vector administration.

For all doses of IVIg, pre-treatment with IdeS yielded a dose-dependent decrease in AAV NAb titer (Table 2). Table 2 presents the neutralizing anti-Spk1 antibody (NAb) titer pre- and post-IdeS infusion for each animal in each group. AAV NAb titers are designated as low (<1:1, 1:1-1:2.5), low-to-mid range (1:2.5-1:5), mid-to-high range (1:5-1:10) and high (>1:10-1:20). The highest dose of IdeS (2 mg/kg) was capable of reducing NAb titers of >1:160 (generated with 1600 mg/kg IVIg) down to 1:1-1:2.5.

TABLE 2
Anti-NAb titers in murine plasma pre- and post-IdeS infusion.

| | Pre-IdeS | Post-IdeS |
|---|---|---|
| Negative Control | <1:1 | <1:1 |
| 0 mg/kg IVIg + | <1:1 | <1:1 |
| 0 mg/kg IdeS | <1:1 | <1:1 |
| | <1:1 | <1:1 |
| | <1:1 | <1:1 |
| | <1:1 | <1:1 |
| | <1:1 | <1:1 |
| Low IVIg + No IdeS | 1:20-1:40 | 1:20-1:40 |
| 300 mg/kg IVIg + | 1:40 | 1:10-1:20 |
| 0 mg/kg IdeS | 1-20-1:40 | 1:20-1:40 |
| | 1:40-1:80 | 1:10-1:20 |
| | 1:10-1:20 | 1:20-1:40 |
| Low IVIg + Low IdeS | 1:20-1:40 | 1:2.5-1:5 |
| 300 mg/kg IVIg + | 1:20-1:40 | 1:1-1:2.5 |
| 0.4 mg/kg IdeS | 1:20-1:40 | 1:40-1:80 |
| | 1:20-1:40 | 1:1-1:2.5 |
| | 1:20-1:40 | 1:2.5 |
| Low IVIg + Mid IdeS | 1:40-1:80 | 1:1-1:2.5 |
| 300 mg/kg IVIg + | 1:80-1:160 | <1:1 |
| 1.0 mg/kg IdeS | 1:80-1:160 | 1:1-1:2.5 |
| | 1:20-1:40 | 1:1-1:2.5 |
| | 1:20-1:40 | 1:1-1:2.5 |
| Low IVIg + High IdeS | 1:20-1:40 | <1:1 |
| 300 mg/kg IVIg + | 1:10-1:20 | <1:1 |
| 2.0 mg/kg IdeS | 1:20-1:40 | <1:1 |
| | 1:20-1:40 | <1:1 |
| | 1:20-1:40 | <1:1 |
| Mid IVIg + No IdeS | 1:40-1:80 | 1:20-1:40 |
| 800 mg/kg IVIg + | 1:40-1:80 | 1:80-1:160 |
| 0 mg/kg IdeS | 1:40-1:80 | 1:40-1:80 |
| | 1:40-1:80 | 1:40-1:80 |
| | 1:40-1:80 | 1:40 |
| Mid IVIg + Low IdeS | 1:40-1:80 | 1:5-1:10 |
| 800 mg/kg IVIg + | 1:40-1:80 | 1:5-1:10 |
| 0.4 mg/kg IdeS | 1:20-1:40 | 1:2.5 |
| | 1:20-1:40 | 1:2.5-1:5 |
| | 1:40-1:80 | 1:5-1:10 |
| Mid IVIg + Mid IdeS | 1:80-1:160 | 1:2.5-1:5 |
| 800 mg/kg IVIg + | 1:40-1:80 | 1:1-1:2.5 |
| 1.0 mg/kg IdeS | 1:40-1:80 | <1:1 |
| | 1:80-1:160 | 1:1-1:2.5 |
| | 1:40-1:80 | 1:1-1:2.5 |
| Mid IVIg + High IdeS | 1:40-1:80 | <1:1 |
| 800 mg/kg IVIg + | 1:40-1:80 | <1:1 |
| 2.0 mg/kg IdeS | 1:80-1:160 | <1:1 |
| | 1:1-1:2.5 | <1:1 |
| | 1:20-1:40 | <1:1 |
| | 1:80 | 1:1-1:2.5 |
| High IVIg + No IdeS | >1:160 | 1:80-1:160 |
| 1600 mg/kg IVIg + | >1:160 | 1:40-1:80 |
| 0 mg/kg IdeS | >1:160 | 1:40-1:80 |
| | >1:160 | 1:40-1:80 |
| | >1:160 | 1:40-1:80 |
| | >1:160 | 1:40-1:80 |
| High IVIg + Low IdeS | 1:1-1:2.5 | 1:1-1:2.5 |
| 1600 mg/kg IVIg + | >1:160 | 1:5-1:10 |
| 0.4 mg/kg IdeS | 1:20-1:40 | 1:2.5-1:5 |
| | >1:160 | 1:10-1:20 |
| | >1:160 | 1:10-1:20 |
| High IVIg + Mid IdeS | 1:40-1:80 | 1:1-1:2.5 |
| 1600 mg/kg IVIg + | >1:160 | 1:2.5-1:5 |
| 1.0 mg/kg IdeS | >1:160 | 1:2.5-1:5 |
| | >1:160 | 1:2.5-1:5 |
| | >1:160 | 1:2.5-1:5 |
| High IVIg + High IdeS | >1:160 | 1:2.5-1:5 |
| 1600 mg/kg IVIg + | >1:160 | 1:1-1:2.5 |
| 2.0 mg/kg IdeS | >1:160 | 1:1-1:2.5 |
| | >1:160 | 1:1-1:2.5 |
| | <1:1 | <1:1 |

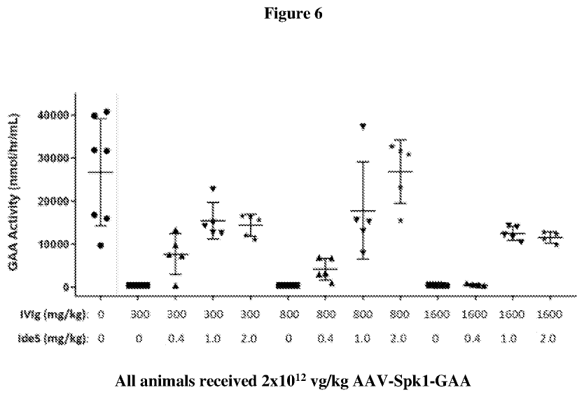

The results for GAA activity, measured two weeks post vector infusion, are shown in FIG. 6. The plasma of negative control mice (administered vector only) demonstrated GAA activity levels of 26,689±12,420 nmol/hr/mL. Mice injected with 300 mg/kg (and higher) IVIg, and receiving no IdeS, exhibited plasma GAA activity levels of only 436±41 nmol/hr/mL, consistent with NAb inhibition of AAV vector transduction. Consistent with the results in the previous Examples, IdeS pre-treatment resulted in a dose-dependent rescue of AAV vector transduction, as measured by GAA activity levels in plasma: 0.4 mg/kg IdeS resulted in GAA activity levels of 7,702±4,710 nmol/hr/mL, 1 mg/kg IdeS resulted in GAA activity levels of 15,444±4,226 nmol/hr/mL, and 2 mg/kg IdeS resulted in GAA activity levels of 14,375±2,572 nmol/hr/mL.

Groups that received higher doses of IVIg (either 800 or 1600 mg/kg IVIg) demonstrated similar trends of a dose-dependent increase in GAA activity levels with increasing IdeS. With 800 mg/kg IVIg, 0.4 mg/kg IdeS resulted in GAA activity levels of 4,188±2,549 nmol/hr/mL, 1 mg/kg IdeS resulted in GAA activity levels of 17,813±11,283 nmol/hr/mL, and 2 mg/kg IdeS resulted in GAA activity levels of 26,846±7,354 nmol/hr/mL. With 1600 mg/kg IVIg, 0.4 mg/kg IdeS resulted in GAA activity levels of 580±217 nmol/hr/mL, 1 mg/kg IdeS resulted in GAA activity levels of 12,511±1,602 nmol/hr/mL and 2 mg/kg IdeS resulted in GAA activity levels of 11,573±1,313 nmol/hr/mL. At the highest dose of IVIg (1600 mg/kg), the lowest IdeS dose (0.4 mg/kg) failed to rescue vector transduction. At the 1600 mg/kg dose of IVIg, however, there are supraphysiological levels of total IgG present in circulation, which likely reduced the effectiveness of IdeS at the 0.4 mg/kg dose due to the increased amount of its substrate.

These results show that IdeS treatment in vivo reduces neutralizing antibody titers and allows for dosing and transduction of viral vectors in animals that are refractory to viral vector gene therapy treatment methods.

### Example 9

### Hamster Model of AAV Redosing

Hamsters are infused with rAAV vector particles carrying a transgene of interest, are dosed with IdeS after the development of anti-AAV NAb (e.g., 4 weeks), and infused with additional rAAV particles carrying another transgene. This model allows for analysis of transduction efficacy at varying stages, including before and after IdeS treatment and before and after redosing with rAAV.

To assess the ability of IdeS to degrade or reduce the effects of neutralizing antibodies to the AAV capsid, a study is performed in Syrian Golden hamsters, a species whose IgG is efficiently cleaved by the endopeptidase IdeS. Hamsters are first infused with 2×10¹² vg/kg Spk1-FVIII, Spk1-FIX, or other Spk1 encapsidated vector. Animals are monitored for expression of the transgene product (i.e., FVIII, FIX, etc.) in plasma, in addition to measurement of the development of NAbs to the Spk1 capsid by anti-Spk1 IgG ELISA or a cell-based neutralizing antibody assay. Following the development of NAb titer, within 3-5 weeks of vector infusion, animals are infused by intravenous, subcutaneous, intraperitoneal or other route of administration with single or multiple ascending doses of IdeS of 0, 0.5, 1, and 2 mg/kg (and higher doses). After IdeS administration, animals are followed by measuring anti-Spk1 capsid IgG and/or NAbs to Spk1. When animals display a sufficient decrease in NAb levels, they are infused with 2×10¹² vg/kg Spk1-GAA. Following transduction, GAA expression is measured in plasma by GAA activity assay and/or GAA antigen level measurement to determine the level of transduction attained.

These studies show if IdeS reduces AAV capsid-specific NAbs in vivo to a level low enough to enable redosing. Measurement of anti-FVIII IgG (if developed in vivo), provides information regarding the effectiveness of IdeS in reducing transgene product-targeting NAbs, and the number of rounds of IdeS redosing permissible before loss of effectiveness.

### Example 10

### Cynomolgus Monkey Model of AAV Redosing

To assess the ability of IdeS to degrade or reduce the effects of NAbs against the AAV capsid in a large animal model, a study is performed in cynomolgus monkeys (Macaca fascicularis). Monkeys are first screened for pre-existing NAbs to the Spk1 capsid. NAb positive animals likely result from exposure to naturally occurring AAV in the wild or in group housing. Animals are placed into groups based on negative or positive NAb titer, and, if positive, how high the pre-existing NAb titer is.

In the re-dosing arm of the study, animals are dosed with 2×10¹² vg/kg Spk1-FIX, or other Spk1 encapsidated vector. Animals are monitored for expression of the transgene product (i.e., FIX or other) in plasma, in addition to measurement of the development of NAbs to the Spk1 capsid by anti-Spk1 IgG ELISA or a cell-based NAb assay. Following development of NAb titer, within 3-5 weeks of vector infusion, animals are infused by intravenous, subcutaneous, intraperitoneal or other route of administration with single or multiple ascending doses of IdeS of 0, 0.5, 1, and 2 mg/kg, and higher doses. After IdeS administration, animals are followed by measuring anti-Spk1 capsid IgG and/or NAbs to Spk1. When animals display a sufficient decrease in NAb levels, they are infused with 2×10¹² vg/kg Spk1-GAA. Following transduction, GAA expression is measured in plasma by GAA activity assay and/or GAA antigen level assessment to determine the level of transduction attained.

A separate arm of the study evaluates the ability of IdeS to overcome pre-existing NAb titers. Animals displaying different NAb titer levels are grouped based on titer and infused by intravenous, subcutaneous, intraperitoneal or other route of administration with single or multiple ascending doses of IdeS of 0, 0.5, 1, and 2 mg/kg (and higher doses). Following IdeS administration, animals are followed by measuring anti-Spk1 capsid IgG and/or NAbs to Spk1 and, when animals display a sufficient decrease in NAb levels, they are infused with 2×10¹² vg/kg Spk1-GAA. Following transduction, GAA expression is measured in plasma by GAA activity assay and/or GAA antigen level measurement to determine the level of transduction attained.

These studies show if IdeS reduces AAV capsid-specific NAbs in vivo to a level low enough to enable redosing in cynomolgus monkeys, a species that is an excellent model of human AAV administration. These studies also show the maximal pre-existing NAb titer that can be overcome by IdeS administration. Measurement of anti-FIX IgG (if developed in vivo), provides information regarding the effectiveness of IdeS in reducing transgene product-targeting NAbs, and the number of rounds of IdeS redosing permissible before loss of effectiveness.

### Example 11

Mouse Study with IdeS and AAV-Spk1-hFVIII

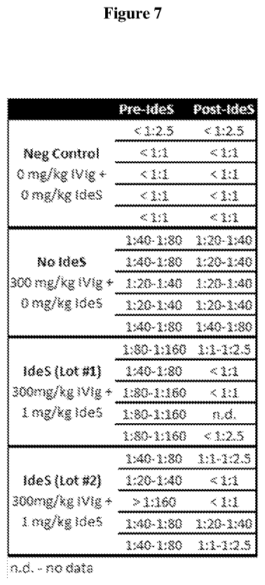

Two different preparations of IdeS (Lot 1 and Lot 2) were tested in mice having an artificial titer of human anti-capsid neutralizing IgGs. C57BL/6 mice were injected with 300 mg/kg of IVIg at Day −2, followed by 1 mg/kg IdeS at Day −1 (pre-dosing with AAV), and finally with 5×10¹⁰ vector genomes of an AAV-Spk1 vector encoding a human Factor VIII (AAV-Spk1-hFVIII) at Day 0 (post-dose). Negative control animals received no IVIg or IdeS treatment, and the “No IdeS” group received only IVIg and AAV-Spk1-hFVIII vector. Neutralizing antibody titers in plasma were determined pre- and post-IdeS administration, using an anti-AAV capsid neutralizing assay similar to that described in Example 3, using an 8-point titer (1:1 to 1:160) on the samples, and luminescence was read on a GloMax® Discover Microplate Reader (Promega). Titer was determined as the highest dilution or range where luminescence was inhibited by >50%. NAb titers pre- and post-IdeS treatment shows that both lots of IdeS were effective in decreasing the NAb titer in the mice (FIG. 7).

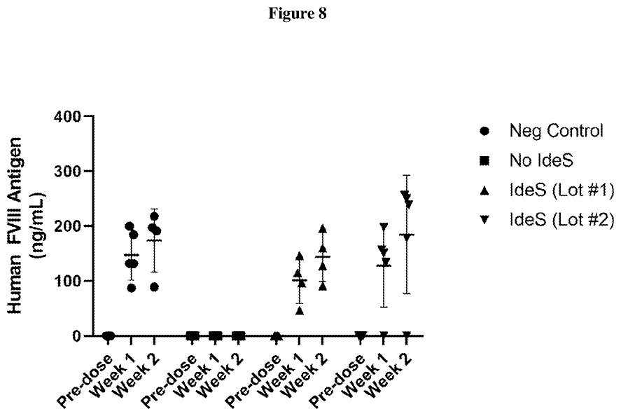

Human FVIII antigen levels were measured by ELISA pre-vector infusion and at one and two weeks post vector infusion (FIG. 8). Both lots of IdeS treatment in vivo reduced neutralizing antibody titers and to allow for dosing with an AAV vector and expression of the transgene.

### Example 12

Mouse Study with Anti-AAV-Spk1 IgG

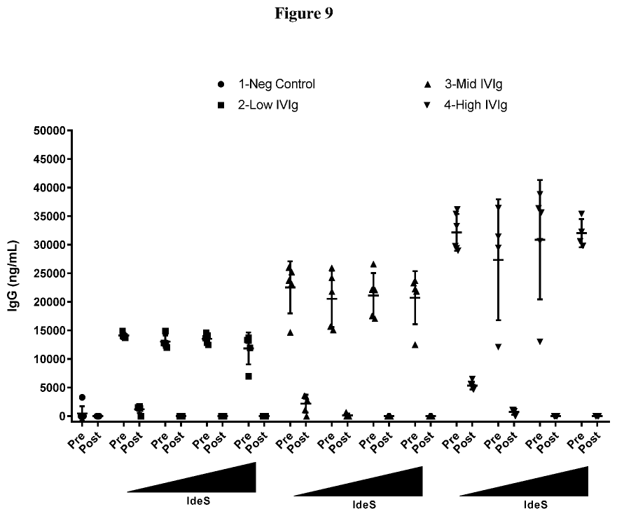

C57BL/6 mice were given IVIg to induce an artificial titer of human anti-capsid neutralizing IgG. Three concentrations of IVIg (300 mg/kg (low), 800 mg/kg (mid), and 1600 mg/kg (high) were used, and within each IVIg group, animals were treated with increasing doses of IdeS (0, 0.4, 1.0, 2.0 mg/kg). Anti-Spk1 capsid IgG levels were assessed by ELISA. Briefly, 96-well plates were coated with Spk1 empty capsid, then blocked with BSA, washed, and incubated with plasma, diluted 1:100, for 2 hours. Following incubation, plates were washed and incubated with a secondary antibody conjugated with HRP for 1 hour. Subsequently, plates were washed again and developed using TMB substrate. Plates were read on an absorbance plate reader for optical density (OD) at 450 nm. Luminescence was compared to a standard curve of human IgG to determine antibody concentrations. All three concentrations of IdeS (0.4, 1.0, 2.0 mg/kg) eliminated or significantly reduced serum levels of anti-Spk1 capsid IgG for all three concentrations (low, mid and high) of IVIg (FIG. 9).

### Example 13

Mouse Study with Anti-FcRn Antibody and Endopeptidase

A study was performed in male Tg32 mice to evaluate the ability of anti-FcRn monoclonal M281 and IgG cleaving endopeptidase IdeS, alone and in combination, to reduce neutralizing antibodies to AAV.

The Tg32 mouse strain (also called hFcRn Tg32 or FcRn−/− hFcRn line 32 Tg), is a standard for evaluating the pharmacokinetics and pharmacodynamics of human IgG and Fc-domain based therapeutics, and carries a knock-out mutation for the mouse Fcgrt (Fc receptor, IgG, alpha chain transporter) gene and a transgene expressing the human FCGRT gene under the control of its own native promoter (hTg32), in the C57BL/6J background (Jackson Laboratory; stock #014565).

Eleven male Tg32 mice per group were pre-immunized with IVIg (to introduce neutralizing antibodies) or Dulbecco's PBS (DPBS) via intraperitoneal injection (Day −1). Mice were injected with M281 or PBS 24 hours later (Day 0), IdeS (Promega) or PBS on Day 1, and Spk1-FIX (Spk1 encapsidated liver-specific promoter FIX expression cassette) AAV particles on Day 2 (Table 3). Mice were bled to collect plasma at 0 and 2 days after IVIg or PBS injection, for NAb and anti-Spk1 IgG analysis. See Table 4 for detailed dosing and collection timeline for the study.

TABLE 3
Groups and treatments.

| | IVIg | | Treatment | Dose | Vector | |
|---|---|---|---|---|---|---|
| Group | Dose | N | (Route) | (mg/kg) | (Route) | Dose |
| 1 | 500 | 11 | PBS (IV) | — | Spk1- | 5 × 10⁵ |
| 2 | mg/kg | 11 | M281 (IV) | 20 | FIX | vg/mouse |
| 3 | | 11 | IdeS (RO) | 0.4 | (IV) | |
| 4 | | 11 | M281 (IV) + | 20 + 0.4 | | |
| | | | IdeS (RO) | | | |
| 5 | PBS | 11 | PBS (IV) | — | | |

N = number of mice in group;
IV = intravenous;
RO = retroorbital;
vg = vector genomes

TABLE 4
Dosing and collection timeline.

| Study Day | Task |
|---|---|
| Day −1 | Body weight measurement |
| | IVIg intraperitoneal (IP) dosing |
| Day 0 | Blood collection |
| | PBS and M281 tail vein intravenous (IV) dosing |
| Day 1 | PBS and IdeS retro orbital IV dosing |
| Day 2 | Body weight measurement |
| | Blood collection |
| | Spk1-FIX tail vein IV dosing |
| Day 4, 7, 9, 11, | Body weight measurement |
| 14, 16, 18, 21 | |
| Day 9, 16 | Blood collection |
| Day 23 | Body weight measurement |
| | Termination: |
| | Terminal blood collection. |
| | Whole liver collection. |
| | Peripheral lymph nodes collection. |

### Methods

Blood Sampling: On days 0, 2, 9, and 16: ˜200 μL whole blood was collected via the submandibular vein and collected on ice. The first drop of blood was discarded, and 100 μL plasma samples were obtained by centrifuging blood at 9800 g, 4° C. for 10 minutes, and aliquoting the supernatant into clean tubes. Plasma samples were stored at −80° C. prior to analysis.

In vitro neutralizing antibody analysis: Neutralizing antibody titers to AAV-Spk1 capsid were determined in vitro. Briefly, HEK-293-E4 cells were seeded onto 96-well plates (Corning, Cat #3595) at 20,000 cells per well. The cells were cultured overnight in a 37° C. incubator with humidified atmosphere of 5% CO₂ using growth media (DMEM, 10% FBS, 2 mM L-Glutamine, 1×Pen/Strep) supplemented with 1 μg/mL Ponasterone A (Fisher Scientific, Cat. #H10101) to induce expression of the human adenovirus protein E4. The following day, all plasma samples were heat-inactivated at 56° C. for 30 minutes using a water bath. Samples were diluted and tested in heat-inactivated FBS in a 2-fold serial dilution to assess inhibitory activity spanning from 1:2.5 to 1:160 dilution. A Factor Assay Control Plasma (FACT) was utilized to assess assay performance between runs. Diluted plasma samples were pre-mixed with a Spk1-Renilla-luciferase AAV reporter virus at a concentration of 1.5×10⁹ vg/mL, and incubated at 37° C. for 1 hour (80 μL total volume). After incubation, 7.5 μL of the pre-mixed plasma/AAV solution was added to triplicate wells the HEK-293-E4 cells and incubated overnight at 37° C./5% CO₂. The following day, 40 μL of Renilla luciferase lysis buffer was added to the cells in each well and luminescence assayed using a GloMax® luminometer. Neutralizing antibody titers were reported as the lowest plasma dilution that resulted in greater than 50% reduction in luminescence when compared to naïve serum (FBS only).

Anti-SPK1 Capsid IgG ELISA Analysis: The presence of anti-capsid IgG in mouse plasma was assessed using a human anti-AAV Spk1 capsid IgG capture assay. Briefly, ELISA plate wells were coated overnight at 4° C. with human IgG standards, quality control (QC) samples, or Spk1 capsid (plasma sample wells). The standard curve consisted of a 10-point, 3-fold dilution series ranging from 10 μg/mL to 4.57 ng/mL, with the top two and bottom two points serving as anchor points. High quality control (HQC), medium quality control (MQC), and low quality control (LQC) samples consisted of human IgG at 800 ng/mL, 500 ng/mL, and 12 ng/mL. After the overnight coating step, plates were washed three times using a plate washer followed by blocking with dilution buffer for 2 hours at room temperature. The plates were washed as above, and diluted plasma samples (1:100) were added to the appropriate wells (dilution buffer alone was added to standard and QC wells). The plates were incubated for 2 hours at room temperature and washed as before. Horseradish peroxidase (HRP) conjugated sheep-anti-human IgG antibody was added to the plate wells and incubated for 1 hour at room temperature. After the incubation, the plates were washed as before. TMB, equilibrated to room temperature, was added to the plates to develop signal detection. After incubation for 10 minutes in the dark at room temperature, signal development was stopped by addition of 1M sulfuric acid. Signal was detected by measuring absorbance at 450 nm using a SpectraMax® plate reader. Assay acceptance criteria were based on the results of standards and QC samples. Anti-capsid IgG levels in plasma samples were interpolated by comparison to the standard curve. Samples which measured <457 ng/mL were defined as below the limit of quantification (BQL). Samples below the detectable absorbance range were assigned a value of 152 ng/mL, which is at the lower limit of detection (LOD).

### Analysis of Neutralizing Antibody Titers

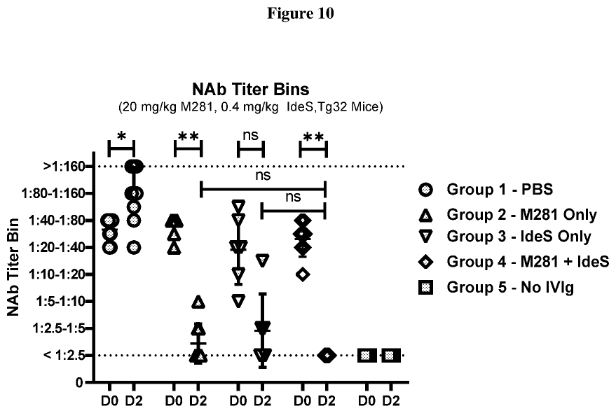

Results of the NAb assay are presented in FIG. 10 and Table 5. Several mice in the PBS group (Group 1) had increased NAb titers. The “M281 only” group (Group 2) had significantly reduced NAb titers after two days, while the “IdeS only” group (Group 3) did not show a significant reduction in NAb titers). NAb bins reflect the level at which the plasma is diluted that allows for expression of a reporter, and is a way of categorizing the NAb titers, as the values where the inhibition are in the assay can fall between certain values. The NAb titer therefore stated to be within these “bins” or levels.

Both M281 and IdeS reduced neutralizing antibody titers, with a combination therapy reducing all mice to bins below the lowest limit of detection. M281 alone was able to reduce NAbs more efficiently than IdeS.

TABLE 5
NAb Titer Bin Change

| | Median NAb | Range NAb |
|---|---|---|
| Group | Bin Change | Bin Change |
| 1 | +1 | −1 to +2 |
| 2 | −5 | −5 to −2.5 |
| 3 | −4 | −5 to +1 |
| 4 | −5 | −5 to −3 |
| 5 | 0 | 0 |

### Analysis of Anti-Spk1 Capsid IgG

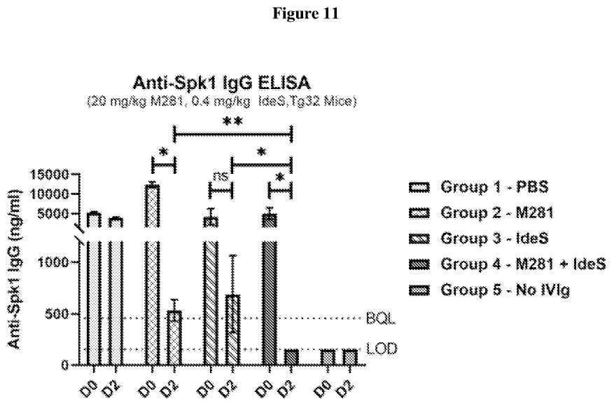

Anti-Spk1 IgG ELISA levels did not change in the “PBS only” and “no IVIg” groups between Day 0 and Day 2 (FIG. 11). Anti-Spk1 IgG levels were, on average, lowered from 5.21×10³ ng/mL (Day 0) to 3.95×10³ ng/mL (Day 2) in mice given M281 only (Group 2), and from 4.21×10³ ng/mL (Day 0) to 6.88×10² ng/mL (Day 2) in mice given IdeS only (Group 3) (Table 6). Mice that received both M281 and IdeS (Group 4) had anti-Spk1 IgG levels all drop below the limit of detection of the assay and were assigned a value of 152 ng/mL (FIG. 11). Anti-Spk1 IgG levels were significantly reduced in Group 2 (M281) and Group 4 (M281+IdeS), but not Group 3 (IdeS) from Day 0 to Day 2. Mice receiving a combination of M281 and IdeS had significantly lower anti-Spk1 IgG levels compared to M281 or IdeS alone at Day 2.

TABLE 6
Reduction of Anti-Spk1 IgG
Human Anti-Spk1 IgG ELISA

| | Day 0 | Day 2 |
|---|---|---|
| Group # | (ng/mL) ^{a} | (ng/mL) |
| 1 | 5.21 × 10³ | 3.95 × 10³ |
| 2 | 1.23 × 10⁴ | 5.31 × 10² |
| 3 | 4.21 × 10³ | 6.88 × 10² |
| 4 | 5.01 × 10³ | 1.52 × 10^{2 b} |
| 5 | 1.52 × 10^{2 b} | 1.52 × 10^{2 b} |

^{a} Mean value of all animals tested in the group
^{b} Undetectable absorbance levels by ELISA

### CONCLUSION

Thus, a combination of M281 and IdeS can reduce neutralizing antibody levels to the lowest titer bins and reduce anti-Spk1 capsid IgG levels below the limit of detection. These results provide strong support for a combination strategy of anti-FcRn agent (such as an anti-FcRn antibody) and IdeS to reduce neutralizing antibody levels in patients with high NAb titers, for improved AAV gene therapy.

These results show that an anti-FcRn monoclonal antibody (M281) and IdeS are comparable in potency for reducing anti-capsid IgG levels in a pre-clinical mouse model. Furthermore, it was demonstrated that M281 can be used with IdeS as a combination therapy to reduce anti-capsid IgG levels to undetectable levels. A combination treatment strategy of an anti-FcRn agent, such as anti-FcRn monoclonal antibody M281, and IdeS can be useful to the delivery of AAV transgenes to subjects or patients with levels of anti-capsid IgG levels that may be too high to be effectively reduced by IdeS treatment alone.

### Example 14

Mouse Study with Higher Dose of AAV Vector

The study of Example 13 is performed with a higher dose of AAV vector genomes per mouse as shown in Table 7.

TABLE 7
Groups and treatments.

| | IVIg | | Treatment | Dose | Vector | |
|---|---|---|---|---|---|---|
| Group | Dose | N | (Route) | (mg/kg) | (Route) | Dose |
| 1 | 500 | 11 | PBS (IV) | — | Spk1- | 5 × 10¹⁰ |
| 2 | mg/kg | 11 | M281 (IV) | 20 | FIX | vg/mouse |
| 3 | | 11 | IdeS (RO) | 0.4 | (IV) | |
| 4 | | 11 | M281 (IV) + | 20 + 0.4 | | |
| | | | IdeS (RO) | | | |
| 5 | PBS | 11 | PBS (IV) | — | | |

N = number of mice in group;
IV = intravenous;
RO = retroorbital;
vg = vector genomes

Additional analyses include quantificaton of vector genomes in the liver, quantification of mRNA expression of the FIX transgene in the liver, and quantification of the FIX antigen in plasma.

### Example 15

### Use of Anti-FcRn Agents to Clear NAbs and Enable AAV-Based Gene Therapy

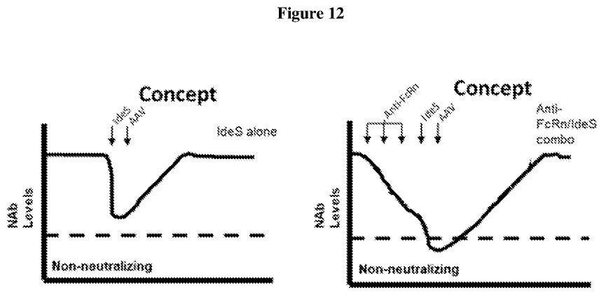

IdeS treatment alone may not eliminate a high titer of AAV NAbs, or may not eliminate or reduce the AAV NAbs enough for effective AAV transduction. In situations where a subject may have high NAbs against AAV, such as where the subject has already been dosed with a recombinant AAV vector, such high level of NAbs might not be completely eliminated by treatment with IdeS alone. Administration of an anti-FcRn agent and IdeS may more effectively clear the high titer NAbs and enable effective AAV transduction. For example, an anti-FcRn agent can be administered several times, followed by administration of IdeS, prior to administration of the AAV vector (see FIG. 12).

### Example 16

### Redosing Rabbit Study

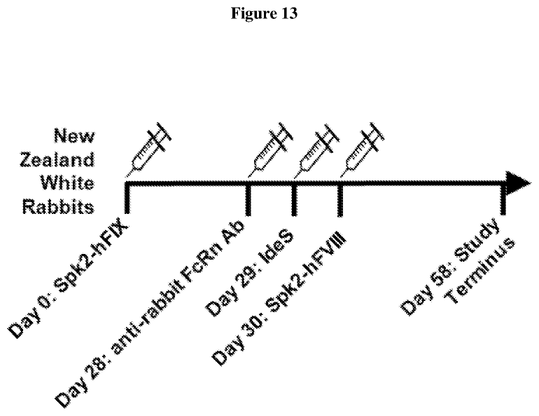

New Zealand white rabbits are initially dosed with an AAV vector carrying a transgene encoding a protein (for example, Spk2-hFIX), and are “re-dosed” with an AAV vector having the same capsid but carrying a transgene encoding a different protein (for example, Spk2-hFVIII) (FIG. 13). Alternatives include initially dosing with empty AAV vector or some other transgene, distinct from that in the “re-dosing” AAV vector. Anti-FcRn agent (such as anti-FcRn antibody M281), IdeS and combinations of an anti-FcRn agent and IdeS are administered between the first AAV dose and the second AAV dose.

TABLE 8
Groups and treatments.

| | Spk2- | | | | Spk2- |
|---|---|---|---|---|---|
| | hFIX | | | | hFVIII |
| | Dose | Number of | Treatment | Dose | Dose |
| Group | (Route) | rabbits | (Route) | (mg/kg) | (Route) |
| 1 | 1 × 10¹³ | 11 | PBS (IV) | — | 1 × 10¹³ |
| 2 | vg/rabbit | 11 | Anti-rabbit | 30 | vg/rabbit |
| | (IV) | | FcRn | | (IV) |
| | | | antibody (IV) | | |
| 3 | | 11 | IdeS (IV) | 1 | |
| 4 | | 11 | Anti-rabbit | 30 + 1 | |
| | | | FcRn | | |
| | | | antibody (IV) + | | |
| | | | IdeS (IV) | | |
| 5 | — | 11 | PBS (IV) | — | |

Measure neutralizing antibody titers, anti-capsid IgG on Day 28, 29.

Measure hFIX, hFVIII antigen and vector genomes at Day 58.

### Example 17

### Anti-FcRn Study—Cynomolgus Monkeys

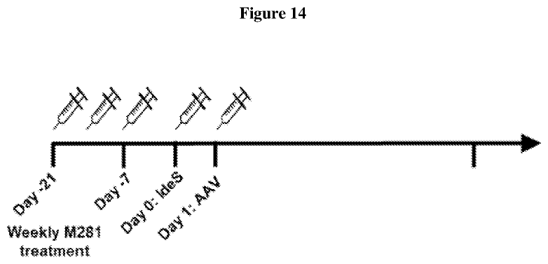

This study is designed to determine if anti-FcRn and IdeS combination treatment can enable AAV liver transduction in high titer NAb-positive cynomolgus monkeys. The schematic of the study (FIG. 14) shows weekly administration of anti-FcRn antibody (M281) for three weeks prior to administration of IdeS, and followed by AAV vector administration.

TABLE 9
Groups and treatments.

| | Number of | | | Dose | Vector |
|---|---|---|---|---|---|
| Group | Monkeys | NAbs | Treatment | (mg/kg) | (vg/kg) |
| 1 | 4 | <1:1 | PBS | — | Spk2-FVIII |
| 2 | 3 | >1:1000 | PBS | — | (2 × 10¹²) |
| 3 | 4 | | IdeS | 1 | |
| 4 | 4 | | M281 | 30 | |
| 5 | 4 | | M281/IdeS | 30/1 | |

The endpoints include bleeds between Day −21 and Day 1 to measure M281 and IdeS pharmacokinetics (PK) and pharmacodynamics (PD), multiple bleeds after AAV administration for complement analysis, weekly bleeds for clinical pathology and transgene expression, PBMCs for T-cell response, frozen tissues for biodistribution analyses; fresh tissues for immunology analyses, and fixed tissues for standard histopathology.

### Example 18

Spk1 (SEQ ID NO: 1):
MAADGYLPDWLEDNLSEGIREWWDLKPGAPKPKANQQKQDNGRGLVLPG
YKYLGPFNGLDKGEPVNAADAAALEHDKAYDQQLQAGDNPYLRYNHADA
EFQERLQEDTSFGGNLGRAVFQAKKRVLEPLGLVESPVKTAPGKKRPVE
PSPQRSPDSSTGIGKKGQQPAKKRLNFGQTGDSESVPDPQPIGEPPAAP
SGVGPNTMAAGGGAPMADNNEGADGVGSSSGNWHCDSTWLGDRVITTST
RTWALPTYNNHLYKQISNGTSGGSTNDNTYFGYSTPWGYFDFNRFHCHF
SPRDWQRLINNNWGFRPKRLNFKLFNIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQ
QNAAPIVGAVNSQGALPGMVWQNRDVYLQGPIWAKIPHTDGNFHPSPLM
GGFGLKHPPPQILIKNTPVPADPPTTFNQAKLASFITQYSTGQVSVEIE
WELQKENSKRWNPEIQYTSNYYKSTNVDFAVNTEGTYSEPRPIGTRYLT
RNL
Spk2 (SEQ ID NO: 2):
MAADGYLPDWLEDNLSEGIREWWALQPGAPKPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVD
QSPQEPDSSSGVGKSGKQPARKRLNFGQTGDSESVPDPQPLGEPPAAPT
SLGSNTMASGGGAPMADNNEGADGVGNSSGNWHCDSQWLGDRVITTSTR
TWALPTYNNHLYKQISSQSGASNDNHYFGYSTPWGYFDFNRFHCHFSPR
DWQRLINNNWGFRPKKLSFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFQFSYTFEDVPFHSSYAHSQSLDRLMNPLIDQY
LYYLNRTQGTTSGTTNQSRLLFSQAGPQSMSLQARNWLPGPCYRQQRLS
KTANDNNNSNFPWTAASKYHLNGRDSLVNPGPAMASHKDDEEKFFPMHG
NLIFGKEGTTASNAELDNVMITDEEEIRTTNPVATEQYGTVANNLQSSN
TAPTTRTVNDQGALPGMVWQDRDVYLQGPIWAKIPHTDGHFHPSPLMGG
FGLKHPPPQIMIKNTPVPANPPTTFSPAKFASFITQYSTGQVSVEIEWE
LQKENSKRWNPEIQYTSNYNKSVNVDFTVDTNGVYSEPRPIGTRYLTRP
L

Sequence of IdeS including N terminal methionine and signal sequence. (SEQ ID NO:3, NCBI Reference Sequence no. WP_010922160.1):

MRKRCYSTSAAVLAAVTLFVLSVDRGVIADSFSANQEIRYSEVTPYHVT
SVWTKGVTPPANFTQGEDVFHAPYVANQGWYDITKTFNGKDDLLCGAAT
AGNMLHWWFDQNKDQIKRYLEEHPEKQKINFNGEQMFDVKEAIDTKNHQ
LDSKLFEYFKEKAFPYLSTKHLGVFPDHVIDMFINGYRLSLTNHGPTPV
KEGSKDPRGGIFDAVFTRGDQSKLLTSRHDFKEKNLKEISDLIKKELTE
GKALGLSHTYANVRINHVINLWGADFDSNGNLKAIYVTDSDSNASIGMK
KYFVGVNSAGKVAISAKEIKEDNIGAQVLGLFTLSTGQDSWNQTN

Mature sequence of IdeS, lacking the N terminal methionine and signal sequence. (SEQ ID NO:4, Genbank accession no. ADF13949.1):

DSFSANQEIRYSEVTPYHVTSVWTKGVTPPANFTQGEDVFHAPYVANQG
WYDITKTFNGKDDLLCGAATAGNMLHWWFDQNKDQIKRYLEEHPEKQKI
NFNGEQMFDVKEAIDTKNHQLDSKLFEYFKEKAFPYLSTKHLGVFPDHV
IDMFINGYRLSLTNHGPTPVKEGSKDPRGGIFDAVFTRGDQSKLLTSRH
DFKEKNLKEISDLIKKELTEGKALGLSHTYANVRINHVINLWGADFDSN
GNLKAIYVTDSDSNASIGMKKYFVGVNSAGKVAISAKEIKEDNIGAQVL
GLFTLSTGQDSWNQTN

SEQ ID NOs:5 to 18 are the sequences of exemplary IdeS polypeptides from Table C of WO 2016/128558.

SEQ ID NO: 5:
DSFSANQEIRYSEVTPYHVTSVWTKGVTPPANFTQGEDVFHAPYVANQG
WYDITKTFNGKDDLLCGAATAGNMLHWWFDQNKDQIKRYLEEHPEKQKI
NFRGEQMFDVKEAIDTKNHQLDSKLFEYFKEKAFPYLSTKHLGVFPDHV
IDMFINGYRLSLTNHGPTPVKEGSKDPRGGIFDAVFTRGDQSKLLTSRH
DFKEKNLKEISDLIKKELTEGKALGLSHTYANVRINHVINLWGADFDSN
GNLKAIYVTDSDSNASIGMKKYFVGVNSAGKVAISAKEIKEDNIGAQVL
GLFTLSTGQDSWNQTN
SEQ ID NO: 6:
DSFSANQEIRYSEVTPYHVTSVWTKGVTPPANFTQGEDVFHAPYVANQG
WYDITKTFNGKDDLLCGAATAGNMLHWWFDQNKDQIKRYLEEHPEKQKI
NFKGEQMFDVKEAIDTKNHQLDSKLFEYFKEKAFPYLSTKHLGVFPDHV
IDMFINGYRLSLTNHGPTPVKRGSKDPRGGIFDAVFTRGNQSKLLTSRH
DFKEKNLKEISDLIKKELTEGKALGLSHTYANVRINHVINLWGADFDSN
GNLKAIYVTDSDSNASIGMKKYFVGVNSAGKVAISAKEIKEDNIGAQVL
GLFTLSTGQDSWNQTN
SEQ ID NO: 7:
DSFSANQEIRYSEVTPYHVTSVWTKGVTPPANFTQGEDVFHAPYVANQG
WYDITKTFNGKDDLLCGAATAGNMLHWWFDQNKDQIKRYLEEHPEKQKI
NFRGEQMFDVKEAIDTKNHQLDSKLFEYFKEKAFPYLSTKHLGVFPDHV
IDMFINGYRLSLTNHGPTPVKKGSKDPRGGIFDAVFTRGNQSKLLTSRH
DFKEKNLKEISDLIKKELTEGKALGLSHTYANVRINHVINLWGADFDSN
GNLKAIYVTDSDSNASIGMKKYFVGVNKAGKVAISAKEIKEDNIGAQVL
GLFTLSTGQDSWNQTN
SEQ ID NO: 8:
DSFSANQEIRYSEVTPYHVTSVWTKGVTPPANFTQGEDVFHAPYVANQG
WYDITKTFNGKDDLLCGAATAGNMLHWWFDQNKDQIKRYLREHPEKQKI
NFNGEQMFDVKEAIDTKNHQLDSKLFEYFKEKAFPYLSTKHLGVFPDHV
IDMFINGYRLSLTNHGPTPVKEGSKDPRGGIFDAVFTRGNQSKLLTSRH
DFKEKNLKEISDLIKKELDEGKALGLSHTYANVRINHVINLWGADFDSN
GNLKAIYVTDSDSNASIGMKKYFVGVNSAGKVAISAKEIKEDNIGAQVL
GLFTLSTGQDSWNQTN
SEQ ID NO: 9:
DSFSANQEIRYSEVTPYHVTSVWTKGVTPPANFTQGEDVFHAPYVANQG
WYDITKTFNGKDDLLCGAATAGNMLHWWFDQNKDQIKRYLKEHPEKQKI
NFNGEQMFDVKEAIRTKNHQLDSKLFEYFKEKAFPYLSTKHLGVFPDHV
IDMFINGYRLSLTNHGPTPVKEGSKDPRGGIFDAVFTRGNQSKLLTSRH
DFKEKNLKEISDLIKKELEEGKALGLSHTYANVRINHVINLWGADFDSN
GNLKAIYVTDSDSNASIGMKKYFVGVNKAGKVAISAKEIKEDNIGAQVL
GLFTLSTGQDSWNQTN
SEQ ID NO: 10:
DSFSANQEIRYSEVTPYHVTSVWTKGVTPPANFTQGEDVFHAPYVANQG
WYDITKTFNGKDDLLCGAATAGNMLHWWFDQNKDQIERYLEEHPEKQKI
NFNGEQMFDVKEAIDTKNHQLDSKLFEYFKEKAFPYLSTKHLGVFPDHV
IDMFINGYRLSLTNHGPTPVKEGSKDPRGGIFDAVFTRGNQSKLLTSRH
DFKEKNLKEISDLIKEELTKGKALGLSHTYANVRINHVINLWGADFDSN
GNLKAIYVTDSDSNASIGMKKYFVGVNSAGKVAISAKEIKEKNIGAQVL
GLFTLSTGQKSWNQTN
SEQ ID NO: 11:
DSFSANQEIRYSEVTPYHVTSVWTKGVTPPANFTQGEDVFHAPYVANQG
WYDITKTFNGKDDLLCGAATAGNMLHWWFDQNKDQIKRYLKEHPEKQKI
NFRGEQMFDVKEAIRTKNHQLDSKLFEYFKEKAFPYLSTKHLGVFPDHV
IDMFINGYRLSLTNHGPTPVKEGSKDPRGGIFDAVFTRGNQSKLLTSRH
DFKEKNLKEISDLIKSELENGKALGLSHTYANVRINHVINLWGADFDSN
GNLKAIYVTDSDSNASIGMKKYFVGVNKAGKVAISAKEIKEDNIGAQVL
GLFTLSTGQDSWNQTN
SEQ ID NO: 12:
DSFSANQEIRYSEVTPYHVTSVWTKGVTPPANFTQGEDVFHAPYVANQG
WYDITKTFNGKDDLLCGAATAGNMLHWWFDQNKDQIKRYLKEHPEKQKI
NFRGEQMFDVKEAIRTKNHQLDSKLFEYFKEKAFPYLSTKHLGVFPDHV
IDMFINGYRLSLTNHGPTPVKKGSKDPRGGIFDAVFTRGNQSKLLTSRH
DFKEKNLKEISDLIKKELEEGKALGLSHTYANVRINHVINLWGADFDSN
GNLKAIYVTDSDSNASIGMKKYFVGVNSAGKVAISAKEIKEDNIGAQVL
GLFTLSTGQDSWNQTN
SEQ ID NO: 13:
DSFSANQEIRYSEVTPYHVTSVWTKGVTPPANFTQGEDVFHAPYVANQG
WYDITKTFNGKDDLLCGAATAGNMLHWWFDQNKDQIERYLEEHPEKQKI
NFRGEQMFDVKEAIDTKNHQLDSKLFEYFKEKAFPYLSTKHLGVFPDHV
IDMFINGYRLSLTNHGPTPVKKGSKDPRGGIFDAVFTRGNQSKLLTSRH
DFKEKNLKEISDLIKEELTKGKALGLSHTYANVRINHVINLWGADFDSN
GNLKAIYVTDSDSNASIGMKKYFVGVNSAGKVAISAKEIKEDNIGAQVL
GLFTLSTGQKSWNQTN
SEQ ID NO: 14:
DSFSANQEIRYSEVTPYHVTSVWTKGVTPPANFTQGEDVFHAPYVANQG
WYDITKTFNGKDDLLCGAATAGNMLHWWFDQNKDQIKRYLEEHPEKQKI
NFNGEQMFDVKEAIDTKNHQLDSKLFEYFKEKAFPYLSTKHLGVFPDHV
IDMFINGYRLSLTNHGPTPVKEGSKDPRGGIFDAVFTRGDQSKLLTSRH
DFKEKNLKEISDLIKKELTEGKALGLSHTYANVRINHVINLWGADFDSN
GNLKAIYVTDSDSNASIGMKKYFVGVNSAGKVAISAKEIKEDNIGAQVL
GLFTLSTGQDSW
SEQ ID NO: 15:
SVWTKGVTPPANFTQGEDVFHAPYVANQGWYDITKTFNGKDDLLCGAAT
AGNMLHWWFDQNKDQIKRYLEEHPEKQKINFNGEQMFDVKEAIDTKNHQ
LDSKLFEYFKEKAFPYLSTKHLGVFPDHVIDMFINGYRLSLTNHGPTPV
KEGSKDPRGGIFDAVFTRGDQSKLLTSRHDFKEKNLKEISDLIKKELTE
GKALGLSHTYANVRINHVINLWGADFDSNGNLKAIYVTDSDSNASIGMK
KYFVGVNSAGKVAISAKEIKEDNIGAQVLGLFTLSTGQDSWNQTN
SEQ ID NO: 16:
SVWTKGVTPPANFTQGEDVFHAPYVANQGWYDITKTFNGKDDLLCGAAT
AGNMLHWWFDQNKDQIKRYLEEHPEKQKINFKGEQMFDVKEAIDTKNHQ
LDSKLFEYFKEKAFPYLSTKHLGVFPDHVIDMFINGYRLSLTNHGPTPV
KEGSKDPRGGIFDAVFTRGNQSKLLTSRHDFKEKNLKEISDLIKKELTE
GKALGLSHTYANVRINHVINLWGADFDSNGNLKAIYVTDSDSNASIGMK
KYFVGVNSAGKVAISAKEIKEDNIGAQVLGLFTLSTGQDSWNQTN
SEQ ID NO: 17:
SVWTKGVTPPANFTQGEDVFHAPYVANQGWYDITKTFNGKDDLLCGAAT
AGNMLHWWFDQNKDQIERYLEEHPEKQKINFKGEQMFDVKKAIDTKNHQ
LDSKLFEYFKEKAFPYLSTKHLGVFPDHVIDMFINGYRLSLTNHGPTPV
KEGSKDPRGGIFDAVFTRGNQSKLLTSRHDFKEKNLKEISDLIKEELTK
GKALGLSHTYANVRINHVINLWGADFDSNGNLKAIYVTDSDSNASIGMK
KYFVGVNSAGKVAISAKEIKEDNIGAQVLGLFTLSTGQKSWNQTN
SEQ ID NO: 18:
DDYQRNATEAYAKEVPHQITSVWTKGVTPPANFTQGEDVFHAPYVANQG
WYDITKTFNGKDDLLCGAATAGNMLHWWFDQNKDQIERYLEEHPEKQKI
NFKGEQMFDVKKAIDTKNHQLDSKLFEYFKEKAFPYLSTKHLGVFPDHV
IDMFINGYRLSLTNHGPTPVKEGSKDPRGGIFDAVFTRGNQSKLLTSRH
DFKEKNLKEISDLIKEELTKGKALGLSHTYANVRINHVINLWGADFDSN
GNLKAIYVTDSDSNASIGMKKYFVGVNSAGKVAISAKEIKEDNIGAQVL
GLFTLSTGQKSWNQTNGGGHHHHHH
IgdE of S. agalactiae specific for human IgG1
(SEQ ID NO: 19, WO2017134274):
NQNNIQETNLVEKNSEDKFIQELNRYKTEIPNFKGFNVWILGDKGYYKN
LINLEEIKNIQATLKKERNEEYVFVKLNGKIAHDTTVFLMNKKHKLLKN
IEEFKTITQKRLTERGKFPYDTVHSTFEIKDENFIMERLKSSGLSMGKP
VDYMGVNGIPIYTKTLSIDNKFAFENNSKDSSYSSNINISEDKIKENDQ
KILDLIVKSGANNQNLTDEEKVIAFTKYIGEITNYDNEAYRARNVDTEY
YRASDLFSVTERKLAMCVGYSVTAARAFNIMGIPSYVVSGKSPQGISHA
AVRAYYNRSWHIIDITASTYWKNGNYKTTYSDFIKEYCIDGYDVYDPAK
TNNRFKVKYMESNEAFENWIHNNGSKSMLFINESAALKDKKPKDDFVPV
TEKEKNELIDKYKKLLSQIPENTQNPGEKNIRDYLKNEYEEILKKDNLF
EHEHAEFKESLNLNESFYLQLKKEEKKPSDNLKKEEKPRENSVKERETP
AENNDFVSVTEKNNLIDKYKELLSKIPENTQNPGEKNIRNYLEKEYEEL
LQKDKLFKHEYTEFTKSLNLNETFYSQLKEGEMKLSENPEKGETNTN
IgdE of S. pseudoporcinus degrade both human IgG1
and porcine IgG (SEQ ID NO: 20, WO2017134274):
RENENVRQLQSENKQMKAVNLQEFSEKLKGEIAENQQFHIFKLGLNNYY
IGGVRINELSDLAKNHDFIMIDNRATHNKYGVPHIIIMNKDDVIVHNQE
DYNKEMAELTFAGDKPIQSDSYLPQKKRIHALFEIGLDSNRRQLLNAAG
LKTPENSVIELDTFKIYSHGLAVDNKYYDEYSHFNNNTNVNITKQRFTE
NDNLIHNLITTSTAKDQPTDRDKVKTFVMYVANHTIYDWNAANNAVSNI
SDVNYYLGSDLFSITERKKAMCVGFSTTAARAFNMLGIPAYVVEGKNAQ
GVDHATARVYYNGKWHTIDGTGFINGNRTRSTLYTESHFRSVGEDSYQL
VGLNEDIPFDRNYMKIDKVYEEWAPKQKTADLLLVNKDKSLVGLDRVAY
VEPVYVDKNRQDALTQIYKKLKETMESSSKKNPSSGGFSSLLGSASSDI
AKLEGSSQLTQEEYDKIHRSMTSILTFFAQLDKDAAEAFEKGNDYKNYL
ATTKHAQ

The full sequence of IdeZ available as NCBI Reference Sequence No. WP 014622780.1 (SEQ ID NO:21). This sequence includes an N-terminal methionine followed by a 33 amino acid secretion signal sequence. The N-terminal methionine and the signal sequence (a total of 34 amino acids at the N-terminus) are typically removed to form the mature IdeZ protein:

MKTIAYPNKPHSLSAGLLTAIAIFSLASSNITYADDYQRNATEAYAKEV
PHQITSVWTKGVTPLTPEQFRYNNEDVIHAPYLAHQGWYDITKAFDGKD
NLLCGAATAGNMLHWWFDQNKTEIEAYLSKHPEKQKIIFNNQELFDLKA
AIDTKDSQTNSQLFNYFRDKAFPNLSARQLGVMPDLVLDMFINGYYLNV
FKTQSTDVNRPYQDKDKRGGIFDAVFTRGDQTTLLTARHDLKNKGLNDI
STIIKQELTEGRALALSHTYANVSISHVINLWGADFNAEGNLEAIYVTD
SDANASIGMKKYFVGINAHGHVAISAKKIEGENIGAQVLGLFTLSSGKD
IWQKLS

Sequence of IdeZ without the 34 amino acids from the N-terminus of full sequence (SEQ ID NO:22):

DDYQRNATEAYAKEVPHQITSVWTKGVTPLTPEQFRYNNEDVIHAPYLA
HQGWYDITKAFDGKDNLLCGAATAGNMLHWWFDQNKTEIEAYLSKHPEK
QKIIFNNQELFDLKAAIDTKDSQTNSQLFNYFRDKAFPNLSARQLGVMP
DLVLDMFINGYYLNVFKTQSTDVNRPYQDKDKRGGIFDAVFTRGDQTTL
LTARHDLKNKGLNDISTIIKQELTEGRALALSHTYANVSISHVINLWGA
DFNAEGNLEAIYVTDSDANASIGMKKYFVGINAHGHVAISAKKIEGENI
GAQVLGLFTLSSGKDIWQKLS

The sequence of the IdeS/Z hybrid having an N-terminal part based on IdeZ, without the N-terminal methionine and the signal sequence (a total of 34 amino acids at the N-terminus) (SEQ ID NO:23):

DDYQRNATEAYAKEVPHQITSVWTKGVTPLTPEQFRYNNEDVFHAPYVA
NQGWYDITKAFDGKDNLLCGAATAGNMLHWWFDQNKDQIKRYLEEHPEK
QKINFNGDNMFDVKKAIDTKNHQLDSKLFNYFKEKAFPGLSARRIGVFP
DHVIDMFINGYRLSLTNHGPTPVKEGSKDPRGGIFDAVFTRGNQSKLLT
SRHDFKNKNLNDISTIIKQELTKGKALGLSHTYANVSINHVINLWGADF
NAEGNLEAIYVTDSDSNASIGMKKYFVGVNAHGHVAISAKKIEGENIGA
QVLGLFTLSTGQDSWQKLS

SEQ ID NOs:24-43 correspond to peptides with modifications relative to IdeZ of SEQ ID NO:22.

SEQ ID NO: 24:
DDYQRNATEAYAKEVPHQITSVWTKGVTPLTPEQFRYNNEDVIHAPYLANQGWYDITKAFDGKDNLLCGAATAGNMLHWW
FDQNKTEIEAYLSKHPEKQKIIFRNQELFDLKEAIRTKDSQTNSQLFEYFRDKAFPYLSARQLGVMPDLVLDMFINGYYL
NVFKTQSTDVKRPYQDKDKRGGIFDAVFTRGNQTTLLTARHDLKNKGLNDISTIIKEELTKGRALALSHTYANVSISHVI
NLWGADFNAEGNLEAIYVTDSDANASIGMKKYFVGINKHGHVAISAKKIEGENIGAQVLGLFTLSSGKDIWQKLN
SEQ ID NO: 25:
DDYQRNATEAYAKEVPHQITSVWTKGVTPLTPEQFRYNNEDVIHAPYLAHQGWYDITKTFNGKDNLLCGAATAGNMLHWW
FDQNKTEIEAYLSKHPEKQKIIFNNEELFDLKAAIDTKDSQTNSQLFNYFKEKAFPNLSTRQLGVMPDLVLDMFINGYYL
NVFKTQSTDVNRPYQDKDKRGGIFDAVFTRGNQTTLLTARHDFKEKGLKDISTIIKQELTEGRALALSHTYANVSISHVI
NLWGADFDAEGNLKAIYVTDSDANASIGMKKYFVGINAHGKVAISAKKIEGENIGAQVLGLFTLSSGKDIWQQLS
SEQ ID NO: 26:
DSFSANQEIRYSEVTPYHVTSVWTKGVTPLTPEQFRYNNEDVIHAPYLAHQGWYDITKAFDGKDNLLCGAATAGNMLHWW
FDQNKTEIEAYLSKHPEKQKIIFNNQELFDLKAAIDTKDSQTNSQLFNYFRDKAFPNLSARQLGVMPDLVLDMFINGYYL
NVFKTQSTDVNRPYQDKDKRGGIFDAVFTRGDQTTLLTARHDLKNKGLNDISTIIKQELTEGRALALSHTYANVSISHVI
NLWGADFNAEGNLEAIYVTDSDANASIGMKKYFVGINAHGHVAISAKKIEGENIGAQVLGLFTLSSGKDIWQKLS
SEQ ID NO: 27:
SVWTKGVTPLTPEQFRYNNEDVIHAPYLAHQGWYDITKAFDGKDNLLCGAATAGNMLHWWFDQNKTEIEAYLSKHPEKQK
IIFNNQELFDLKAAIDTKDSQTNSQLFNYFRDKAFPNLSARQLGVMPDLVLDMFINGYYLNVFKTQSTDVNRPYQDKDKR
GGIFDAVFTRGDQTTLLTARHDLKNKGLNDISTIIKQELTEGRALALSHTYANVSISHVINLWGADFNAEGNLEAIYVTD
SDANASIGMKKYFVGINAHGHVAISAKKIEGENIGAQVLGLFTLSSGKDIWQKLS
SEQ ID NO: 28:
DDYQRNATEAYAKEVPHQITSVWTKGVTPLTPEQFTQGEDVIHAPYLAHQGWYDITKAFDGKDNLLCGAATAGNMLHWWF
DQNKTEIEAYLSKHPEKQKIIFNNQELFDLKAAIDTKDSQTNSQLFNYFRDKAFPNLSARQLGVMPDLVLDMFINGYYLN
VFKTQSTDVNRPYQDKDKRGGIFDAVFTRGDQTTLLTARHDLKNKGLNDISTIIKQELTEGRALALSHTYANVSISHVIN
LWGADFNAEGNLEAIYVTDSDANASIGMKKYFVGINAHGHVAISAKKIEGENIGAQVLGLFTLSSGKDIWQKLS
SEQ ID NO: 29:
DDYQRNATEAYAKEVPHQITSVWTKGVTPPEQFTQGEDVIHAPYLAHQGWYDITKAFDGKDNLLCGAATAGNMLHWWFDQ
NKTEIEAYLSKHPEKQKIIFNNQELFDLKAAIDTKDSQTNSQLFNYFRDKAFPNLSARQLGVMPDLVLDMFINGYYLNVF
KTQSTDVNRPYQDKDKRGGIFDAVFTRGDQTTLLTARHDLKNKGLNDISTIIKQELTEGRALALSHTYANVSISHVINLW
GADFNAEGNLEAIYVTDSDANASIGMKKYFVGINAHGHVAISAKKIEGENIGAQVLGLFTLSSGKDIWQKLS
SEQ ID NO: 30:
DDYQRNATEAYAKEVPHQITSVWTKGVTPPEQFRYNNEDVIHAPYLAHQGWYDITKAFDGKDNLLCGAATAGNMLHWWFD
ANKTEIEAYLSKHPEKQKIIFNNQELFDLKAAIDTKDSQTNSQLFNYFRDKAFPNLSARQLGVMPDLVLDMFINGYYLNV
FKTQSTDVNRPYQDKDKRGGIFDAVFTRGDQTTLLTARHDLKNKGLNDISTIIKQELTEGRALALSHTYANVSISHVINL
WGADFNAEGNLEAIYVTDSDANASIGMKKYFVGINAHGHVAISAKKIEGENIGAQVLGLFTLSSGKDIWQKLS
SEQ ID NO: 31:
DDYQRNATEAYAKEVPHQITSVWTKGVTPPEQFTQGEDVIHAPYLAHQGWYDITKAFDGKDNLLCGAATAGNMLHWWFDQ
NKTEIEAYLSKHPEKQKIIINNQELFDLKAAIDTKDSQTNSQLFNYFRDKAFPNLSARQLGVMPDLVLDMFINGYYLNVF
KTQSTDVNRPYQDKDKRGGIFDAVFTRGDQTTLLTARHDLKNKGLNDISTIIKQELTEGRALALSHTYANVSISHVINLW
GADFNAEGNLEAIYVTDSDANASIGMKKYFVGINAHGHVAISAKKIEGENIGAQVLGLFTLSSGKDIWQKLS
SEQ ID NO: 32:
DDYQRNATEAYAKEVPHQITSVWTKGVTPPEQFTQGEDVIHAPYLAHQGWYDITKAFDGKDNLLCGAATAGNMLHWWFDQ
NKTEIEAYLSKHPEKQKIIFRNQELFDLKAAIDTKDSQTNSQLFNYFRDKAFPNLSARQLGVMPDLVLDMFINGYYLNVF
KTQSTDVNRPYQDKDKRGGIFDAVFTRGDQTTLLTARHDLKNKGLNDISTIIKQELTEGRALALSHTYANVSISHVINLW
GADFNAEGNLEAIYVTDSDANASIGMKKYFVGINAHGHVAISAKKIEGENIGAQVLGLFTLSSGKDIWQKLS
SEQ ID NO: 33:
DDYQRNATEAYAKEVPHQITSVWTKGVTPPEQFTQGEDVIHAPYLAHQGWYDITKAFDGKDNLLCGAATAGNMLHWWFDQ
NKTEIEAYLSKHPEKQKIIIRNQELFDLKAAIDTKDSQTNSQLFNYFRDKAFPNLSARQLGVMPDLVLDMFINGYYLNVF
KTQSTDVNRPYQDKDKRGGIFDAVFTRGDQTTLLTARHDLKNKGLNDISTIIKQELTEGRALALSHTYANVSISHVINLW
GADFNAEGNLEAIYVTDSDANASIGMKKYFVGINAHGHVAISAKKIEGENIGAQVLGLFTLSSGKDIWQKLS
SEQ ID NO: 34:
DDYQRNATEAYAKEVPHQITSVWTKGVTPPEQFTQGEDVIHAPYLANQGWYDITKAFDGKDNLLCGAATAGNMLHWWFDQ
NKTEIEAYLSKHPEKQKIIFRNQELFDLKEAIRTKDSQTNSQLFEYFRDKAFPYLSARQLGVMPDLVLDMFINGYYLNVF
KTQSTDVKRPYQDKDKRGGIFDAVFTRGNQTTLLTARHDLKNKGLNDISTIIKEELTKGRALALSHTYANVSISHVINLW
GADFNAEGNLEAIYVTDSDANASIGMKKYFVGINKHGHVAISAKKIEGENIGAQVLGLFTLSSGKDIWQKLN
SEQ ID NO: 35:
SVWTKGVTPPEQFTQGEDVIHAPYLAHQGWYDITKAFDGKDNLLCGAATAGNMLHWWFDQNKTEIEAYLSKHPEKQKIIF
RNQELFDLKAAIDTKDSQTNSQLFNYFRDKAFPNLSARQLGVMPDLVLDMFINGYYLNVFKTQSTDVNRPYQDKDKRGGI
FDAVFTRGNQTTLLTARHDLKNKGLNDISTIIKQELTEGRALALSHTYANVSISHVINLWGADFNAEGNLEAIYVTDSDA
NASIGMKKYFVGINAHGHVAISAKKIEGENIGAQVLGLFTLSSGKDIWQKLS
SEQ ID NO: 36:
DDYQRNATEAYAKEVPHQITSVWTKGVTPPEQFTQGEDVIHAPYLAHQGWYDITKAFDGADNLLCGAATAGNMLHWWFDQ
NKTEIEAYLSKHPEKQKIIFRNQELFDLKAAIDTKDSQTNSQLFNYFRDKAFPNLSARQLGVMPDLVLDMFINGYYLNVF
KTQSTDVNRPYQDKDKRGGIFDAVFTRGNQTTLLTARHDLKNKGLNDISTIIKQELTEGRALALSHTYANVSISHVINLW
GADFNAEGNLEAIYVTDSDANASIGMKKYFVGINAHGHVAISAKKIEGENIGAQVLGLFTLSSGKDIWQKLS
SEQ ID NO: 37:
SVWTKGVTPPEQFTQGEDVIHTAPYLAHQGWYDITKAFDGADNLLCGAATAGNMLHWWFDQNKTEIEAYLSKHPEKQKII
FRNQELFDLKAAIDTKDSQTNSQLFNYFRDKAFPNLSARQLGVMPDLVLDMFINGYYLNVFKTQSTDVNRPYQDKDKRGG
IFDAVFTRGNQTTLLTARHDLKNKGLNDISTIIKQELTEGRALALSHTYANVSISHVINLWGADFNAEGNLEAIYVTDSD
ANASIGMKKYFVGINAHGHVAISAKKIEGENIGAQVLGLFTLSSGKDIWQKLS
SEQ ID NO: 38:
DDYQRNATEAYAKEVPHQITSVWTKGVTPPEQFTQGEDVIHAPYLAHQGWYDITKAFDGKDNLLCGAATAGNMLHWWFDQ
NKTEIEAYLSKHPEKQKIIFRNQELFDLKAAIDTKDSQTNSQLFNYFRDKAFPNLSARQLGVMPDLVLDMFINGYYLNVF
KTQSTDVNRPYQDKDKRGGIFDAVFTRGNQTTLLTARHDLKNKGLNDISTIIKQELTEGRALALSHTYANVSISHVINLW
GADFNAEGNLEAIYVTDSDANASIGMKKYFVGINAHGHVAISAKKIEGENIGAQVLGLFTLSSGKDIWQKLS
SEQ ID NO: 39:
DDYQRNATEAYAKEVPHQITSVWTKGVTPLTPEQFTQGEDVFHAPYVANQGWYDITKAFDGKDNLLCGAATAGNMLHWWF
DQNKDQIKRYLEEHPEKQKINFNGENMFDVKKAIDTKNHQLDSKLFNYFKEKAFPYLSAKHLGVFPDHVIDMFINGYRLS
LTNHGPTPVKEGSKDPRGGIFDAVFTRGNQSKLLTSRHDFKNKNLNDISTIIKQELTKGKALGLSHTYANVRINHVINLW
GADFNAEGNLEAIYVTDSDSNASIGMKKYFVGVNAHGHVAISAKKIEGENIGAQVLGLFTLSTGQDSWQKLS
SEQ ID NO: 40:
DDYQRNATEAYAKEVPHQITSVWTKGVTPLTPEQFTQGEDVFHAPYVANQGWYDITKAFDGKDNLLCGAATAGNMLHWWF
DQNKDQIKRYLEEHPEKQKINFRGENMFDVKEAIRTKNHQLDSKLFEYFKEKAFPYLSAKHLGVFPDHVIDMFINGYRLS
LTNHGPTPVKKGSKDPRGGIFDAVFTRGNQSKLLTSRHDFKNKNLNDISTIIKSELTNGKALGLSHTYANVRINHVINLW
GADFNAEGNLEAIYVTDSDSNASIGMKKYFVGVNKHGHVAISAKKIEGENIGAQVLGLFTLSTGQDSWQKLN
SEQ ID NO: 41:
DDYQRNATEAYAKEVPHQITSVWTKGVTPLTPEQFTQGEDVFHAPYVANQGWYDITKTFNGKDDLLCGAATAGNMLHWWF
DQNKDQIKRYLEEHPEKQKINFNGEQMFDVKEAIDTKNHQLDSKLFEYFKEKAFPYLSTKHLGVFPDHVIDMFINGYRLS
LTNHGPTPVKEGSKDPRGGIFDAVFTRGNQSKLLTSRHDFKEKNLKEISDLIKQELTEGKALGLSHTYANVRINHVINLW
GADFDAEGNLKAIYVTDSDSNASIGMKKYFVGVNAAGKVAISAKKIEGENIGAQVLGLFTLSTGQDSWNQTS
SEQ ID NO: 42:
DDYQRNATEAYAKEVPHQITSVWTKGVTPLTPEQFTQGEDVFHAPYVANQGWYDITKTFNGKDDLLCGAATAGNMLHWWF
DQNKDQIKRYLEEHPEKQKINFRGEQMFDVKEAIRTKNHQLDSKLFEYFKEKAFPYLSTKHLGVFPDHVIDMFINGYRLS
LTNHGPTPVKKGSKDPRGGIFDAVFTRGNQSKLLTSRHDFKEKNLKEISDLIKEELTKGKALGLSHTYANVRINHVINLW
GADFDAEGNLKAIYVTDSDSNASIGMKKYFVGVNKAGKVAISAKKIEGENIGAQVLGLFTLSTGQDSWNQTN
SEQ ID NO: 43:
DDYQRNATEAYAKEVPHQITSVWTKGVTPPEQFTQGEDVIHAPYVANQGWYDITKAFDGKDNLLCGAATAGNMLHWWFDQ
NKDQIKRYLEEHPEKQKINFRGEQMFDVKKAIDTKNHQLDSKLFNYFKEKAFPGLSARRIGVFPDHVIDMFINGYRLSLT
NHGPTPVKEGSKDPRGGIFDAVFTRGNQSKLLTSRHDFKNKNLNDISTIIKQELTKGKALGLSHTYANVSINHVINLWGA
DFNAEGNLEAIYVTDSDSNASIGMKKYFVGVNAHGHVAISAKKIEGENIGAQVLGLFTLSTGQDSWQKLS

Protein sequence for endoglycosidase EndoS49 from Streptococcus pyogenes (SEQ ID NO:44, U.S. Pat. No. 9,493,752):

MDKHLLVKRTLGCVCAATLMGAALATHHDSLNTVKAEEKTVQTGKTDQQV
GAKLVQEIREGKRGPLYAGYFRTWHDRASTGIDGKQQHPENTMAEVPKEV
DILFVFHDHTASDSPFWSELKDSYVHKLHQQGTALVQTIGVNELNGRTGL
SKDYPDTPEGNKALAAAIVKAFVTDRGVDGLDIDIEHEFTNKRTPEEDAR
ALNVFKEIAQLIGKNGSDKSKLLIMDTTLSVENNPIFKGIAEDLDYLLRQ
YYGSQGGEAEVDTINSDWNQYQNYIDASQFMIGFSFFEESASKGNLWFDV
NEYDPNNPEKGKDIEGTRAKKYAEWQPSTGGLKAGIFSYAIDRDGVAHVP
STYKNRTSTNLQRHEVDNISHTDYTVSRKLKTLMTEDKRYDVIDQKDIPD
PALREQIIQQVGQYKGDLERYNKTLVLTGDKIQNLKGLEKLSKLQKLELR
QLSNVKEITPELLPESMKKDAELVMVGMTGLEKLNLSGLNRQTLDGIDVN
SITHLTSFDISHNSLDLSEKSEDRKLLMTLMEQVSNHQKITVKNTAFENQ
KPKGYYPQTYDTKEGHYDVDNAEHDILTDFVFGTVTKRNTFIGDEEAFAI
YKEGAVDGRQYVSKDYTYEAFRKDYKGYKVHLTASNLGETVTSKVTATTD
ETYLVDVSDGEKVVHHMKLNIGSGAIMMENLAKGAKVIGTSGDFEQAKKI
FDGEKSDRFFTWGQTNWIAFDLGEINLAKEWRLFNAETNTEIKTDSSLNV
AKGRLQILKDTTIDLEKMDIKNRKEYLSNDENWTDVAQMDDAKAIFNSKL
SNVLSRYWRFCVDGGASSYYPQYTELQILGQRLSNDVANTLKD

Protein sequence of mature Endoglycosidase S (EndoS) from S. pyogenes. (SEQ ID NO:45, Ser. No. 15/328,879, U.S. Pat. Nos. 8,889,128 and 9,707,279):

EEKTVQVQKGLPSIDSLHYLSENSKKEFKEELSKAGQESQKVKEILAKAQ
QADKQAQELAKMKIPEKIPMKPLHGPLYGGYFRTWHDKTSDPTEKDKVNS
MGELPKEVDLAFIFHDWTKDYSLFWKELATKHVPKLNKQGTRVIRTIPWR
FLAGGDNSGIAEDTSKYPNTPEGNKALAKAIVDEYVYKYNLDGLDVDVEH
DSIPKVDKKEDTAGVERSIQVFEEIGKLIGPKGVDKSRLFIMDSTYMADK
NPLIERGAPYINLLLVQVYGSQGEKGGWEPVSNRPEKTMEERWQGYSKYI
RPEQYMIGFSFYEENAQEGNLWYDINSRKDEDKANGINTDITGTRAERYA
RWQPKTGGVKGGIFSYAIDRDGVAHQPKKYAKQKEFKDATDNIFHSDYSV
SKALKTVMLKDKSYDLIDEKDFPDKALREAVMAQVGTRKGDLERFNGTLR
LDNPAIQSLEGLNKFKKLAQLDLIGLSRITKLDRSVLPANMKPGKDTLET
VLETYKKDNKEEPATIPPVSLKVSGLTGLKELDLSGFDRETLAGLDAATL
TSLEKVDISGNKLDLAPGTENRQIFDTMLSTISNHVGSNEQTVKFDKQKP
TGHYPDTYGKTSLRLPVANEKVDLQSQLLFGTVTNQGTLINSEADYKAYQ
NHKIAGRSFVDSNYHYNNFKVSYENYTVKVTDSTLGTTTDKTLATDKEET
YKVDFFSPADKTKAVHTAKVIVGDEKTMMVNLAEGATVIGGSADPVNARK
VFDGQLGSETDNISLGWDSKQSIIFKLKEDGLIKHWRFFNDSARNPETTN
KPIQEASLQIFNIKDYNLDNLLENPNKFDDEKYWITVDTYSAQGERATAF
SNTLNNITSKYWRVVFDTKGDRYSSPVVPELQILGYPLPNADTIMKTVTT
AKELSQQKDKFSQKMLDELKIKEMALETSLNSKIFDVTAINANAGVLKDC
IEKRQLLKK

Full sequence including secretion signal of endoglycosidase EndoS from Streptococcus pyogenes. (SEQ ID NO:46, AAK00850.1):

MDKHLLVKRTLGCVCAATLMGAALATHHDSLNTVKAEEKTVQVQKGLPSI
DSLHYLSENSKKEFKEELSKAGQESQKVKEILAKAQQADKQAQELAKMKI
PEKIPMKPLHGPLYGGYFRTWHDKTSDPTEKDKVNSMGELPKEVDLAFIF
HDWTKDYSLFWKELATKHVPKLNKQGTRVIRTIPWRFLAGGDNSGIAEDT
SKYPNTPEGNKALAKAIVDEYVYKYNLDGLDVDVEHDSIPKVDKKEDTAG
VERSIQVFEEIGKLIGPKGVDKSRLFIMDSTYMADKNPLIERGAPYINLL
LVQVYGSQGEKGGWEPVSNRPEKTMEERWQGYSKYIRPEQYMIGFSFYEE
NAQEGNLWYDINSRKDEDKANGINTDITGTRAERYARWQPKTGGVKGGIF
SYAIDRDGVAHQPKKYAKQKEFKDATDNIFHSDYSVSKALKTVMLKDKSY
DLIDEKDFPDKALREAVMAQVGTRKGDLERFNGTLRLDNPAIQSLEGLNK
FKKLAQLDLIGLSRITKLDRSVLPANMKPGKDTLETVLETYKKDNKEEPA
TIPPVSLKVSGLTGLKELDLSGFDRETLAGLDAATLTSLEKVDISGNKLD
LAPGTENRQIFDTMLSTISNHVGSNEQTVKFDKQKPTGHYPDTYGKTSLR
LPVANEKVDLQSQLLFGTVTNQGTLINSEADYKAYQNHKIAGRSFVDSNY
HYNNFKVSYENYTVKVTDSTLGTTTDKTLATDKEETYKVDFFSPADKTKA
VHTAKVIVGDEKTMMVNLAEGATVIGGSADPVNARKVFDGQLGSETDNIS
LGWDSKQSIIFKLKEDGLIKHWRFFNDSARNPETTNKPIQEASLQIFNIK
DYNLDNLLENPNKFDDEKYWITVDTYSAQGERATAFSNTLNNITSKYWRV
VFDTKGDRYSSPVVPELQILGYPLPNADTIMKTVTTAKELSQQKDKFSQK
MLDELKIKEMALETSLNSKIFDVTAINANAGVLKDCIEKRQLLKK

Protein sequence of EndoS isolated from S. pyogenes AP1, including signal sequence. (SEQ ID NO:47, U.S. Pat. Nos. 8,889,128 and 9,707,279):

MDKHLLVKRTLGCVCAATLMGAALATHHDSLNTVKAEEKTVQVQKGLPSI
DSLHYLSENSKKEFKEELSKAGQESQKVKEILAKAQQADKQAQELAKMKI
PEKIPMKPLHGPLYGGYFRTWHDKTSDPTEKDKVNSMGELPKEVDLAFIF
HDWTKDYSLFWKELATKHVPKLNKQGTRVIRTIPWRFLAGGDNSGIAEDT
SKYPNTPEGNKALAKAIVDEYVYKYNLDGLDVDVEHDSIPKVDKKEDTAG
VERSIQVFEEIGKLIGPKGVDKSRLFIMDSTYMADKNPLIERGAPYINLL
LVQVYGSQGEKGGWEPVSNRPEKTMEERWQGYSKYIRPEQYMIGFSFYEE
NAQEGNLWYDINSRKDEDKANGINTDITGTRAERYARWQPKTGGVKGGIF
SYAIDRDGVAHQPKKYAKQKEFKDATDNIFHSDYSVSKALKTVMLKDKSY
DLIDEKDFPDKALREAVMAQVGTRKGDLERFNGTLRLDNPAIQSLEGLNK
FKKLAQLDLIGLSRITKLDRSVLPANMKPGKDTLETVLETYKKDNKEEPA
TIPPVSLKVSGLTGLKELDLSGFDRETLAGLDAATLTSLEKVDISGNKLD
LAPGTENRQIFDTMLSTISNHVGSNEQTVKFDKQKPTGHYPDTYGKTSLR
LPVANEKVDLQSQLLFGTVTNQGTLINSEADYKAYQNHKIAGRSFVDSNY
HYNNFKVSYENYTVKVTDSTLGTTTDKTLATDKEETYKVDFFSPADKTKA
VHTAKVIVGDEKTMMVNLAEGATVIGGSADPVNARKVFDGQLGSETDNIS
LGWDSKQSIIFKLKEDGLIKHWRFFNDSARNPETTNKPIQEASLQIFNIK
DYNLDNLLENPNKFDDEKYWITVDTYSAQGERATAFSNTLNNITSKYWRV
VFDTKGDRYSSPVVPELQILGYPLPNADTIMKTVTTAKELSQQKDKFSQK
MLDELKIKEMALETSLNSKIFDVTAINANAGVLKDCIEKRQLLKK

Mature sequence of IdeS with added N-terminal methionine (SEQ ID NO:48):

MDSFSANQEIRYSEVTPYHVTSVWTKGVTPPANFTQGEDVFHAPYVANQG
WYDITKTFNGKDDLLCGAATAGNMLHWWFDQNKDQIKRYLEEHPEKQKIN
FNGEQMFDVKEAIDTKNHQLDSKLFEYFKEKAFPYLSTKHLGVFPDHVID
MFINGYRLSLTNHGPTPVKEGSKDPRGGIFDAVFTRGDQSKLLTSRHDFK
EKNLKEISDLIKKELTEGKALGLSHTYANVRINHVINLWGADFDSNGNLK
AIYVTDSDSNASIGMKKYFVGVNSAGKVAISAKEIKEDNIGAQVLGLFTL
STGQDSWNQTN

### Example 19

| SEQ ID NO for | | Description |
|---|---|---|
| anti-FcRn | | (Portion of M281 |
| antibodies | Amino Acid Sequence | antibody) |
| SEQ ID NO: 49 | Gln Ser Ala Leu Thr Gln Pro Ala Ser Val Ser Gly | VL chain |
| | Ser Pro Gly Gln Ser Ser Thr Ser Ser Cys Thr Gly | |
| | Thr Gly Ser Asp Val Gly Ser Tyr Asn Leu Val Ser | |
| | Trp Tyr Gln Gln His Pro Gly Lys Ala Pro Lys Leu | |
| | Met Ser Tyr Gly Asp Ser Glu Arg Pro Ser Gly Val | |
| | Ser Asn Arg Phe Ser Gly Ser Lys Ser Gly Asn Thr | |
| | Ala Ser Leu Thr Ser Ser Gly Leu Gln Ala Glu Asp | |
| | Glu Ala Asp Tyr Tyr Cys Ser Ser Tyr Ala Gly Ser | |
| | Gly Ser Tyr Val Phe Gly Thr Gly Thr Lys Val Thr | |
| | Val Leu Gly Gln Pro Lys Ala Ala Pro Ser Val Thr | |
| | Leu Phe Pro Pro Ser Ser Glu Glu Leu Gln Ala Asn | |
| | Lys Ala Thr Leu Val Cys Leu Ile Ser Asp Phe Tyr | |
| | Pro Gly Ala Val Thr Val Ala Trp Lys Ala Asp Ser | |
| | Ser Pro Val Lys Ala Gly Val Glu Thr Thr Thr Pro | |
| | Ser Lys Gln Ser Asn Asn Lys Tyr Ala Ala Ser Ser | |
| | Tyr Leu Ser Leu Thr Pro Glu Gln Trp Lys Ser His | |
| | Lys Ser Tyr Ser Cys Gln Val Thr His Glu Gly Ser | |
| | Thr Val Glu Lys Thr Val Ala Pro Thr Glu Cys Ser | |
| SEQ ID NO: 50 | Glu Val Gln Leu Leu Glu Ser Gly Gly Gly Leu Val | VH chain |
| | Gln Pro Gly Gly Ser Leu Arg Leu Ser Cys Ala Ala | |
| | Ser Gly Phe Thr Phe Ser Thr Tyr Ala Met Gly Trp | |
| | Val Arg Gln Ala Pro Gly Lys Gly Leu Glu Trp Val | |
| | Ser Ser Ser Gly Ala Ser Gly Ser Gln Thr Arg Tyr | |
| | Ala Asp Ser Val Lys Gly Arg Phe Thr Ser Ser Arg | |
| | Asp Asn Ser Lys Asn Thr Leu Tyr Leu Gln Met Asn | |
| | Ser Leu Arg Ala Glu Asp Thr Ala Val Tyr Tyr Cys | |
| | Ala Arg Leu Ala Ile Gly Asp Ser Tyr Trp Gly Gln | |
| | Gly Thr Met Val Thr Val Ser Ser Ala Ser Thr Lys | |
| | Gly Pro Ser Val Phe Pro Leu Ala Pro Ser Ser Lys | |
| | Ser Thr Ser Gly Gly Thr Ala Ala Leu Gly Cys Leu | |
| | Val Lys Asp Tyr Phe Pro Glu Pro Val Thr Val Ser | |
| | Trp Asn Ser Gly Ala Leu Thr Ser Gly Val His Thr | |
| | Phe Pro Ala Val Leu Gln Ser Ser Gly Leu Tyr Ser | |
| | Leu Ser Ser Val Val Thr Val Pro Ser Ser Ser Leu | |
| | Gly Thr Gln Thr Tyr Ser Cys Asn Val Asn His Lys | |
| | Pro Ser Asn Thr Lys Val Asp Lys Lys Val Glu Pro | |
| | Lys Ser Cys Asp Lys Thr His Thr Cys Pro Pro Cys | |
| | Pro Ala Pro Glu Leu Leu Gly Gly Pro Ser Val Phe | |
| | Leu Phe Pro Pro Lys Pro Lys Asp Thr Leu Met Ile | |
| | Ser Arg Thr Pro Glu Val Thr Cys Val Val Val Asp | |
| | Val Ser His Glu Asp Pro Glu Val Lys Phe Asn Trp | |
| | Tyr Val Asp Gly Val Glu Val His Asn Ala Lys Thr | |
| | Lys Pro Arg Glu Glu Gln Tyr Ala Ser Thr Tyr Arg | |
| | Val Val Ser Val Leu Thr Val Leu His Gln Asp Trp | |
| | Leu Asn Gly Lys Glu Tyr Lys Cys Lys Val Ser Asn | |
| | Lys Ala Leu Pro Ala Pro Ser Glu Lys Thr Ser Ser | |
| | Lys Ala Lys Gly Gln Pro Arg Glu Pro Gln Val Tyr | |
| | Thr Leu Pro Pro Ser Arg Glu Glu Met Thr Lys Asn | |
| | Gln Val Ser Leu Thr Cys Leu Val Lys Gly Phe Tyr | |
| | Pro Ser Asp Ser Ala Val Glu Trp Glu Ser Asn Gly | |
| | Gln Pro Glu Asn Asn Tyr Lys Thr Thr Pro Pro Val | |
| | Leu Asp Ser Asp Gly Ser Phe Phe Leu Tyr Ser Lys | |
| | Leu Thr Val Asp Lys Ser Arg Trp Gln Gln Gly Asn | |
| | Val Phe Ser Cys Ser Val Met His Glu Ala Leu His | |
| | Asn His Tyr Thr Gln Lys Ser Leu Ser Leu Ser Pro | |
| | Gly | |
| SEQ ID NO: 51 | Thr Gly Thr Gly Ser Asp Val Gly Ser Tyr Asn Leu | VL CDR1 |
| | Val Ser | |
| SEQ ID NO: 52 | Gly Asp Ser Glu Arg Pro Ser | VL CDR2 |
| SEQ ID NO: 53 | Ser Ser Tyr Ala Gly Ser Gly Ser Tyr Val | VL CDR3 |
| SEQ ID NO: 54 | Thr Tyr Ala Met Gly | VH CDR1 |
| SEQ ID NO: 55 | Ser Ser Gly Ala Ser Gly Ser Gln Thr Arg Tyr Ala | VH CDR2 |
| | Asp Ser | |
| SEQ ID NO: 56 | Leu Ala Ile Gly Asp Ser Tyr | VH CDR3 |

What is claimed is:

1. A method of enhancing the efficacy of gene therapy treatment in a subject, comprising:
(a) administering to a subject an agent that reduces the interaction of immunoglobulin G (IgG) with the neonatal Fc receptor (FcRn); and
(b) administering to said subject a recombinant viral vector comprising a therapeutic heterologous polynucleotide.

2. The method of claim 1, wherein
(a) said subject is in need of treatment for a disease caused by a loss of function or activity of a protein, and said heterologous polynucleotide encodes a polypeptide or peptide that provides or supplements a function or activity of said protein, or
(b) said subject is in need of treatment for a disease caused by a gain of function, activity or expression of a protein, and said heterologous polynucleotide is transcribed into a nucleic acid that inhibits, decreases or reduces expression of said gain of function, activity or expression of said protein.

3. The method of claim 1 or 2, wherein FcRn-mediated IgG recycling is reduced in said subject.

4. The method of any of claims 1-3, wherein IgG clearance is enhanced in said subject.

5. The method of any of claims 1-4, wherein said agent that reduces interaction of IgG with FcRn is selected from the group consisting of an anti-FcRn antibody, an FcRn binding affibody, an antibody that enhances IgG degradation (ABDEG), an FcRn binding peptide (FcBP), and an FcRn binding small molecule.

6. The method of any of claims 1-5, wherein step (a) is performed before step (b).

7. The method of any of claims 1-5, wherein step (b) is performed before step (a).

8. The method of any of claims 1-5, wherein step (a) and step (b) are performed at about the same time.

9. The method of any of claims 1-5, wherein step (a) is performed two or more times before or after step (b).

10. The method of any of claims 1-9, wherein step (b) is performed within about 90 days before or after step (a).

11. The method of any of claims 1-9, wherein step (b) is performed within about 60 days before or after step (a).

12. The method of any of claims 1-9, wherein step (b) is performed within about 45 days before or after step (a).

13. The method of any of claims 1-9, wherein step (b) is performed within about 30 days before or after step (a).

14. The method of any of claims 1-9, wherein step (b) is performed within about 21 days before or after step (a).

15. The method of any of claims 1-9, wherein step (b) is performed within about 14 days before or after step (a).

16. The method of any of claims 1-9, wherein step (b) is performed within about 7 days before or after step (a).

17. The method of any of claims 1-9, wherein step (b) is performed within about 72 hours before or after step (a).

18. The method of any of claims 1-9, wherein step (b) is performed within about 48 hours before or after step (a).

19. The method of any of claims 1-9, wherein step (b) is performed within about 24 hours before or after step (a).

20. The method of any of claims 1-9, wherein step (b) is performed within about 12 hours before or after step (a).

21. The method of any of claims 1-9, wherein step (b) is performed within about 6 hours before or after step (a).

22. The method of any of claims 1-21, further comprising administering to said subject an amount of a protease or glycosidase effective to degrade or digest and/or inhibit or reduce effector function of antibodies that bind to said recombinant viral vector and/or said polypeptide or peptide encoded by said heterologous polynucleotide and/or said heterologous polynucleotide.

23. The method of claim 22, wherein said protease or glycosidase is administered before, after or at about the same time as step (a).

24. The method of claim 22, wherein said protease or glycosidase is administered before, after or at about the same time as step (b).

25. The method of claim 22, wherein said protease or glycosidase is administered two or more times before, after or at about the same time as step (a).

26. The method of claim 22, wherein said protease or glycosidase is administered two or more times before, after or at about the same time as step (b).

27. The method of any of claims 22-26, wherein said protease or glycosidase is administered within about 90 days before or after step (a) or step (b).

28. The method of any of claims 22-26, wherein said protease or glycosidase is administered within about 60 days before or after step (a) or step (b).

29. The method of any of claims 22-26, wherein said protease or glycosidase is administered within about 45 days before or after step (a) or step (b).

30. The method of any of claims 22-26, wherein said protease or glycosidase is administered within about 30 days before or after step (a) or step (b).

31. The method of any of claims 22-26, wherein said protease or glycosidase is administered within about 21 days before or after step (a) or step (b).

32. The method of any of claims 22-26 wherein said protease or glycosidase is administered within about 14 days before or after step (a) or step (b).

33. The method of any of claims 22-26, wherein said protease or glycosidase is administered within about 7 days before or after step (a) or step (b).

34. The method of any of claims 22-26, wherein said protease or glycosidase is administered within about 72 hours before or after step (a) or step (b).

35. The method of any of claims 22-26, wherein said protease or glycosidase is administered within about 48 hours before or after step (a) or step (b).

36. The method of any of claims 22-26, wherein said protease or glycosidase is administered within about 24 hours before or after step (a) or step (b).

37. The method of any of claims 22-26, wherein said protease or glycosidase is administered within about 12 hours before or after step (a) or step (b).

38. The method of any of claims 22-26, wherein said protease or glycosidase is administered within about 6 hours before or after step (a) or step (b).

39. The method of any of claims 22-38, wherein said protease comprises a cysteine protease or a thiol protease.

40. The method of any of claims 22-38, wherein said protease comprises a protease from Streptococcus pyogenes, Streptococcus equi or Mycoplasma canis.

41. The method of any of claims 22-38, wherein said protease comprises IdeS or a modified variant thereof set forth in any of SEQ ID NOs:3-18, 23 or 48.

42. The method of any of claims 22-38, wherein said glycosidase comprises an endoglycosidase.

43. The method of claim 42, wherein said endoglycosidase comprises a sequence set forth in any of SEQ ID NOs: 44-47.

44. The method of any of claims 22-43, wherein said protease or glycosidase degrades or digests and/or inhibits or reduces effector function of human antibodies.

45. The method of any of claims 1-44, wherein said viral vector comprises a lentiviral vector, an adenoviral vector or an adeno-associated virus (AAV) vector.

46. The method of claim 45, wherein said lentiviral vector comprises envelope proteins to which said antibodies or IgG bind.

47. The method of claim 45, wherein said AAV vector comprises capsid proteins to which the antibodies or IgG bind.

48. The method of claim 45, wherein said AAV vector comprises VP1, VP2 and/or VP3 capsid proteins to which the antibodies or IgG bind.

49. The method of any of claims 45-48, wherein said AAV vector comprises VP1, VP2 and/or VP3 capsid protein having 60% or more sequence identity to VP1, VP2 and/or VP3 capsid protein selected from the group consisting of AAV1, AAV2, AAV3, AAV4, AAV5, AAV6, AAV7, AAV8, AAV9, AAV10, AAV3B, AAV-2i8, Rh10, Rh74, SEQ ID NO:1 and SEQ ID NO:2 VP1, VP2 and/or VP3 capsid proteins.

50. The method of any of claims 45-48, wherein said AAV vector comprises VP1, VP2 and/or VP3 capsid protein having 100% sequence identity to VP1, VP2 and/or VP3 capsid protein selected from the group consisting of AAV1, AAV2, AAV3, AAV4, AAV5, AAV6, AAV7, AAV8, AAV9, AAV10, AAV3B, AAV-2i8, Rh10, Rh74, SEQ ID NO:1 and SEQ ID NO:2 VP1, VP2 and/or VP3 capsid proteins.

51. The method of any of claims 1-50, wherein said subject has antibodies or IgG that bind to said viral vector.

52. The method of any of claims 1-50, wherein antibodies or IgG that bind to said viral vector are absent from said subject.

53. The method of any of claims 1-52, wherein said subject has antibodies or IgG that bind to said polypeptide or peptide encoded by said heterologous polynucleotide.

54. The method of any of claims 22-53, wherein said antibodies comprise IgG, IgM, IgA, IgD and/or IgE.

55. The method of any of claims 1-54, further comprising determining the presence of, quantifying the amount of or an effector function of viral vector binding antibodies or IgG present in said subject before performing step (a), after performing step (a) but before performing step (b) and/or after performing steps (a) and (b).

56. The method of any of claims 1-54, further comprising analyzing a biological sample from said subject for the presence, amount or an effector function of viral vector binding antibodies or IgG present in said sample before performing step (a), after performing step (a) but before performing step (b) and/or after performing steps (a) and (b).

57. The method of claim 55 or 56, wherein said determining and/or analyzing step is carried out before and/or after administration of said protease or glycosidase.

58. The method of claim 56 or 57, wherein said biological sample from said subject is a blood product.

59. The method of any of claims 1-58, wherein said method leads to a reduction of 20-50%, 50-75%, 75-90%, 90-95% or 95% or more of said viral vector binding antibodies or IgG.

60. The method of any of claims 56-58, wherein said viral vector binding antibodies or IgG present in said biological sample or blood product from said subject is less than about 1:100,000 where 1 part of said biological sample or blood product diluted in 100,000 parts of buffer results in 50% viral vector neutralization.

61. The method of any of claims 56-58, wherein said viral vector binding antibodies or IgG present in said biological sample or blood product from said subject is less than about 1:50,000, where 1 part of said biological sample or blood product diluted in 50,000 parts of buffer results in 50% viral vector neutralization.

62. The method of any of claims 56-58, wherein said viral vector binding antibodies or IgG present in said biological sample or blood product from said subject is less than about 1:10,000, where 1 part of said biological sample or blood product diluted in 10,000 parts of buffer results in 50% viral vector neutralization.

63. The method of any of claims 56-58, wherein said viral vector binding antibodies or IgG present in said biological sample or blood product from said subject is less than about 1:1,000, where 1 part of said biological sample or blood product diluted in 1,000 parts of buffer results in 50% viral vector neutralization.

64. The method of any of claims 56-58, wherein said viral vector binding antibodies or IgG present in said biological sample or blood product from said subject is less than about 1:100, where 1 part of said biological sample or blood product diluted in 100 parts of buffer results in 50% viral vector neutralization.

65. The method of any of claims 56-58, wherein said viral vector binding antibodies or IgG present in said biological sample or blood product from said subject is less than about 1:10, where 1 part of said biological sample or blood product diluted in 10 parts of buffer results in 50% viral vector neutralization.

66. The method of any of claims 56-58, wherein said viral vector binding antibodies or IgG present in said biological sample or blood product is less than about 1:5, where 1 part of said biological sample or blood product diluted in 5 parts of buffer results in 50% viral vector neutralization.

67. The method of any of claims 56-58, wherein the ratio of viral vector binding antibodies or IgG present in said biological sample or blood product is less than about 1:4, where 1 part of said biological sample or blood product diluted in 4 parts of buffer results in 50% viral vector neutralization.

68. The method of any of claims 56-58, wherein the ratio of viral vector binding antibodies or IgG present in said biological sample or blood product is less than about 1:3, where 1 part of said biological sample or blood product diluted in 3 parts of buffer results in 50% viral vector neutralization.

69. The method of any of claims 1-58, wherein the ratio of viral vector binding antibodies or IgG present in said subject, biological sample or blood product is less than about 1:2, where 1 part of said biological sample or blood product diluted in 2 parts of buffer results in 50% viral vector neutralization.

70. The method of any of claims 1-58, wherein the ratio of viral vector binding antibodies or IgG present in said subject, biological sample or blood product is less than about 1:1, where 1 part of said biological sample or blood product diluted in 1 part of buffer results in 50% viral vector neutralization.

71. The method of any of claims 1-70, further comprising determining the presence of or quantifying the amount of antibodies or IgG that bind to a polypeptide or peptide encoded by said heterologous polynucleotide, after performing step (a) but before performing step (b) and/or after performing steps (a) and (b).

72. The method of any of claims 1-70, further comprising determining the presence of or quantifying the amount of antibodies or IgG that bind to said heterologous polynucleotide or nucleic acid after performing step (a) but before performing step (b) and/or after performing steps (a) and (b).

73. The method of any of claims 1-72, wherein said subject has a lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, a lysosomal storage disease, (e.g., aspartylglucosaminuria, Batten disease, late infantile neuronal ceroid lipofuscinosis type 2 (CLN2), cystinosis, Fabry disease, Gaucher disease types I, II, and III, glycogen storage disease II (Pompe disease), GM2-gangliosidosis type I (Tay Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A, B, C, D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), a disease of solid organs (e.g., brain, liver, kidney, heart), or an infectious viral (e.g., hepatitis B and C, HIV, etc.), bacterial or fungal disease.

74. The method of any of claims 1-72, wherein said subject has a blood clotting disorder.

75. The method of any of claims 1-72, wherein said subject has hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

76. The method of any of claims 1-72, wherein said subject has anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors), or a platelet disorder such as, Bernard Soulier syndrome, Glanzmann thrombasthenia, or storage pool deficiency.

77. The method of any of claims 1-72, wherein said heterologous polynucleotide encodes a protein selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

78. The method of any of claims 1-72, wherein said heterologous polynucleotide encodes a protein selected from the group consisting of thrombopoietin (TPO), interleukins (IL1 through IL-36), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

79. The method of any of claims 1-72, wherein said heterologous polynucleotide encodes CFTR (cystic fibrosis transmembrane regulator protein), a blood coagulation (clotting) factor (Factor XIII, Factor IX, Factor VIII, Factor X, Factor VII, Factor VIIa, protein C, etc.) a gain of function blood coagulation factor, an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, 0-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, a sulfatase, N-acetylglucosamine-1-phosphate transferase, cathepsin A, GM2-AP, NPC1, VPC2, a sphingolipid activator protein, one or more zinc finger nuclease for genome editing, and one or more donor sequence used as repair templates for genome editing.

80. The method of any of claims 1-72, wherein said heterologous polynucleotide encodes an inhibitory nucleic acid.

81. The method of claim 80, wherein said inhibitory nucleic acid is selected from the group consisting of a siRNA, an antisense molecule, miRNA, RNAi, a ribozyme and a shRNA.

82. The method of claim 80, wherein said inhibitory nucleic acid binds to a gene, a transcript of a gene, or a transcript of a gene associated with a polynucleotide repeat disease selected from the group consisting of a huntingtin (HTT) gene, a gene associated with dentatorubropallidoluysian atrophy (atrophin 1, ATN1), androgen receptor on the X chromosome in spinobulbar muscular atrophy, human Ataxin-1, -2, -3, and -7, Caᵥ2.1 P/Q voltage-dependent calcium channel (CACNA1A), TATA-binding protein, Ataxin 8 opposite strand (ATXN8OS), Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DNA damage-inducible transcript 4 protein, in diabetic macular edema (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; and mutant rhodopsin gene (RHO) in autosomal dominantly inherited retinitis pigmentosa (adRP).

83. The method of any of claims 1-82, wherein said polypeptide encoded by said heterologous polynucleotide comprises a gene editing nuclease.

84. The method of claim 83, wherein said gene editing nuclease comprises a zinc finger nuclease (ZFN) or a transcription activator-like effector nuclease (TALEN).

85. The method of claim 83, wherein said gene editing nuclease comprises a functional Type II CRISPR-Cas9.

86. The method of any of claims 1-85, wherein step (a) and/or step (b) are performed two or more times.

87. The method of any of claims 1-86, wherein said subject is a human.

88. A package having disposed therein:
(a) a recombinant viral vector comprising a heterologous polynucleotide that encodes a polypeptide or peptide;
(b) an agent that reduces interaction of IgG with FcRn;
(c) optionally, a protease or glycosidase that degrades or digests antibodies; and
(d) a label with instructions for performing a method according to any of claims 1-87, wherein (a), (b) and (c) are in separate or the same container.

89. A package having disposed therein:
(a) a recombinant viral vector comprising a heterologous polynucleotide that is transcribed into a nucleic acid that inhibits, decreases or reduces expression of a protein;
(b) an agent that reduces interaction of IgG with FcRn;
(c) optionally, a protease or glycosidase that degrades or digests antibodies; and
(d) a label with instructions for performing a method according to any of claims 1-87, wherein (a), (b) and (c) are in separate or the same container.
